(19) United States
(12) Patent Application Publication
(10) Pub. No.: US 2010/0137412 A1
(43) Pub. Date: Jun. 3, 2010

(54) LENTIVIRUS PSEUDOTYPED WITH INFLUENZA HEMAGGLUTININ AND METHODS OF USE

(75) Inventors: Paul ZHOU, Shanghai (CN); Cheguo Tsai, Shanghai (CN); Tetsuya Toyoda, Shanghai (CN); Philippe Buchy, Juan-les-pins (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: INSTITUT PASTEUR OF SHANGHAI, Shanghai (CN)

(21) Appl. No.: 12/493,638

(22) Filed: Jun. 29, 2009

Related U.S. Application Data
(63) Continuation-in-part of application No. PCT/IB2008/001097, filed on Jan. 2, 2008.

(51) Int. Cl. A61K 31/7088; C12N 15/74; C12N 5/10; C12Q 1/70; A61P 31/12; A61P 37/04

(52) U.S. Cl. 514/44 R; 435/320.1; 435/358; 435/5

(57) ABSTRACT

Highly effective pseudotyping of lentiviral vector with influenza HA, NA and M2 packaging gene constructs at the proper ratios. Lentivirus vector pseudotyped with influenza HA, especially pseudotyped with H5 and neuraminidase. Methods of inducing immune responses to influenza antigens or for transducing genes into cells to which influenza antigens bind using such lentivirus vectors. Methods for screening drugs which inhibit influenza infection using lentivirus pseudotyped with HA.

The current invention relates to lentiviruses pseudotyped with viral proteins from other types of viruses, such as influenza virus hemagglutinin (HA), neuraminidase (NA) or M2 protein. The invention also relates to modified lentivirus vectors and gene delivery systems; and antigens, immunogens or vaccines using pseudotyped lentiviruses; and methods for inducing immunity or detecting viral products using pseudotyped lentivirus; and pseudotyped lentivirus-based methods for screening molecules for antiviral activity or for an ability to block viral entry into host cells.

A virus is pseudotyped when an envelope protein normally expressed by the virus is replaced with an exogenous envelope protein from a different virus or with a chimeric or hybrid envelope protein. Pseudotyping confers new properties on a virus, such as changing its ability to bind to host cells, modifying its natural host range or allowing it to transfer additional genetic information into host cells.

Lentiviruses represent one genus in the family of Retroviruses. Their basic structure includes an RNA genome contained within a core on or through which receptor-binding envelope proteins are arranged. An engineered lentivirus vector exhibits some or all of the characteristics of a lentivirus, but can include alterations in the lentivirus structure modifying the functional characteristics of the native lentivirus from which it is derived. For example, the RNA genome of the lentivirus vector may be modified to include exogenous polynucleotide sequences or transgenes for incorporation into a target host cell. The envelope proteins of a native lentivirus may be pseudotyped by the replacement with the envelope proteins of an exogenous virus thus modifying the host range of the lentivirus vector.

Lentivirus vectors may be replication competent or replication incompetent. A replication competent vector encodes all the materials it needs to infect a host cell and reproduce itself, while a replication incompetent vector cannot. A replication incompetent vector may be preferred for biological safety and for its generally higher capacity to carry more exogenous genetic material than a replication competent vector.

Methods for making lentivirus vectors, pseudotyping lentivirus envelope proteins and using such vectors for transducing polynucleotide sequences are known in the art and are also incorporated by reference to the following publications.

Kingsman, et al., U.S. Pat. No. 6,669,936 describes infection and transduction competent lentivirus vectors which lack functional lentivirus auxiliary gene products.

Leboulch, et al., U.S. Pat. No. 6,365,150 describes lentivirus packaging cells which produce recombinant lentivirus providing increased safety by virtually eliminating the possibility of molecular recombination leading to the production of replication-competent helper virus.

Marasco, et al., U.S. Pat. No. 6,830,892, describes lentivirus vectors useful for screening target molecules.

Marasco, et al., U.S. Pat. No. 7,078,031 describes pseudotyped lentiviral vectors and gene delivery using these vectors.

Spencer, et al., U.S. Pat. No. 7,090,837 describes lentivirus packaging constructs and packaging systems and gene transduction using lentivirus vectors.

McKay et al., Gene Ther. 13:715 (2006) describes lentivirus-based gene transfer using influenza hemagglutinin (HA) from fowl plague virus (FPV, H7/Rostok).

Matrosovich et al. (25) indicates that NA plays an important role in early phase of virus infection.

Such vectors and methods of their use are also incorporated by reference to Current Protocols in Molecular Biology, volume 1 (Nov. 20, 2006), see especially Chapter 9 “Introduction of DNA into Mammalian Cells” or by reference to the documents cited above or in the reference section below.

Lentivirus vectors may also include one or more reporter genes, such as a polynucleotide encoding green fluorescent protein (GFP). Suitable reporter genes, methods for incorporating reporter genes into lentivirus vectors and methods for detecting reporter gene activity are well known in the art and are incorporated by reference to Current Protocols in Molecular Biology, volume 1 (Nov. 20, 2006), see especially Chapter 9, Part II, “Uses of fusion genes in mammalian transfection”.

Lentiviral vectors pseudotyped with envelope proteins from other viruses provide a powerful tool for a variety of basic science and clinical applications. First, as a gene delivery system it can direct gene transfer into desirable tissues and cells in vitro and in vivo (1, 2). Second, as a tool for basic research it can be used to uncover the molecular mechanism of envelope protein mediated cell entry (3). Third, as an immunogen, it can be used in vaccine development against infectious diseases and cancers (4, 5). Fourth, as an antigen, it has been used to develop a novel neutralizing assay to measure antibody response during the course of infection and vaccination (6, 7). Finally, as a vehicle of cell entry, it can be used to develop high-throughput systems to screen entry blockers, thereby help in the development of new anti-viral drugs (3).

The G glycoprotein from vesicular stomatitis virus (VSV-G) is widely used for pseudotyping lentiviral viruses due to its high efficiency in pseudotyping lentiviral vectors to target gene transfer to a broad range of cells and tissues (8-10). However, some cells and tissues such as the apical membrane of polarized epithelia or mucosal tissue are refractory to lentiviral vectors pseudotyped with VSV-G (1, 11).

To overcome this limitation, efforts have been made to pseudotype viruses with envelope proteins from other viruses such as filoviruses (1, 2), orthomyxoviruses (11, 12), paramyxoviruses (13), hepatitis C virus (14), and other retroviruses (12, 15).

Influenza viruses are members of the orthomyxovirus family of RNA viruses. Influenza, commonly known as flu, is an infectious disease of birds and mammals. In humans, common symptoms of influenza are fever, sore throat, muscle pains, severe headache, coughing, weakness and general discomfort. In more serious cases, influenza causes pneumonia, which can be fatal, particularly in young children and the elderly.

There are three types of influenza, designated influenza A, B and C (two other members of this family, the Dhori and Thorgoto viruses are borne by ticks and are rarely encountered). Influenza A viruses (which include the avian or bird viruses) cause the most severe disease in humans, although influenza B also regularly causes outbreaks.

The A, B and C designations originally referred to broad classes of antibody response to the virus and are now known also to be related to genetic differences in the respective M1 (capsid or matrix protein) or the nucleoprotein (NP) of the three virus types. Studies of the genetic sequences of these viruses indicate that at some time they all had a common ancestor. The H5N1 bird flu virus belongs to the influenza A class or type.

The type (A, B or C) is the first important part of the influenza virus name. Then comes the sub-type, which is named for the broad classes of the hemagglutinin (HA) or neuraminidase (NA) surface proteins projecting through the viral envelope. There are 16 HA sub-types (designated H1-H16) and 9 NA sub-types (designated N1-N9). All of the possible combinations of these influenza A subtypes infect birds, but only those containing the H1, H2, H3, H5, H7 and H9 and the N1, N2 and N7 surface proteins infect humans and of these, so far, only H1, H2, H3 and N1 and N2 do so to any extent. The H5 subtype is considered a candidate for a new subtype for broad human infectivity. Since this subtype is “new” to the immune systems of most people on the globe, if this subtype becomes broadly infective for humans, it is likely to result in a pandemic, that is to produce a wave of infection around the world.

Full naming of an influenza A virus thus includes both the type and the subtype e.g., influenza A/H5N1 or influenza A/H3N2; these may also be written using parentheses instead of slashes, i.e. A(H3N2) etc. In the current application as all influenza virus types discussed are type A, viruses are simply designated using the subtype combination i.e. H3N2.

Three important Influenza proteins are hemagglutination (HA), neuraminidase (NA) and M2 which are envelope proteins on the surface of the influenza virus.

On the surface of a mature influenza virion, the HA spike is a trimeric complex of HA₁ and HA₂ heterodimers (16, 17). It binds to sialic acid-containing receptors on the target cell surface and is responsible for penetration of the virus into the cell cytoplasm by mediating the fusion of the membrane of endocytosed virus with the endosomal membrane (18, 19).

HA is initially synthesized on membrane-bound ribosomes and translocated into the lumen of the endoplasmic reticulum as a single polypeptide precursor HA₀ and then cleaved into two disulfide-linked chains HA₁ and HA₂.

One form of HA₀ possesses multiple basic amino acids at the carboxyl terminus of HA₁, it is cleaved by a cellular endopeptidase located in the trans-Golgi network (TGN) (20, 21). A second form of HA₀ does not possess multiple basic amino acids at the carboxyl terminus of HA₁, it is cleaved in vivo by one of two groups of proteases: plasmin, a blood-clotting factor X-like protease, and tryptase Clara, a product of specialized respiratory epithelial cells (22-24).

On the surface of the mature influenza virion NA is present as a homo-tetramer. It catalyzes the cleavage of the α-ketosidic linkage between a terminal sialic acid and an adjacent D-galactose or D-galactosamine (25). One function of NA is to remove sialic acid from HA, NA, and the cell surface (26). It may also permit transport of the virus through the mucin layer present in the respiratory tract so that the virus can target epithelial cells (27). Some avian influenza NA proteins also have a receptor binding site that causes hemagglutination, although the role of this receptor binding function in the life cycle of influenza virus is still unknown (28). Recently, it was found that NA also plays an important role in early phase of virus infection (29).

About 20 to 60 M2 protein molecules are expressed as homotetramers on the surface of the mature virion. These function as ion channels that permits ions to enter the virion during uncoating and also act as an ion channel which modulates the pH of TGN and transported vesicles (30). Interestingly, so far an avian flu strain A/chicken/Germany/34 (H7N1) fowl plague virus (FPV) Rostock is the only strain whose HA depends on the ion channel activity of M2 in TGN and transported vesicles to maintain the right conformation during its biogenesis (31, 32). Without M2, FPV H7HA is expressed on the ER and seldom reaches to the cell surface (11, 32).

FPV H7HA has been used to pseudotype retrovirus and EIAV- or HIV-1-based lentiviral vectors (11, 33). Recently, McKay et al. (11) reported that M2 significantly augments FPV H7HA pseudotyping of lentiviral vector. In addition, they showed that treatment of cells producing FPV H7HA/M2 pseudotyped lentivirus with soluble bacterial NA or co-expression of cDNA encoding NA enhances the pseudovirion release from producer cells. Finally, they demonstrated that this FPV H7HA/M2 pseudotyped lentivirus efficiently transduces the apical membrane of polarized mouse tracheal culture ex vivo as well as mouse tracheal epithelia in vivo.

As indicated above 16 HA subtypes have been identified in avians. Among them HA from serotypes 1, 2, and 3 has been transmitted into humans and spread from human to human; whereas HA from serotypes 5, 7, and 9 has also been transmitted into human, but human-to-human spreading has not been reported so far (34) although in human airways both sialyloligosaccharides terminated by SAα2,6 galactose and by SAα2,3 galactose were found (35).

As mentioned above, FPV H7HA, which binds SAα2,3 galactose, has a unique feature in its dependency on M2 during biogenesis (31, 32). Prior to the present invention, whether M2 and NA were required for lentiviral vectors pseudotyped with HA from other viral strains was not known.

The inventors therefore have set out to improve pseudotyped lentiviruses and vectors derived from these, by investigating the effects of various HA sub-types when introduced into a lentivirus as well as the effects of NA and M2.

In particular the current invention relates to a lentivirus vector pseudotyped with:

an influenza HA protein or a protein containing an HA protein fragment comprising an HA epitope or an HA cellular attachment ligand,

wherein said HA protein is not fowl plague virus H7.

As described herein such a pseudotyped lentivirus vector has many beneficial properties, in particular the inventors have discovered that a lentivirus vector pseudotyped with HA alone allows transduction of a desired polynucleotide sequence or transgene into a target cell to which HA binds.

Preferably the HA protein is selected from the group consisting of H1, H2, H5 and H7 as defined above (i.e. not fowl plague virus H7) and more preferably from the group consisting of H1, H2 and H5. In particular:
the H1 protein may comprise the peptide sequence of SEQ ID NO: 3 and SEQ ID NO: 35; or a corresponding nucleic acid coding sequence such as SEQ ID NO: 16 and SEQ ID NO: 45;
the H2 protein may comprise the peptide sequence of SEQ ID NO: 1; a corresponding nucleic acid coding sequence is SEQ ID NO: 18; or
the H5 protein may comprise the peptide sequence of one of SEQ ID NO: 4, SEQ ID NO: 5, SEQ ID NO: 6 SEQ ID NO: 7, SEQ ID NO: 27, SEQ ID NO: 28, SEQ ID NO: 29 SEQ ID NO: 30, SEQ ID NO: 31, SEQ ID NO: 32, SEQ ID NO: 33 SEQ ID NO: 34; or a corresponding nucleotide coding sequence such as are SEQ ID NO: 17, SEQ ID NO: 19, SEQ ID NO: 20, SEQ ID NO: 21, SEQ ID NO: 37, SEQ ID NO: 38, SEQ ID NO: 39 SEQ ID NO: 40, SEQ ID NO: 41, SEQ ID NO: 42, SEQ ID NO: 43 SEQ ID NO: 44.

Other slightly different nucleic acids, when able to express the hereabove proteins, may also be used and can be deduced from the said proteins by known methods.

The HA protein is one determinant of influenza virulence and target specificity and has hence been subject to extensive and prolonged investigation. The various forms of HA isolated to date are each important and so the new pseudotyped lentiviral materials with such HA molecules of the current invention provide various advantages as detailed in the current application.

Preferably the lentivirus further comprises NA. In particular the NA protein may comprise the peptide sequence of SEQ ID NO: 8, SEQ ID NO: 9, SEQ ID NO: 10, SEQ ID NO: 11, SEQ ID NO: 26; corresponding nucleotide coding sequence are SEQ ID NO: 22, SEQ ID NO: 23, SEQ ID NO: 24, SEQ ID NO: 25; SEQ ID NO: 36.

While the pseudotyped lentivirus needs only HA to bind to the target cell and transduce its genetic material, the inventors have discovered that substantial increases in transduction efficiency result when NA is incorporated in the pseudotyped virus in addition to HA.

Preferably the lentivirus vector comprises NA from an H5N1 avian flu strain.

Preferably the lentivirus vector further comprising NA and M2. Further increases in transduction efficiency may be achieved by including both NA and M2 in the pseudotyped virus along with HA. In particular the M2 protein is encoded by the nucleotide coding sequence of SEQ ID NO: 2.

Co-transfecting NA alone, but not M2 alone, with H5HA resulted in an increase of cell surface expression of H5HA and dramatic (4 to 5 logs) increase in transduction efficiency. The best transduction efficiency was obtained when the ratio of HA and NA constructs ranged between 4:1 and 8:1. In addition, cotransfection of M2 with H5HA and NA provided an additional moderate (2-to-3-fold) increase in transduction efficiency.

The inventors have discovered that NA, but not M2, dramatically enhances transduction efficiency of H5HA pseudotypes. The H5HA/NA/M2 pseudotypes mimic the early infection step of influenza virus, which makes them suitable for developing a high through-put assay to evaluate neutralizing antibody response to as well as to screen entry blockers of avian flu viruses.

Like wild type avian flu viruses which expressing H5, the H5HA/NA/M2 lentivirus pseudotypes entered cells through receptor-mediated endocytosis and cell entry is effectively neutralized by immune sera specific for H5HA. Specifically the entry by H5HA/NA/M2 pseudotypes can be neutralized by immune sera in mice specific for H5HA as well as by convalescent sera from H5N1-infected, but recovered human patients.

H5HA/NA/M2 pseudotypes transduce genetic material into a broad range of cells with efficiency compatible to VSV-G pseudotypes making such pseudotyped lentiviral vectors of great potential value as a new type of transfection reagent.

At least in two aspects these results are quite different from those recently reported by McKay et al. on PFV H7HA pseudotyping of lentiviral vectors (11). First, in their report M2 has been shown to be required for the cell surface expression of PFV H7HA. Without co-transfection of M2, PFV H7HA could only be detected intracellularly, which indicates the impairment of trafficking of PFV H7HA through the secretory pathway.

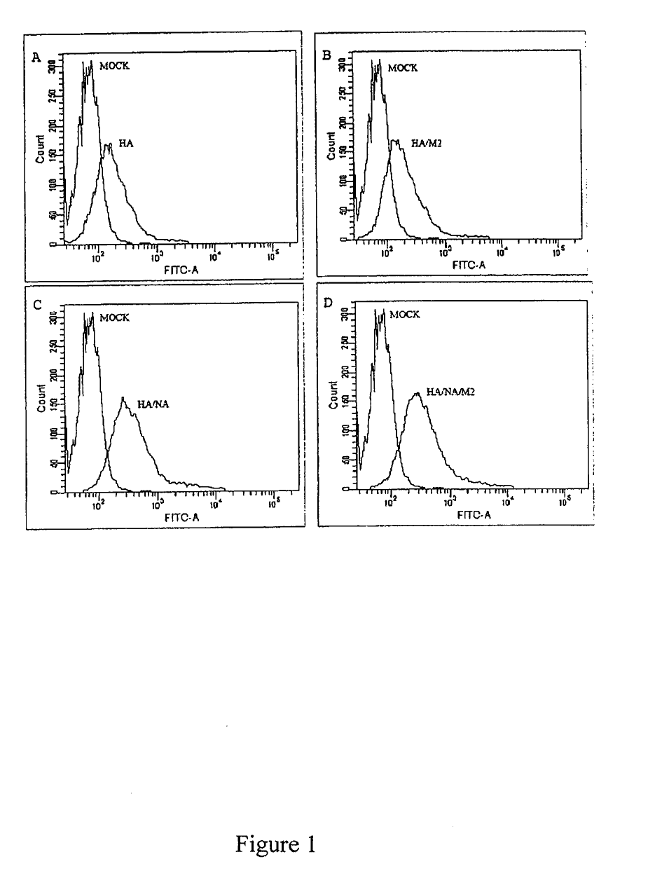

In contrast, the inventors have discovered that expression of M2 is not necessary to obtain the surface expression of other HA proteins like H5 on the surface of packaging cells (FIG. 1A).

While not being bound to any particular explanation, the difference in dependency of M2 on the surface expression of packaging cells between H5HA and PFV H7HA is consistent with what was previously known about the biogenesis of HA (27). So far PFV H7HA is the only HA whose biogenesis depends on the ion channel activity of M2 in TGN and transported vesicles to maintain the right conformation (27).

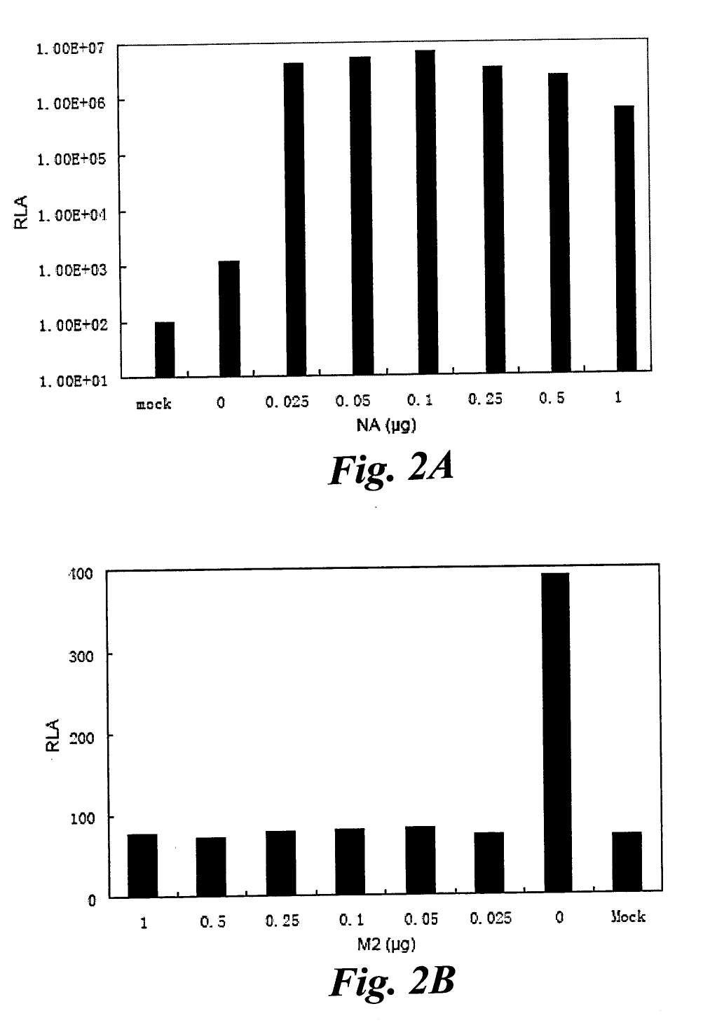

Second, McKay et al. indicate that M2 and NA synergize (about 750 folds) efficient transduction of PFV H7HA pseudotyped lentiviral vectors. However, in the present invention, it was found that co-transfection of NA, but not M2, 4 to 5 log increases the transduction efficiency of H5HA pseudotyped lentiviral vectors (FIG. 2).

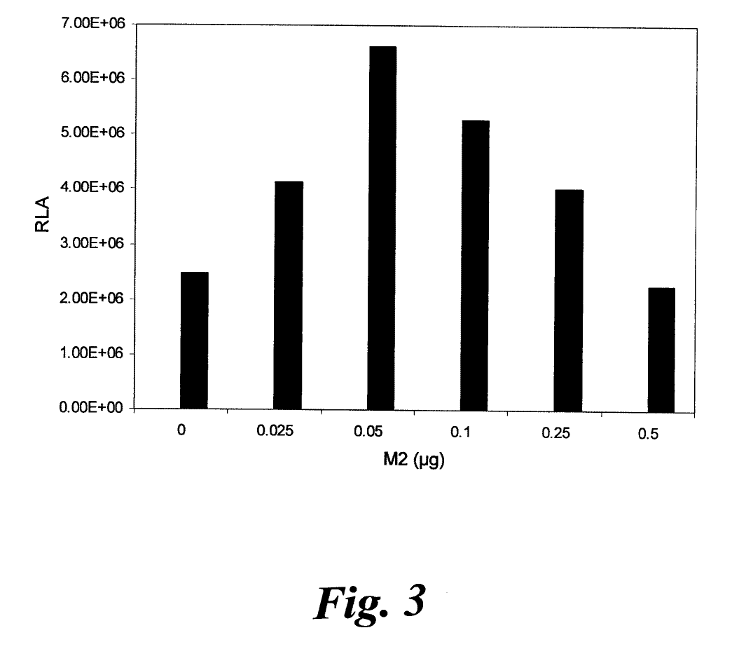

Also, co-transfection M2 with HA and NA at the optimal ratio results in a further, but moderate (2-to-3 folds) increase in transduction efficiency (FIG. 3). This 2-to-3 fold increase by M2 in our studies is much lower than the 30 fold increase in past studies (11). This difference could again be explained by the difference in dependency of M2 for PFV H7HA and H5HA during the biogenesis in packaging cells (27). However, the reason for much greater enhancement (close to 4 log) by NA in the present invention compared to past studies (e.g., 25-fold) in transduction efficiency of HA pseudotyped lentiviral vectors is not clear. In their studies, most NA effect was found with soluble bacterial NA protein from Vibrio Cholerae, although in some experiments co-transfection of influenza NA from A/PR/8/34 was also tested but the effect seen in the current application was not observed.

The NA gene was derived from a H5N1 avian flu strain and codon-optimized. While not being bound to a particular mechanism of action, one possible explanation therefore is that the dramatic enhancement of NA in transduction efficiency of H5HA pseudotyped lentiviral vector is the uniqueness of NA derived from a highly pathogenic avian flu strain.

In particular H5HA/NA/M2 pseudotypes with such enhanced transduction efficiency will have many basic science and clinical applications.

First, as a gene delivery system it can efficiently transduce epithelial cells through apical membrane (11). Therefore, likely it can be used to directly introduce genes into mucosal epithelia in vivo.

Second, it can be used as a tool of basic research to uncover the molecular mechanism of virus entry, thereby potential pathogenesis.

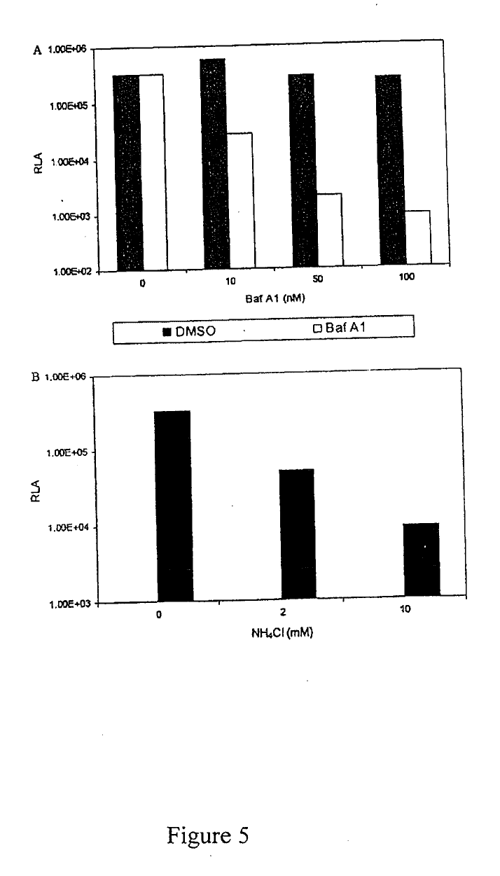

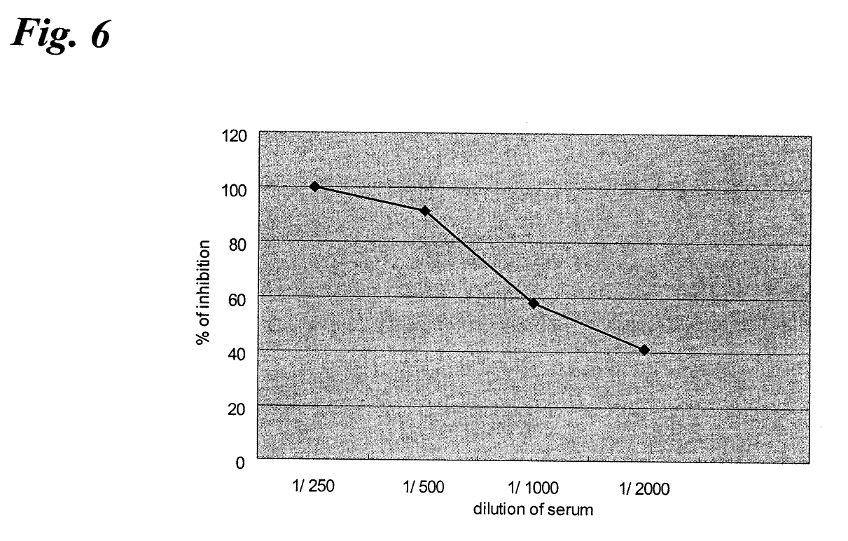

And finally, well preserved receptor binding site and antigenic determinants in H5HA/NA/M2 pseudotypes demonstrated in this study (FIGS. 5 and 6) show that H5HA/NA/M2 pseudotypes can be used to develop a high through-put assay to comprehensively study immune status of H5N1 vaccinated and infected individuals and to screen entry blockers, thereby new anti-viral drugs.

Preferably the lentivirus vector further comprising a transgene.

Such a transgene can be used as an additional marker if it is an appropriate reporter gene such as one of the various forms of Green Fluorescent Protein (GFP) or luciferase. Alternatively, the transgene can be a selectable marker such as one which confers resistance to a particular substance such as Kanamycin. Or such a transgene can be a gene product of interest which it is desired to introduce into the target cell. In particular this may be an anti-viral gene, which it is desired to investigate its effects upon the pseudotyped lentivirus.

Preferably the polynucleotide expressing HA has been codon-optimized for a target or host cell.

Such a codon optimized HA ensures optimum levels of expression in the target or host cell. Different organisms have particular biases in the codons they use most commonly to specify the various amino acid residues of a particular peptide. By modifying the coding sequence such that it uses the preferred codons of the target or host cell this ensures better and more consistent levels of expression.

Preferably the polynucleotides expressing HA and NA, and M2 if present, have been codon-optimized for a target or host cell and may be slightly different from the nucleic acid sequences listed in the sequence listing herewith annexed, provided that they are able to express the concerned proteins.

In the current invention the pseudotyped lentiviral vectors are not limited to specific HA, NA and M2 protein sequences but instead the inventors have shown that combinations of HA, NA and M2 proteins from the same or different viral isolates or indeed isolates from different HA or NA groups when used in a single pseudotyped lentiviral vector can have the required biological and immunogenic properties.

Preferably the HA protein consists of at least two portions from different HA homologues.

Preferably the NA protein consists of at least two portions from different NA homologues.

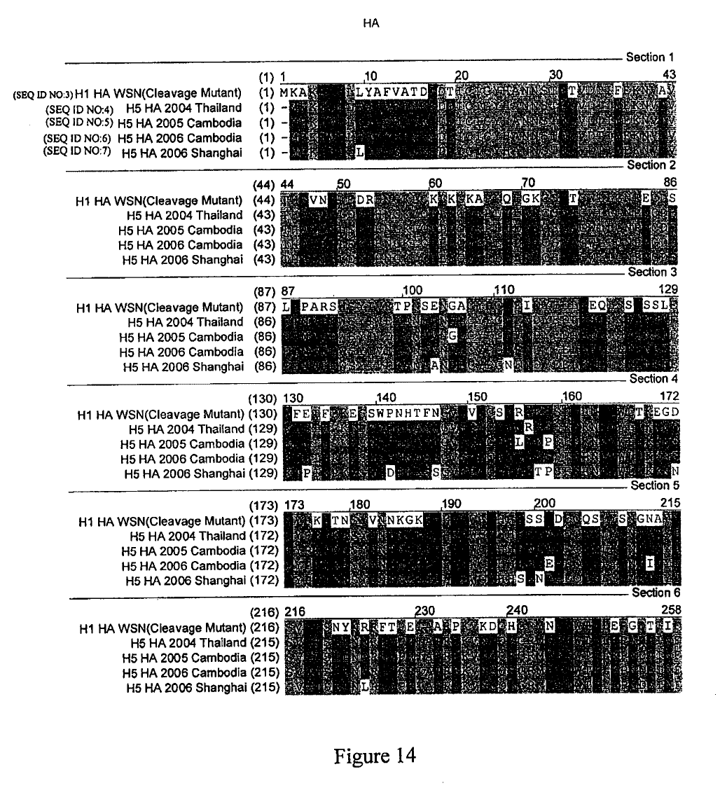

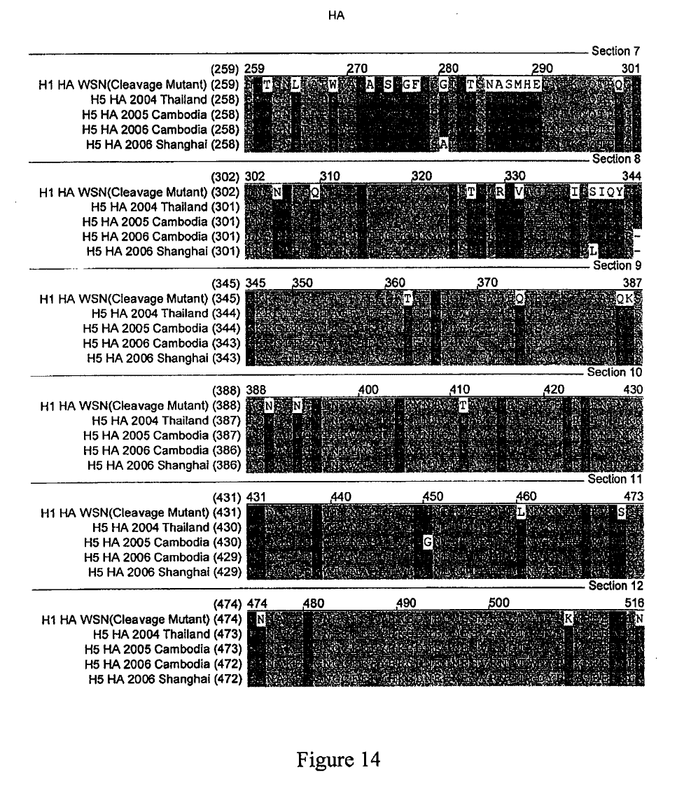

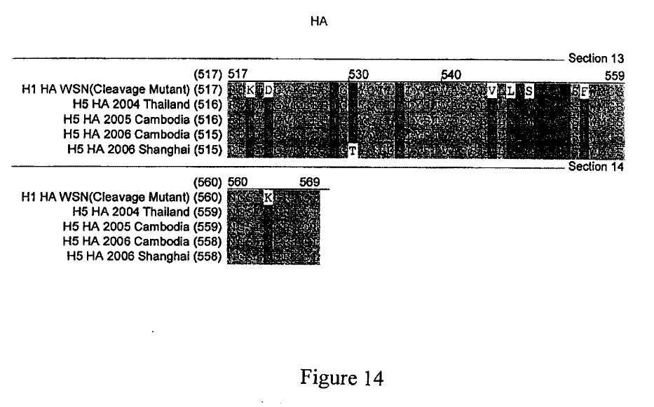

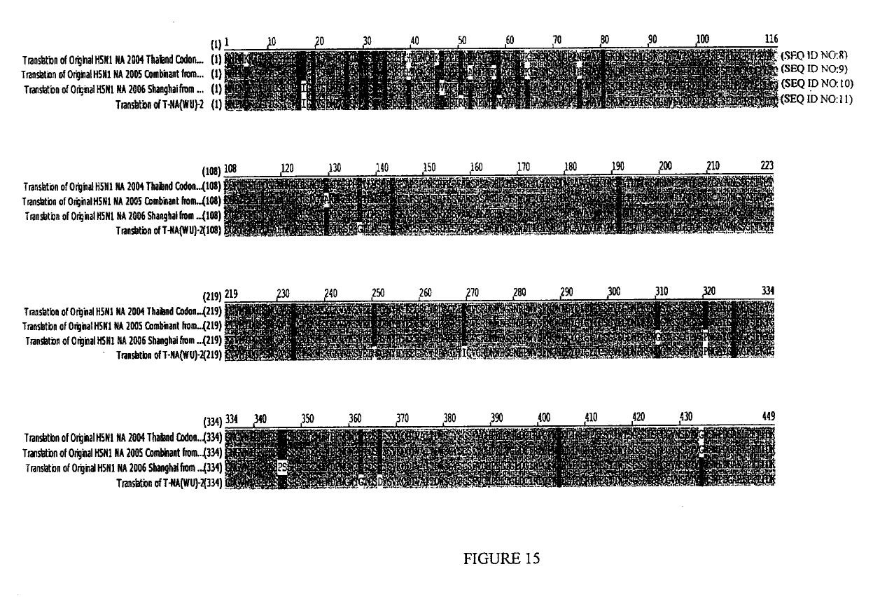

The inventors have found that by using proteins which comprise portions of at least two native proteins that these chimeric HA or NA proteins are both imunnogenic to levels comparable with the originating proteins and have biological activity. To do this the inventors have found that by combining portions or domains of different HA or NA proteins which are separated by conserved residues as can be identified with reference to FIGS. 14 and 15 herein.

There is also provided a composition comprising the lentivirus vector of the current invention and a pharmaceutically acceptable excipient, carrier and/or immunological adjuvant.

There is also provided a lentivirus vector packaging system comprising:
at least one packaging vector expressing HA, and
a transfer vector construct comprising production and packaging sequences, sequences expressing the Gag and Pol lentivirus proteins, and optionally a transgene,

wherein said HA protein is not fowl plague virus H7HA.

There is also provided a lentivirus vector packaging system comprising:
at least one packaging vector expressing HA,
a helper construct expressing the Gag and Pol lentivirus proteins, and
a transfer vector construct comprising production and packaging sequences and optionally a transgene;

wherein said HA protein is not fowl plague virus H7HA.

A lentivirus vector packaging system containing at least one packaging vector expressing HA, and a transfer vector construct comprising production and packaging sequences, sequences expressing the Gag and Pol lentivirus proteins, and optionally a transgene is contemplated. Such a packaging system may also contain at least one packaging vector expressing HA, a helper construct expressing the Gag and Pol lentivirus proteins, and a transfer vector construct comprising production and packaging sequences and optionally a transgene. Preferably, the vector does not express fowl plague virus H7HA. A target or host cell transfected with the lentivirus vector described above is also contemplated as are target or host cells transfected with the lentivirus vector of the invention which have a transgene incorporated into the chromosomal DNA. A polynucleotide sequence or a transgene may be transduced into a cell by contacting it with the lentivirus vector of the invention.

There is also provided a method for inducing an immune response comprising administering a lentivirus vector as defined hereabove to a subject in an amount sufficient to induce an immune response to said vector.

Another embodiment of the invention constitutes a method for inducing an immune response comprising administering a lentivirus vector (or a host cell transfected with it) as described above to a subject in an amount sufficient to induce an immune response. Such an immune response may be a cellular or humoral response to the pseudotyped lentivirus, such as to the HA component.

There is also provided a method for identifying a neutralizing antibody comprising:

contacting the lentivirus vector as defined hereabove with an antibody for a time and under conditions suitable for binding of the antibody to the lentivirus vector, and

determining the effects of said contact on the ability of said lentivirus vector to bind to or infect a host cell.

The H5HA/NA/M2 pseudotypes with such high efficiency have been developed by the inventors into a high through-put assay to evaluate neutralizing antibody response and to screen entry blockers of H5N1 avian flu virus.

Specific applications of this technology include the following. A lentivirus vector pseudotyped with an influenza HA protein or a protein containing an HA protein fragment comprising an HA epitope or an HA cellular attachment ligand. Preferably, the HA (or other genes, such as those for NA or M2) will be codon-optimized for a particular target or host cell. Codon-optimization is well known in the molecular biological arts. Most preferably, the lentivirus vector is not pseudotyped with fowl plague virus H7HA and the HA protein is H1, H2, H5 or H7 preferably H1, H2 or H5. However, such a lentivirus vector may also be pseudotyped with NA, such as NA from an H5N1 avian flu strain. Preferably, the vector may be pseudotyped using homologous influenza HA and NA pairing which has been discovered to be more efficient for pseudotyping a lentiviral vector. The vector may also encompass both NA and M2 as well as HA. A lentivirus vector pseudotyped with an influenza HA protein or HA protein fragment may encompass a transgene. The pseudotyped lentivirus vector may be admixed or suspended in a suitable buffer or medium, or mixed with a carrier or immunological adjuvant. Adjuvants for promoting immune responses, such as alum, Freunds incomplete or complete adjuvant, Ribi adjuvant and others are well-known in the immunological arts.

There is also provided a target or host cell transfected with the lentivirus vector of the current invention.

There is also provided a composition comprising the target or host cell of the current invention and a pharmaceutical acceptable excipient, carrier and/or immunological adjuvant.

Preferably the target or host cell transfected with the lentivirus vector of the current invention, wherein said transgene has been incorporated into the chromosomal DNA of said cell.

There is also provided a method for transducing a polynucleotide sequence or a transgene into a cell comprising contacting a cell with the lentivirus vector of the current invention for a time and under conditions sufficient for transduction.

There is also provided a method for identifying a molecule that modulates virus binding to a cell or which modulates viral infection of a cell comprising:

contacting a cell with a candidate molecule and the pseudotyped lentivirus of the current invention and

determining the ability of said candidate molecule to modulate virus binding to the cell or to inhibit viral infection of the cell.

The pseudotyped lentivirus vectors described above may be used to identify or characterize a neutralizing antibody by contacting the lentivirus vector with an antibody for a time and under conditions suitable for binding of the antibody to the lentivirus vector, and determining the effects of said contact on the ability of said lentivirus vector to bind to or infect a host cell. Another aspect of the invention is directed to identification or characterization of molecules which modulate, e.g., increase or decrease, virus binding to a cell, or molecules which attenuate (or in some cases promote) viral infection.

Such a method may include the steps of

contacting a cell with a candidate molecule and the pseudotyped lentivirus of the invention, and then determining the ability of a candidate molecule to modulate virus binding to said cell or to inhibit viral infection of the cell. Molecules to be tested in such a method include, but are not limited to, non-protein drugs, peptides or polypeptides which are not antibodies, antibodies or antibody fragments, carbohydrates, lipids, and other pharmacological substances and drugs, including both organic and inorganic agents. Preferably the molecule is a non-protein drug.

Alternatively the molecule is a peptide or polypeptide which is not an antibody.

Most preferably the molecule is an antibody.

Most preferably the molecule comprises a carbohydrate or lipid.

In accordance with a further aspect of the present invention there is provided a pseudotyped Lentivirus vector based neutralization assay comprising the steps of:

a) bringing into contact a first population of cells with
at least one Lentivirus vector comprising a marker and pseudotyped with one or more antigens selected from the group: an influenza HA protein or a protein containing an HA protein fragment comprising an HA epitope or an HA cellular attachment ligand and an influenza NA protein, a protein containing an NA protein fragment comprising an NA epitope or an NA cellular attachment ligand and
a sample of sera;

b) incubating said first population of cells with said at least one pseudotyped Lentivirus vector and said sera;

c) determining the presence of said marker in said population of cells.

Neutralizing antibody responses are critical for virus prevention and clearance and for serodiagnosis. Currently two assays are used to monitor the presence of neutralizing antibodies in a sample, the Microneutralization (MN) and the hemagglutination inhibition (HI) assay. These assays are currently used to evaluate neutralizing antibody responses against highly pathogenic avian influenza (HPAI) H5N1 viruses. However, due to the use of replication competent HPAI viruses both assays require biosafety level 3 (BSL-3) containment facilities.

The MN assay is somewhat labor intensive and HI assay is only a surrogate assay. Therefore, a neutralization assay that does not require BSL-3 facilities would be advantageous. Toward this goal, the inventors generated an influenza HA and NA pseudotype panel. Using this panel they have developed a HA/NA pseudotype-based neutralization (PN) assay comprising the steps set out above.

The inventors have now demonstrated that the HA/NA Lentivirus vector pseudotypes mimic wild type influenza A virus in their release and entry and that the PN assay according to the present invention exhibits specificity for H5N1 virus of several clades and subclades. Moreover, the inventors have demonstrated the excellent correlation between neutralization titers measured by PN assay and neutralization titers assayed by MN and HI techniques.

This pseudotyped neutralization assay therefore allows the properties of the antibodies present in a sera sample to be determined against native influenza HA and/or NA but as these antigens are presented/tested upon a Lentivirus vector rather than upon the native influenza virus, a class 3 biological containment facility is not required to perform this assay.

There are many potential applications for the PN assay other than measuring anti-H5HA neutralizing antibody responses in immune and infected sera and other bodily fluids. For example, the assay could be adapted for use in screening drug candidates that block entry and release of H5N1 viruses. It could also be used to screen anti-H5HA human and mouse monoclonal antibodies. Finally, it can be adapted for use as a tool to map neutralization epitopes by constructing pseudotypes expressing chimeras and site-directed and randomized mutants of H5HA (15).

In accordance with the present invention, a marker refers to any nucleic acid, peptide or other chemical entity which can be incorporated into the Lentivirus vector and which following transduction into the host cell can be detected either in situ or following cell lysis. Examples of markers include nucleic acid sequences coding for enzymes such as luciferase which following transduction are expressed and can be detected as a luminescent signal. Alternatively the nucleic acid can encode a detectable antigen such as FLAG or it may encode a resistance gene such as Puromycin resistance. Alternatively the marker may be a peptide, lipopeptide or other molecule which is incorporated into the Lentivirus vector and hence released into the target cell following transduction, an example of this would be a Vpr fusion protein (70).

In accordance with the present invention, sera refers to an untreated, partially purified or purified sample taken from the fluid part of a blood sample of an animal or patient. In particular the sera may have been purified so as to increase the concentration of antibodies in comparison to other components present therein.

The measurement of neutralizing antibody responses may be used for influenza serodiagnosis or as a means to determine whether protective immunity results from the evaluation of candidate pandemic influenza vaccines. The development of effective immunogens that elicit neutralizing antibody responses against genetically diverse strains of HPAI H5N1 viruses requires both the identification of appropriate HA and NA antigenic structures (39) and the identification of epitopes that induce protective antibodies (40), Moreover, the development of candidate H5N1 pandemic vaccines influenza vaccines requires for standardized in vitro assays that will allow for a meaningful comparison of the potency and the breadth of neutralizing antibody responses in sera or other body fluids from HPAI H5N1 vaccinated subjects.

The inventors also found an excellent correlation between neutralization titers measured by the MN and PN assays. Moreover, the PN assay was found to be somewhat more sensitive assay the MN or HI assay, since it was able to detect low level specific antibody responses that the other two assays could not (Tables III and IV). Finally, the inventors used sera from ferrets immunized with split virion H5N1 vaccines to show the PN assay to be a sensitive and quantifiable assay for measuring neutralizing antibody responses against diverse H5N1 clades and subclades. In these experiments, the inventors compared antibody responses measured using the PN assay to those measured by the MN and HI assays (Table IV and V).

In addition to the advantages in sensitivity and specificity demonstrated in this study, the PN assay has several advantages over the MN and HI assays for measuring antibody responses against HPAI H5N1 viruses (Table VI). Firstly, pseudotype particles undergo a single-round of replication in the indicator cell line and do not produce infectious progeny viruses. Therefore, the PN assay does not require BSL-3 facilities. Secondly, unlike HI assay the PN assay directly measures the effect of neutralizing antibody on virus entry into the cell. And thirdly, the MN assay takes 5-6 days to complete; while the PN assay can be conducted in as few as 2-3 days.

Additionally, these results illustrate the importance of choosing appropriate pseudotype doses for measuring neutralizing antibody responses against HPAI H5N1 viruses by PN assay (Table III). For use in evaluation of neutralization antibody titers elicited with vaccine candidates higher input doses of HA and NA pseudotypes that can make a meaningful comparison with 100 TCID₅₀ used in standard MNA should be used in PN assay, so that neutralization titers of immune sera or other body fluids measured by PN assay will not be overestimated. In contrast, for use in serodiagnosis, lower input doses of HA and NA pseudotypes may be used, which serves to increase the sensitivity of the assay without sacrificing its specificity.

This new pseudotyped neutralization (PN) assay therefore has several important advantages over the prior art.

In particular the Pseudotyped Lentivirus vectors have been pseudotyped with a panel of different HA and/or NA antigens.

In particular the Pseudotyped Lentivirus vectors have been pseudotyped with antigens from all the known clades and subclades of H5 and N1.

The H5N1 avian influenza virus has evolved into 10 different clades based on changes in the genetic sequence analysis. Among them, clade 2 can be subdivided into five subclades (56). The HA/NA pseudotype panel in this study only consisted of H5HA from clades as well as H1HA, H2HA, H7HA and H9HA. In H1HA, the inventors have recently made pseudotypes expressing a new swine H1HA. It would be desirable to expand the panel to cover all HA from all known clade and subclades of H5N1, so that immunogenicity and cross-reactivity of hemagglutinins among diverse H5N1 strains can be studied in greater detail and to determine to what extent genetic differences correspond to differences in serotype. In addition, pseudotypes expressing other subtypes of HA and NA should also be studied, particularly in view of three recent reports showing that various anti-H5HA monoclonal antibodies generated from HPAI H5N1 infected individuals or anti-HA monoclonal antibodies generated from seasonal influenza vaccination cross reacted with HA from other influenza subtypes (61-63).

In particular the marker is selected from the group: luciferase, LacZ, an antibiotic resistance gene, a toxin resistance gene, a florescent peptide fragment or protein, a peptide fragment or tag against which an antibody has been generated.

In particular the method according to this aspect of the present invention involves the treatment of at a least a second and/or further cell populations with said pseudotyped Lentivirus vectors and sera, and wherein said marker is detected quantitatively and the detected level of said marker is averaged between said first, second and further populations.

In particular the method according to this aspect of the present invention involves comparing the detected level of said marker in cell populations exposed to said Pseudotyped Lentivirus vectors and sera to cell populations not exposed to said Pseudotyped Lentivirus vectors and/or said sera.

In particular the first, second and further cell populations consist of cells chosen from the group comprising: MDCK, CEMss, CHO, 293T, HeLa, Vero, HT-29 and Caco2 cells.

In addition the present invention also relates to a neutralization method using pseudotyped Lentivirus vectors comprising epitopes from other organisms such as viruses like vesicular stomatitis virus (VSV), hepatitis C virus (HCV), the SARS coronavirus, Ebola, avian influenza H7N7, influenza H1N1, murine leukemia virus (MLV) and the lassa fever virus (46-53) as well as bacterial and eukaryotic pathogens, comprising the steps of the method as set out above.

For a better understanding of the invention and to show how the same may be carried into effect, there will now be shown by way of example only, specific embodiments, methods and processes according to the present invention with reference to the accompanying drawings in which:

FIG. 1: shows schematic diagram of the transfer and packaging vectors as well as DNA constructs expressing H5HA, NA and M2. Cell surface expression of H5HA on 293T packaging cells transfected with mock (negative control), H5HA (1A), H5HA and M2 (1B), H5HA and NA (1C), and H5HA, NA and M2 (1D). The transfected cells were stained with pooled immune sera specific for H5HA and followed by FITC-conjugated goat anti-mouse IgG antibody.

FIG. 2: shows the relative luciferase activity (RLA) in MDCK cells transduced with supernatants derived from 293T cells transfected with mock, H5HA with or without the various indicated amounts of NA pseudotypes (2A) and in MDCK cells transduced with supernatants derived from 293T cells transfected with mock, H5HA with or without the various indicated amounts of M2 pseudotypes (2B).

FIG. 3: shows the relative luciferase activity (RLA) in MDCK cells transduced with supernatants derived from 293T cells transfected with H5HA and NA at 4:1 ratio with or without the various indicated amounts of M2.

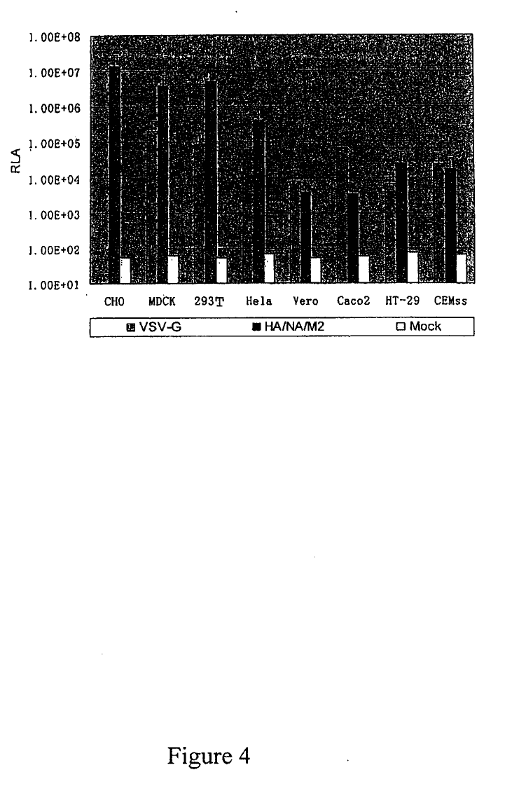

FIG. 4: shows the relative luciferase activity (RLA) in CHO, MDCK, 293 T, HeLa, Vero, Caco2, HT29 and CEMss cells transduced with mock or supernatants containing H5HA/NA/M2 or VSV-G pseudotypes equivalent to 10 ng HIV-1 gag p24. During the transfection, 293T cells were transfected with H5HA, NA, and M2 at an optimal ratio of 8:2:1.

FIG. 5: shows the relative luciferase activity (RLA) in MDCK cells pretreated with bafilomycin A1 (5a) or NH₄Cl (5b) and then transduced with supernatants containing H5HA/NA/M2 pseudotypes. During the transfection, 293T cells were transfected with H5HA, NA, and M2 at an optimal ratio of 8:2:1.

FIG. 6: shows the percentage of inhibition of transduction efficiency of H5HA/NA/M2 or VSV-G pseudotypes pretreated with either pooled preimmune or postimmune serum samples specific for H5HA.

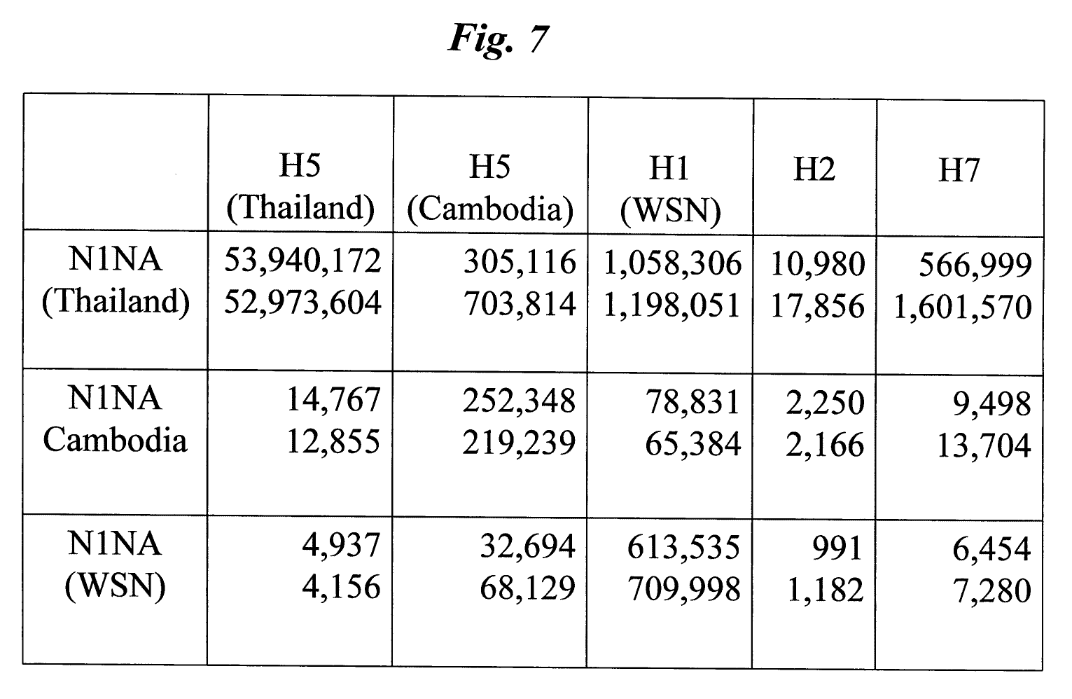

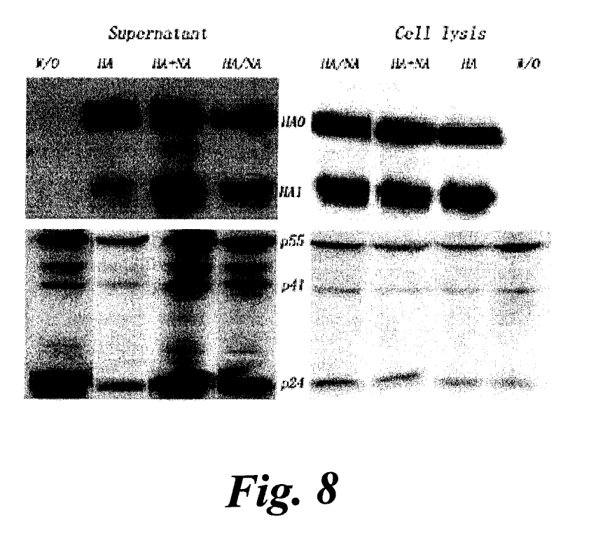

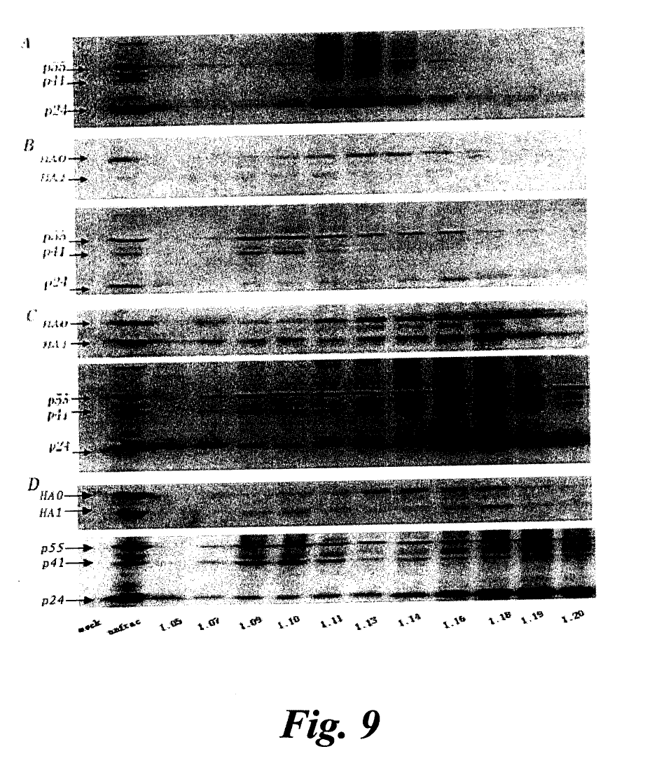

FIG. 7: shows the results of luciferase activity of lentiviral vectors pseudotyped with influenza HA and NA derived from several subtypes of avian and human viruses. Homologous influenza HA and NA pairing is more efficient for pseudotyping lentiviral vectors and this finding has implications for the further investigation and use of influenza viruses.

FIG. 8. shows a western of supernatant and cell lysate from cells transfected with H5HA alone with or without exogenous NA treatment or co-transfected with H5HA/NA.

FIG. 9. shows a western upon isolated fractions from cells transfected with H5HA alone with or without exogenous NA treatment or co-transfected with H5HA/NA in which, panel A is untransfected cells, panel B is cells transfected with H5HA alone, panel C is cells co-transfected with H5HA/NA and panel D is cells transfected with H5HA treated with exogenous NA treatment.

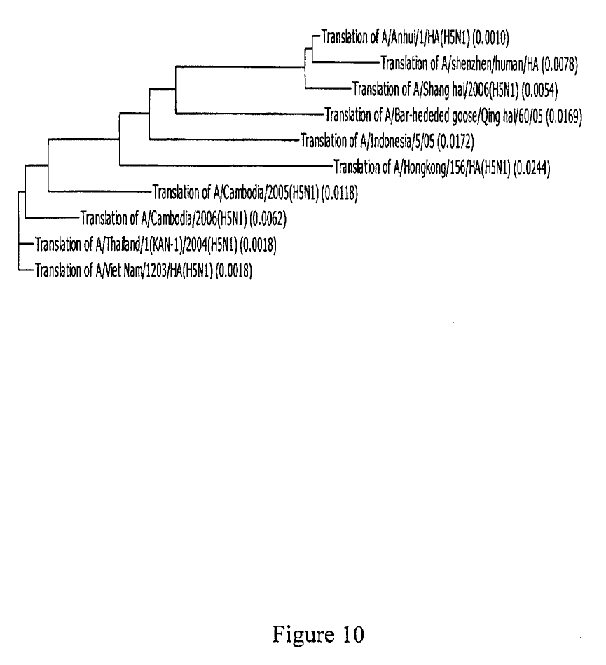

FIG. 10. shows the phylogeny of H5HA and the various subclades thereof.

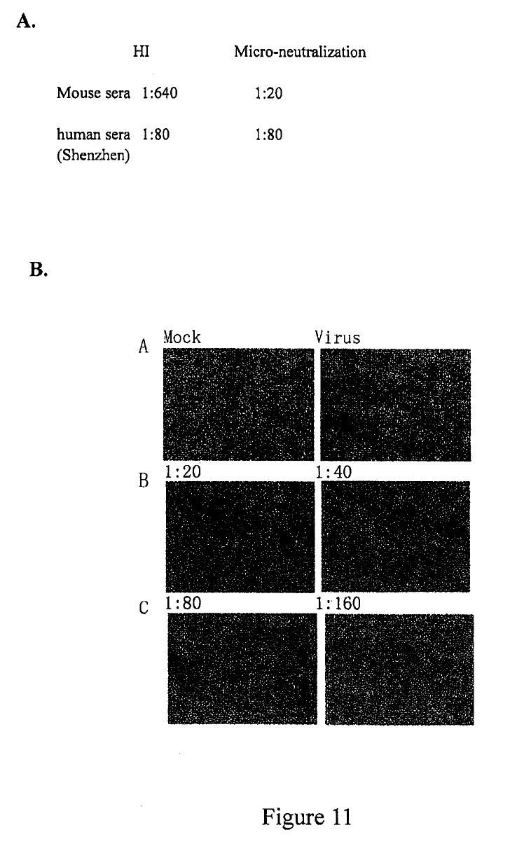

FIG. 11. shows the results of the HI (FIG. 11A) and microneutralization assay (FIG. 11B) performed as a comparison to the new H5HA/NA assay upon anti-H5HA (subclade 1.1) mouse sera and convalescent human sera for H5N1 (subclade 2.3).

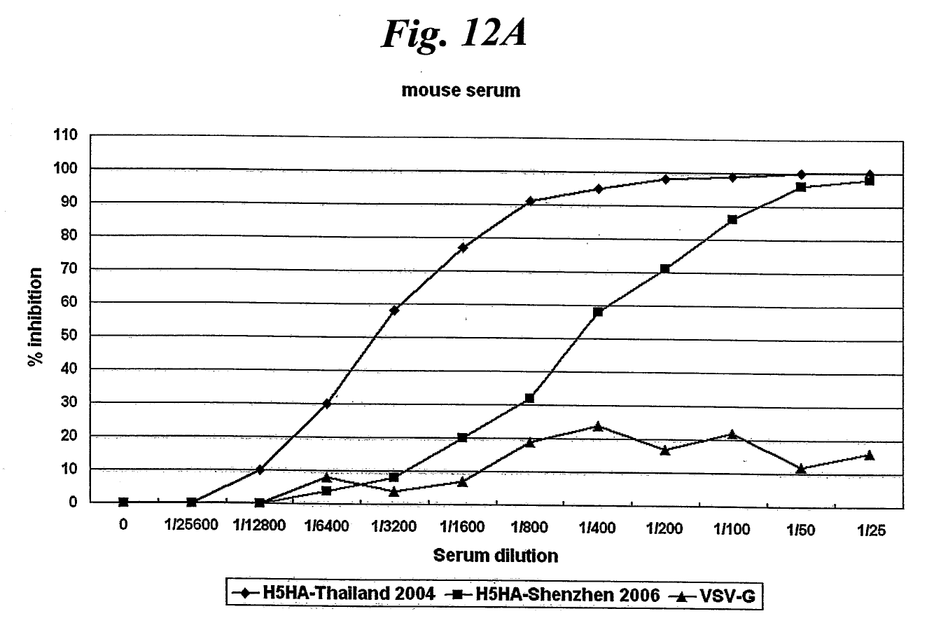

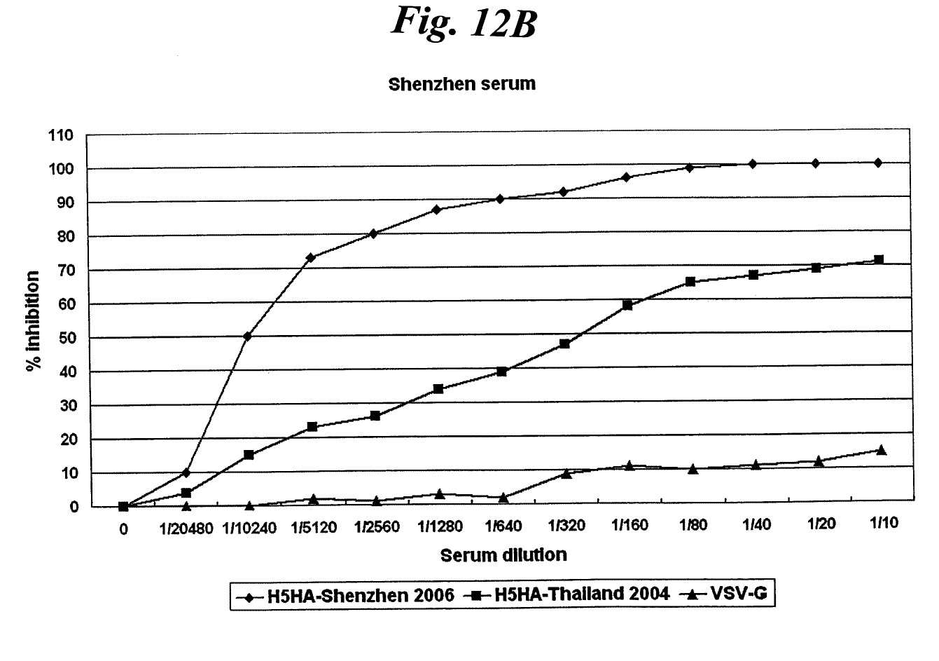

FIG. 12. shows the results of the new H5HA/NA assay upon anti-HA (subclade 1.1) mouse sera and the results of the new H5HA/NA assay upon convalescent human sera for H5N1 (subclade 2.3).

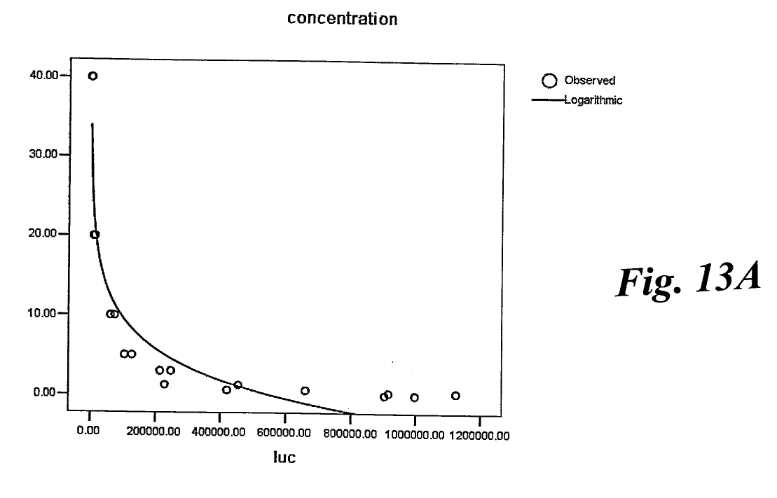

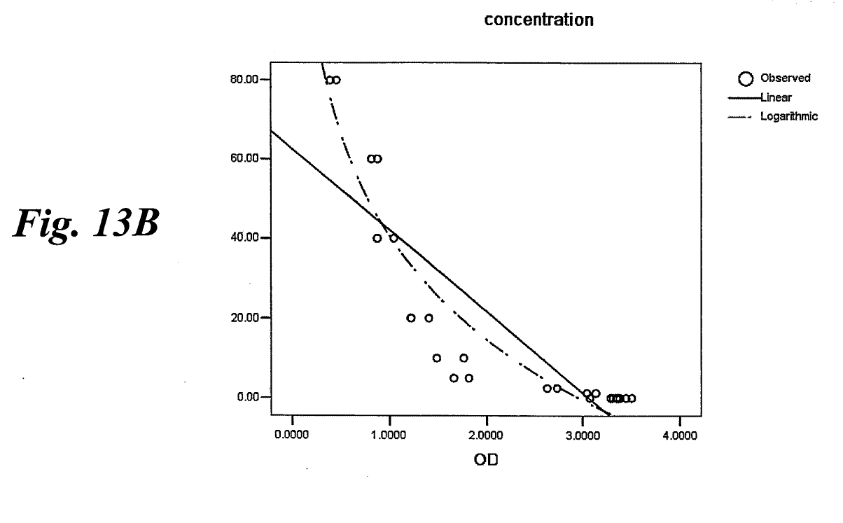

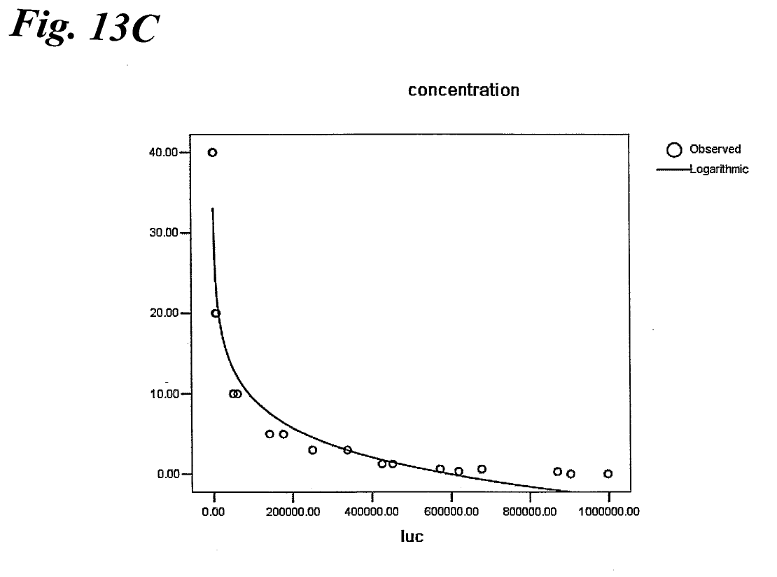

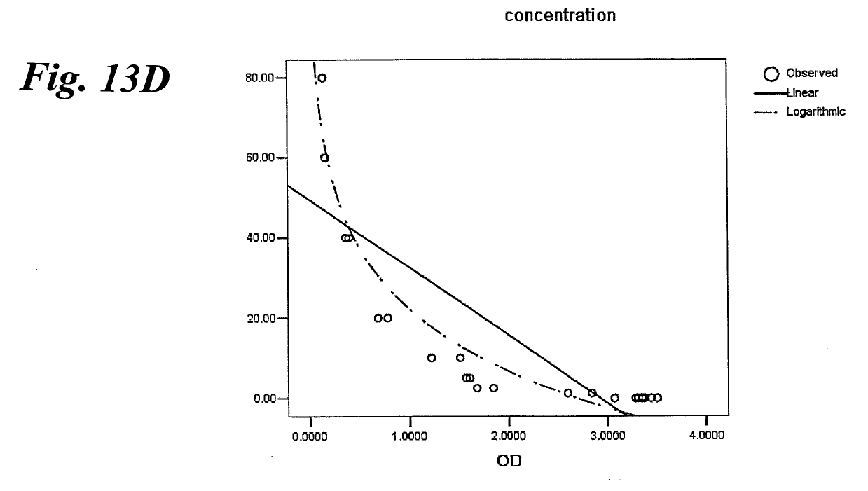

FIG. 13. shows how the EC50 (13A and 13C) and the CC50 (13B and 13D) of two compounds isolated from several traditional Chinese herbs by HPLC (compound 1: FIGS. 13A and B; compound 2: FIGS. 13C and D).

FIG. 14. shows a peptide sequence comparison of the H1HA sequence with mutations to create multibasic site and H5MA sequences of different strains.

FIG. 15. shows a peptide sequence comparison of different NA sequences.

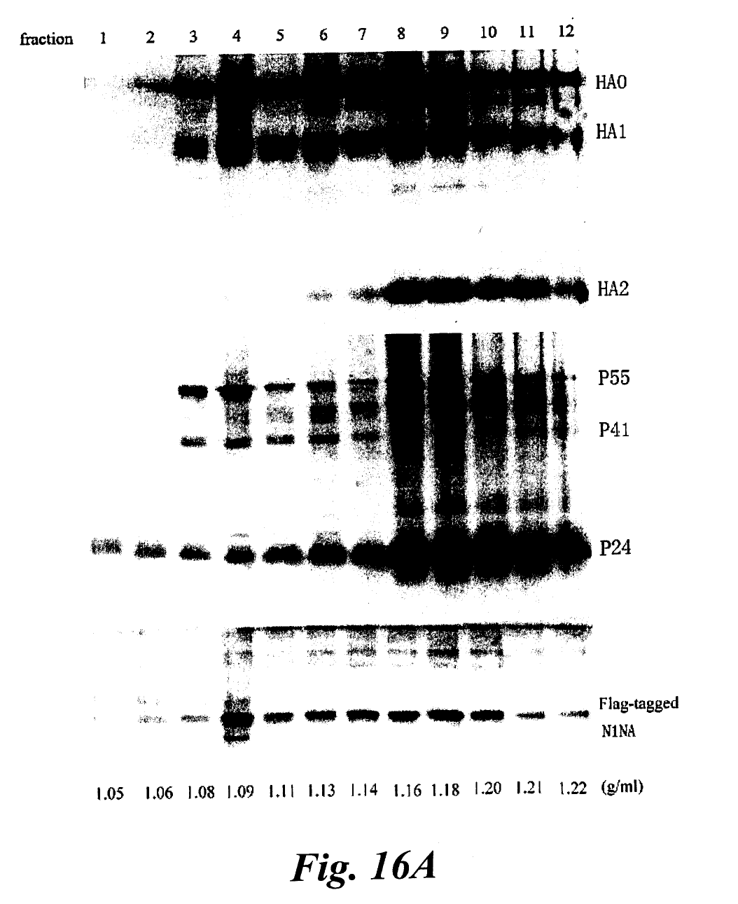

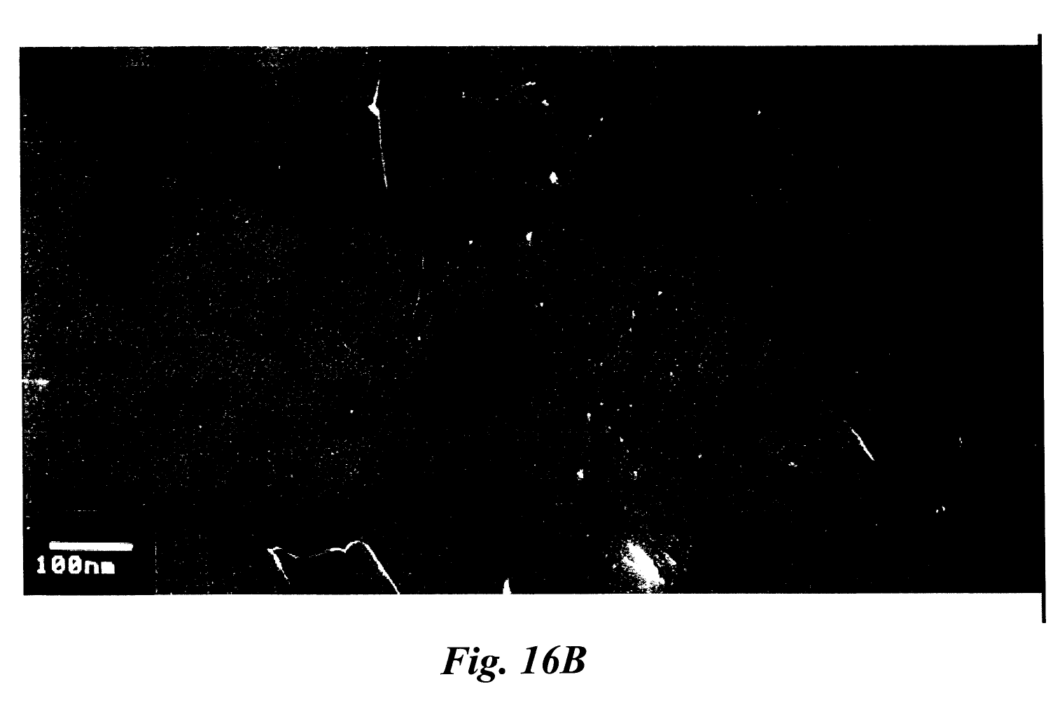

FIG. 16. Characterization of influenza H5HA and N1NA pseudotypes. (a) Western blot analysis of influenza HA, NA and HIV-1 gag proteins in 12 fractions after sucrose gradient fractionation detected by anti-H5HA-specific immune sera, anti-flag epitope antibody and anti-HIV-1 gag p24 antibody. (b) H5HA and N1NA pseudotypes that are being formed on the cell surface and released from 239 T packaging cells revealed by electron microscopy. (c) Comparison of transduction efficiency in target MDCK cells transduced with mock, vector alone (transfer vector and packaging vector alone), HA alone (transfer vector, packaging vector and plasmid encoding H5HA), HA alone plus exogenous NA treatment, and HA and NA (transfer vector, packaging vector and plasmids encoding H5HA and N1NA). RLA: relative luciferase activity. (d-e) Effect of pretreatment of target Maji-CCR5 cells with indicated doses of NH₄Cl (d) or Bafilomycin A1 (e) on transduction efficiency of HA and NA and HIV-1 envelope Ad8 pseudotypes. (f-g) Effect of treatment of packaging 293T cells (f) and target Maji-CCR5 cells (g) with indicated doses of neuraminidase inhibitor oseltamivir phosphate on transduction efficiency of HA and NA, HIV-1 envelope Ad8, and VSV-G pseudotypes.

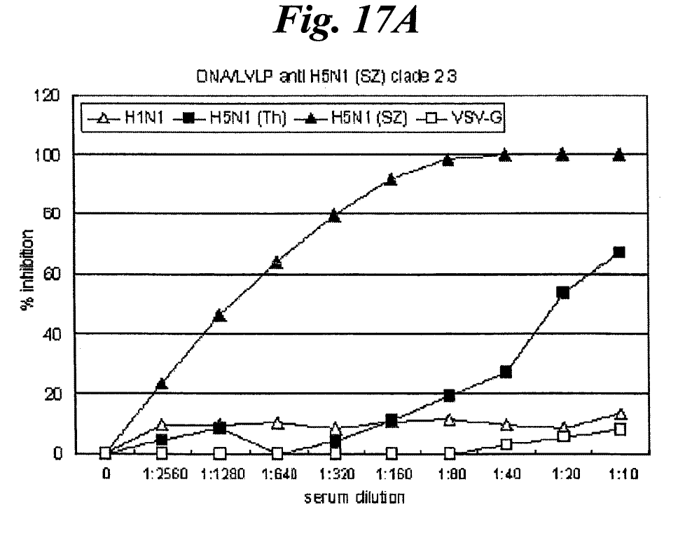

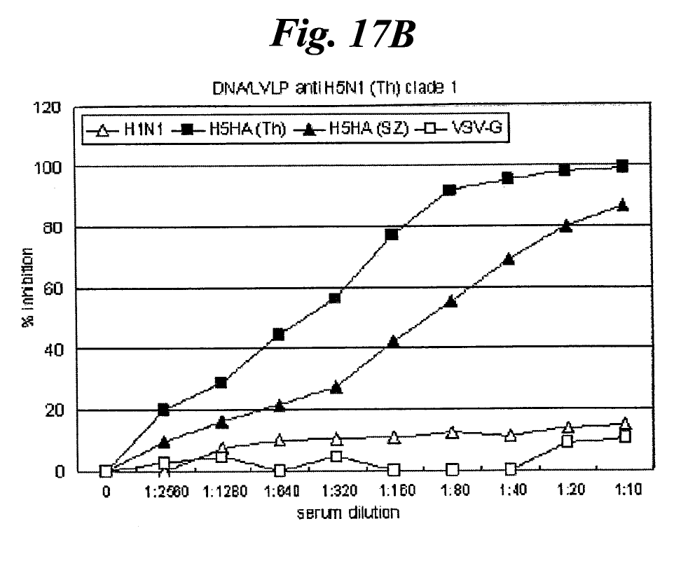

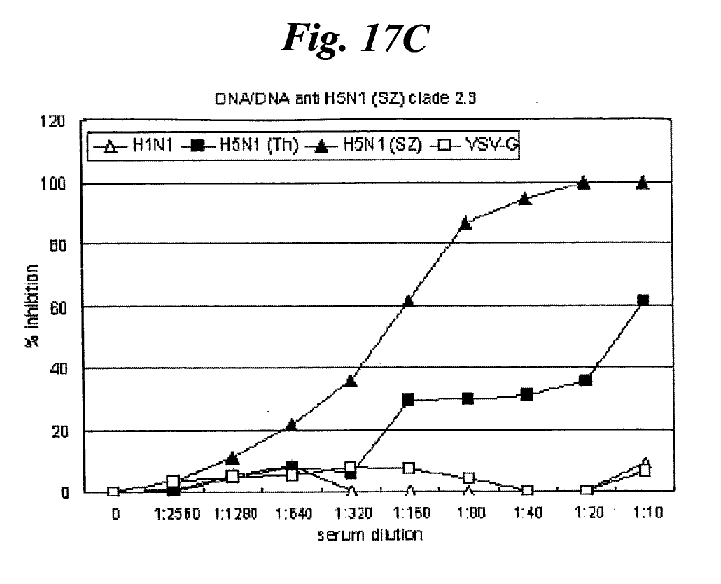

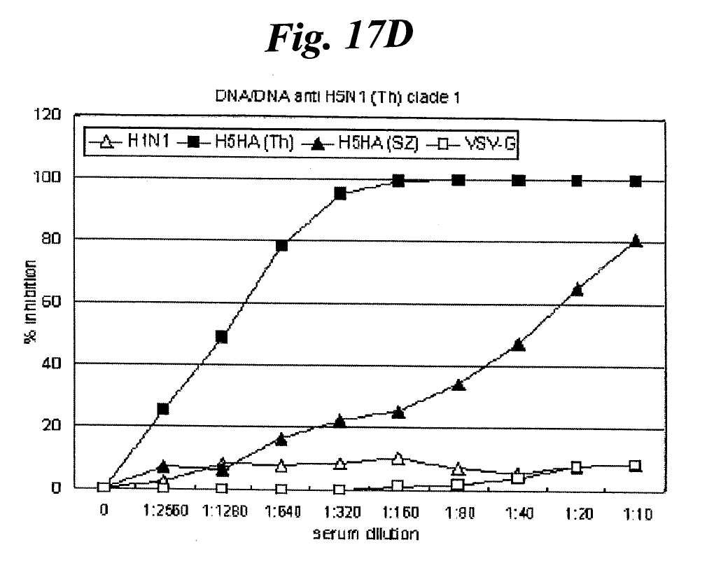

FIG. 17. Comparison of neutralization activity of immune sera against four different pseudotypes: H5HA (A/Thailand/1(KAN-1)/04, clade 1) and N1NA, H5HA (A/Shenzhen/406H/06, clade 2.3) and N1NA, H1HA (cleavage mutant of WSN) and N1NA and VSV-G control measured by PNA. (a) Immune serum elicited with priming and boosting of DNA expressing subclade 2.3H5HA (A/Shenzhen/406H/06). (b) Immune serum elicited with priming and boosting of DNA expressing clade 1H5HA (A/Thailand/1(KAN-1)/04). (c) Immune serum elicited with priming of DNA expressing subclade 2.3H5HA (A/Shenzhen/406H/06) and boosting of LVLP expressing both subclade 2.3H5HA (A/Shenzhen/406H/06) and N1NA. (d) Immune serum elicited with priming of DNA expressing clade 1H5HA (A/Thailand/1(KAN-1)/04) and boosting of LVLP expressing both clade 1H5HA (A/Thailand/1(KAN-1)/04) and N1NA.

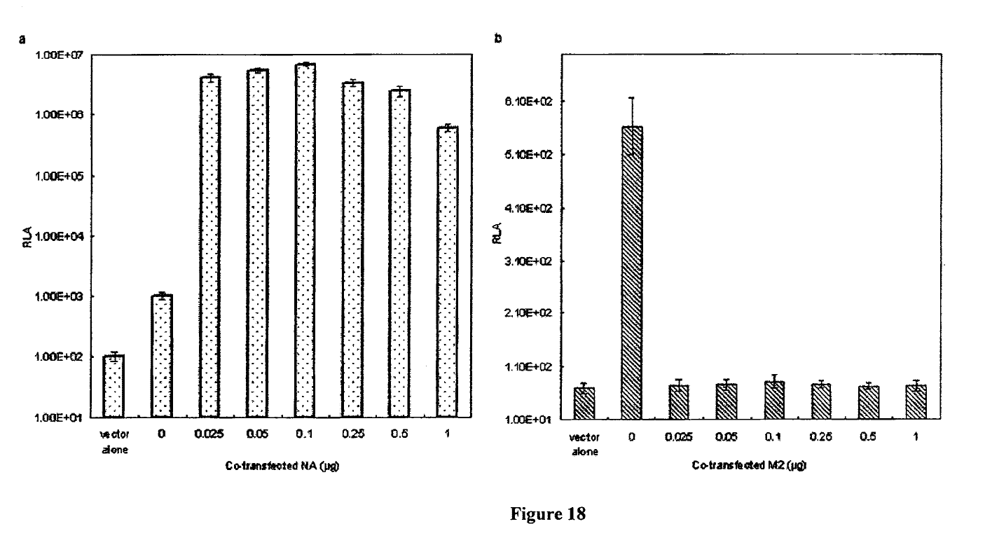

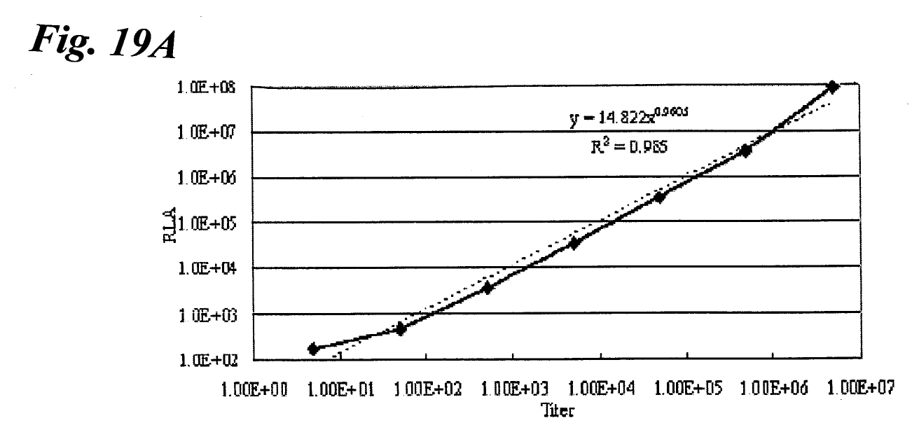

FIG. 18. Effect of co-transfected influenza NA or M2 on transduction efficiency of H5HA-pseudotyped lentiviral vector. (a) Transduction efficiency of H5HA-pseudotyped lentiviral vector with various indicated doses of co-transfected NA. (b) Transduction efficiency of H5HA-pseudotyped lentiviral vector with various indicated doses of co-transfected M2. RLA: relative luciferase activity.

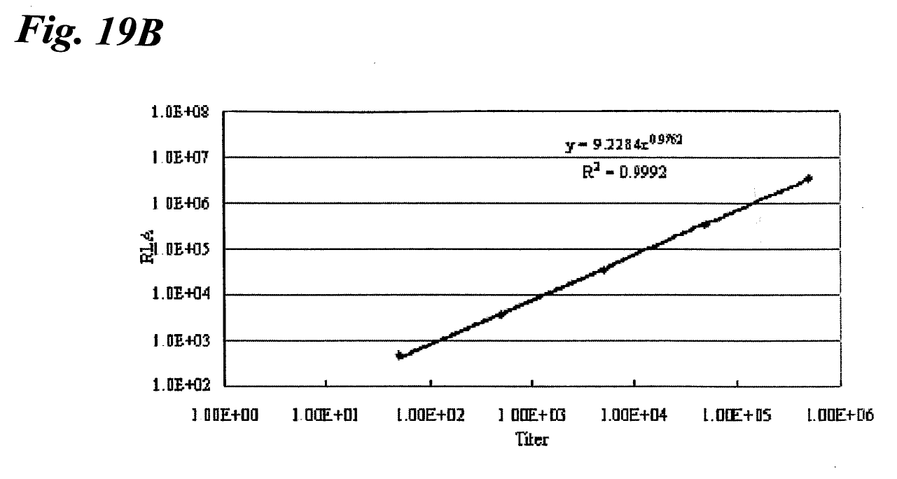

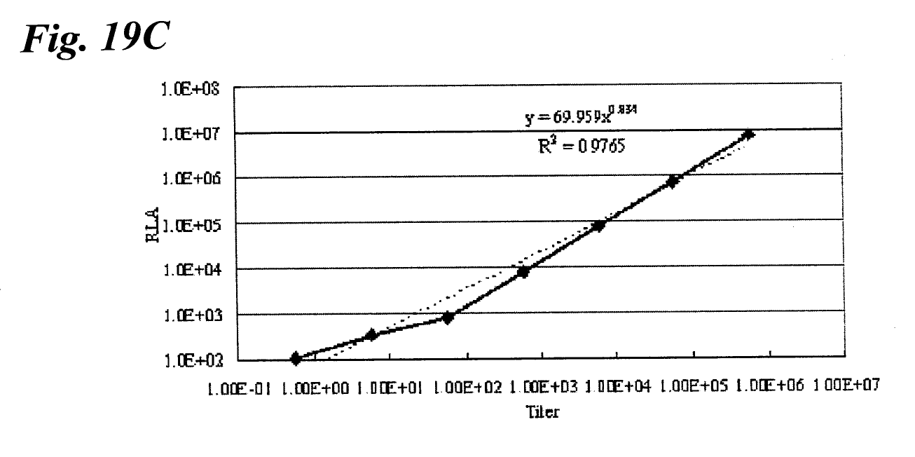

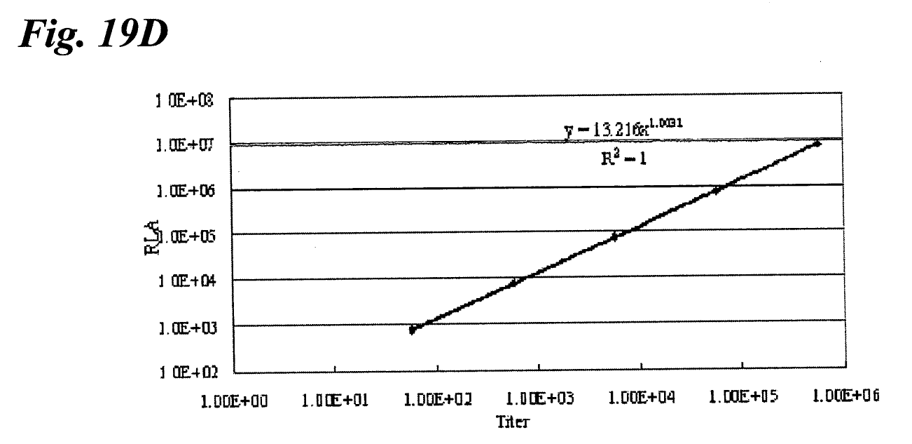

FIG. 19. Correlations between relative luciferase activity (RLA) and transducing titers of two representative HA and NA pseudotypes (A/Thailand/1(KAN-1)/04, clade 1 and A/Shenzhen/406H/06, subclade 2.3) in different dose ranges. (a) Correlation between RLA and transducing titers from 2×10⁰ to 2×10⁶ of HA and NA pseudotypes (A/Thailand/1(KAN-1)/04). (b) Correlation between RLA and transducing titers from 2×10² to 2×10⁵ of HA and NA pseudotypes (A/Thailand/1(KAN-1)/04). (c) Correlation between RLA and transducing titers from 2×10⁰ to 2×10⁶ of HA and NA pseudotypes (A/Shenzhen/406H/06). (d) Correlation between RLA and transducing titers from 2×10¹ to 2×10⁵ of HA and NA pseudotypes (A/Shenzhen/406H/06).

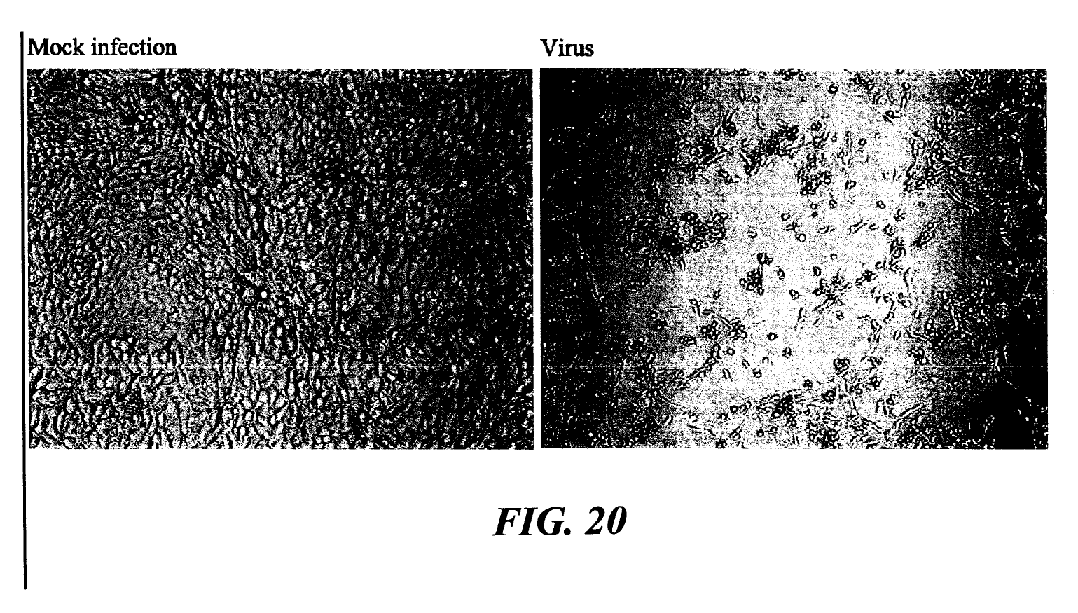

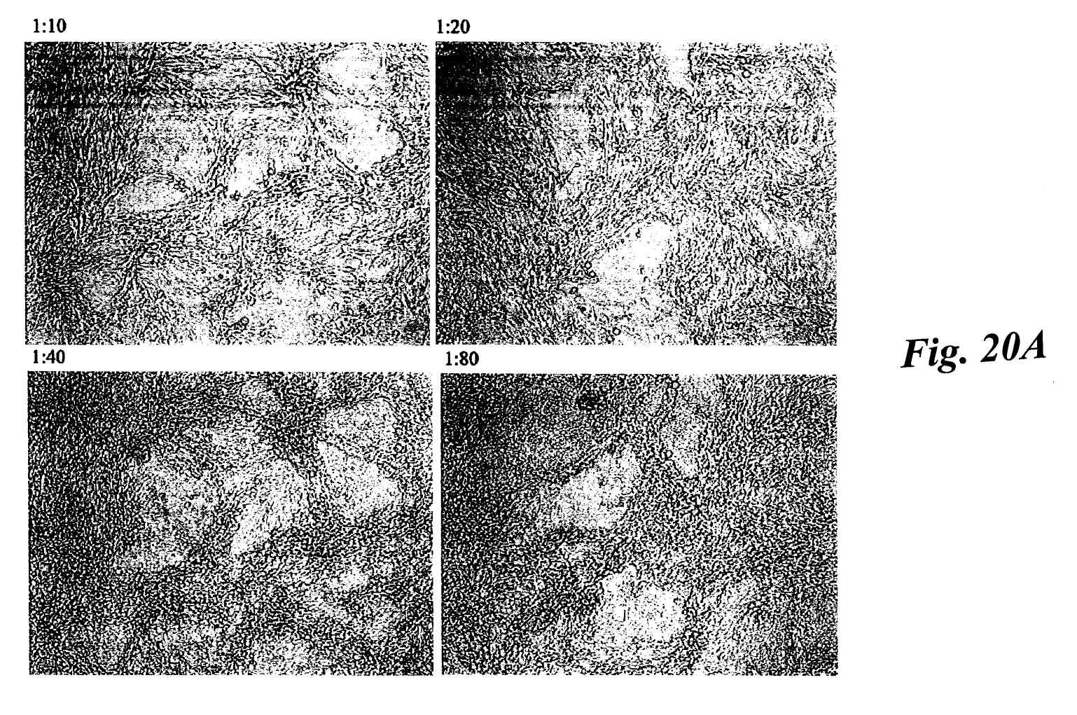

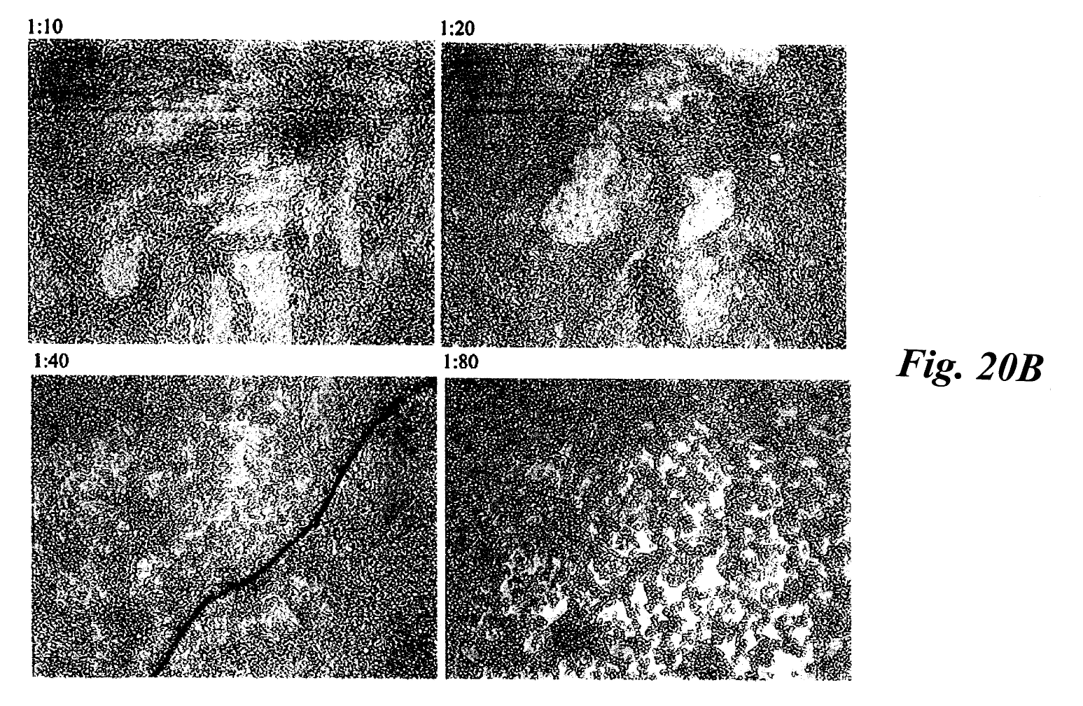

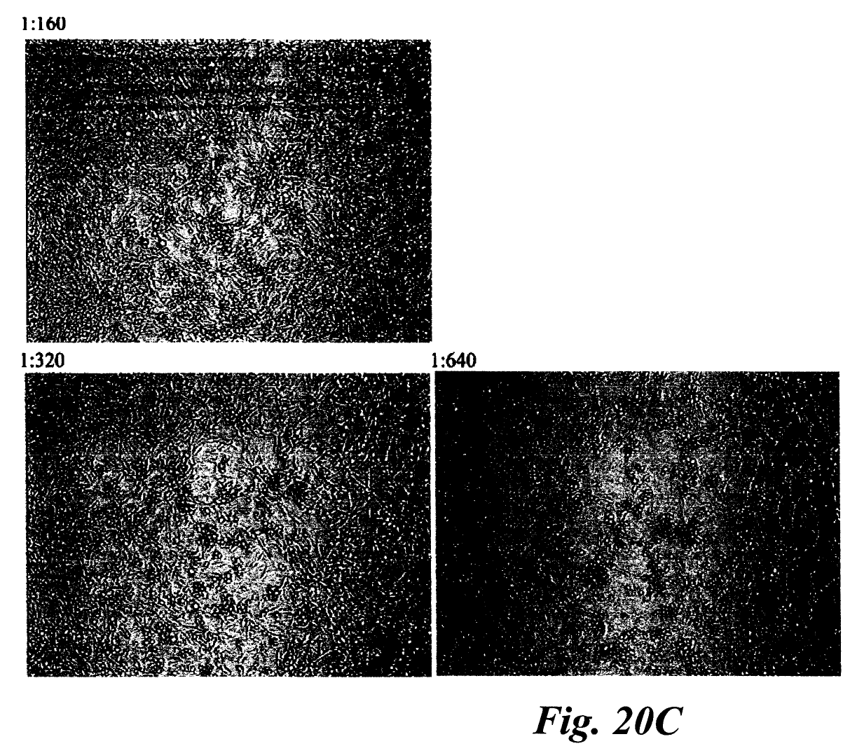

FIG. 20. Effect of various dilutions of immune sera on cytopathic effect (CPE) measured by MNA. (a) CPE shown in target cells infected with or without wild type H5N1 virus and immune sera elicited with DNA priming and LVLP boosting. (b) CPE shown in target cells infected with or without wild type H5N1 virus and immune sera elicited with DNA priming and DNA boosting. Mock: without wild type H5N1 virus (A/Shenzhen/406H/06); virus: with wild type H5N1 virus (A/Shenzhen/406H/06), but without immune sera.

The current invention also relates to a number of sequences, listed in the herewith attached sequence listing and summed up hereafter:

SEQ ID NO: 1 is the peptide sequence of H2HA

SEQ ID NO: 2 is the nucleotide coding sequence of the M gene

SEQ ID NO: 3 is the peptide sequence of H1HA WSN

SEQ ID NO: 4 is the peptide sequence of H5HA 2004 Thailand

SEQ ID NO: 5 is the peptide sequence of H5HA 2005 Cambodia

SEQ ID NO: 6 is the peptide sequence of H5HA 2006 Cambodia

SEQ ID NO: 7 is the peptide sequence of H5HA 2006 Shanghai

SEQ ID NO: 8 is the peptide sequence of H5N1 NA 2004 Thailand

SEQ ID NO: 9 is the peptide sequence of H5N1 NA 2005 Combiant

SEQ ID NO: 10 is the peptide sequence of H5N1 NA 2006 Shanghai

SEQ ID NO: 11 is the peptide sequence of T-NA(WU)-2

SEQ ID NO: 12 is the peptide sequence of H151HA

SEQ ID NO: 13 is the peptide sequence of H515HA

SEQ ID NO: 14 is the nucleotide coding sequence of H151HA

SEQ ID NO: 15 is the nucleotide coding sequence of H515HA

SEQ ID NO: 16 is the nucleotide coding sequence of H1HA WSN

SEQ ID NO: 17 is the nucleotide coding sequence of H5HA 2004 Thailand

SEQ ID NO: 18 is the nucleotide coding sequence of H2HA

SEQ ID NO: 19 is the nucleotide coding sequence of H5HA 2005 Cambodia

SEQ ID NO: 20 is the nucleotide coding sequence of H5HA 2006 Cambodia

SEQ ID NO: 21 is the nucleotide coding sequence of H5HA 2006 Shanghai

SEQ ID NO: 22 is the nucleotide coding sequence of H5N1 NA 2004 Thailand

SEQ ID NO: 23 is the nucleotide coding sequence of H5N1 NA Combiant

SEQ ID NO: 24 is the nucleotide coding sequence of H5N1 NA Shanghai

SEQ ID NO: 25 is the nucleotide coding sequence of T-NA(WU)-2

SEQ ID NO: 26 is the peptide sequence of the peptide sequence of the codon optimized NA of H5N1 human isolate A/Thailand/1(Kan-1)/04 (SEQ ID NO: 33) with a flag epitope inserted between residues 50 and 51 of the native sequence.

SEQ ID NO: 27 is the peptide sequence of the A/Anhui/1/05 isolate HA protein.

SEQ ID NO: 28 is the peptide sequence of the A/Cambodia/P0322095/05 isolate HA protein.

SEQ ID NO: 29 is the peptide sequence of the A/Cambodia/Q0321176/06 isolate HA protein.

SEQ ID NO: 30 is the peptide sequence of the A/HongKong/156/97 isolate HA protein.

SEQ ID NO: 31 is the peptide sequence of the A/Indonesia/5/05 isolate HA protein.

SEQ ID NO: 32 is the peptide sequence of the A/Shenzhen/406H/06 isolate HA protein.

SEQ ID NO: 33 is the peptide sequence of the A/Thailand/1(KAN-1)/04 isolate HA protein.

SEQ ID NO: 34 is the peptide sequence of the A/Vietnam/1203/04 isolate HA protein.

SEQ ID NO: 35 is the peptide sequence of the A/WSN/1933 (mutation) HA protein.

SEQ ID NO: 36 is the nucleotide coding sequence of the peptide sequence of the codon optimized NA of H5N1 human isolate A/Thailand/1 (Kan-1)/04 (SEQ ID NO: 33) with a flag epitope inserted between residues 50 and 51 of the native sequence.

SEQ ID NO: 37 is the nucleotide coding sequence of the A/Anhui/1/05 isolate HA protein.

SEQ ID NO: 38 is the nucleotide coding sequence of the A/Cambodia/P0322095/05 isolate HA protein.

SEQ ID NO: 39 is the nucleotide coding sequence of the A/Cambodia/Q0321176/06 isolate HA protein.

SEQ ID NO: 40 is the nucleotide coding sequence of the A/HongKong/156/97 isolate HA protein.

SEQ ID NO: 41 is the nucleotide coding sequence of the A/Indonesia/5/05 isolate HA protein.

SEQ ID NO: 42 is the nucleotide coding sequence of the A/Shenzhen/406H/06 isolate HA protein.

SEQ ID NO: 43 is the nucleotide coding sequence of the A/Thailand/1(KAN-1)/04 isolate HA protein.

SEQ ID NO: 44 is the nucleotide coding sequence of the A/Vietnam/1203/04 isolate HA protein.

SEQ ID NO: 45 is the nucleotide coding sequence of the A/WSN/1933 (mutation) HA protein.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Materials and Methods

The following materials and methods were used to obtain the data described below.

Cell lines. The packaging cell line 293T was maintained in complete Dulbecco's modified Eagle's medium (DMEM) [i.e. high glucose DMEM supplemented with 10% FBS, 2 mM L-glutamine, 1 mM sodium pyruvate, penicillin (100 U/ml),) and streptomycin (100 μg/ml); Invitrogen Life Technologies] containing 0.5 mg/ml of G418. HeLa, Vero, human CD4 T cell line CEMss, CHO, and Madin-Darby canine kidney (MDCK) cell lines were maintained in complete DMEM medium. Human epithelial cell lines HT29 and Caco-2 were purchased and maintained in complete DMEM medium. Maji-CCR5 cells, originally reported by Deng et al. (24), were obtained from NIH AIDS Research and Reference Reagent Program and maintained in complete DMEM supplemented with 0.2 mg/ml G418, 0.1 mg/ml hygromycin B and 1 μg/ml puromycin. MDCK cells were maintained in complete DMEM medium in a humidified incubator at 37° C. with 5% CO₂.

Transfer vector, packaging vector and DNA plasmids expressing codon optimized H5HA, NA and M2. Transfer vector pHR′CMV-Luc and packaging vector pCMVΔR8.2 were originally developed by Naldini et al. (36). The codon-optimized H5HA, NA, and M2 sequences of a H5N1 avian flu strain A/Hong Kong/156/97, A/Vietnam/1203/04, A/Thailand/1(KAN-1)2004 A/Anhui/1/05 and A/Indonesia/5/05 were determined using a GCG Package (Genetic Computer Group, Inc. Madison, Wis.) and generated by a recursive PCR as previously described (37) and inserted into a mammalian expression vector CMV/R derived from pNGVL-3 (38). The resulting plasmid constructs were designated as CMV/R-H5HA, CMV/R-NA and CMV/R-M2, respectively (FIG. 1A).

The inserts containing the correct H5HA sequences were recloned into a mammalian expression vector CMV/R derived from pNGVL-3 (38). The resulting plasmid constructs were designated as CMV/R-H5HAs (A/Hong Kong/156/97, A/Vietnam/1203/04, A/Thailand/1(KAN-1)/04, A/Anhui/1/05, A/Indonesia/5/05), respectively.

To isolate genes encoding HAs of H5N1 human isolates, A/Cambodia/P0322095/05, A/Cambodia/Q0321176/06 and A/Shenzhen/406H/06, viral RNA were isolated from heat-inactivated virus-containing supernatants. Complementary DNA encoding HAs were generated by RT-PCR using the same pair of HA-specific primers as described by Hoffmann et al. (67) inserted in a TA vector and sequenced. The inserts containing correct HA sequences were recloned into an expression vector CMV/R as described above. The resulting plasmid constructs were designated as CMV/R-H5HAs (A/Cambodia/P0322095/05, A/Cambodia/Q0321176/06 and A/Shenzhen/406H/06), respectively.

The HA with multibasic cleavage site mutant of a human H1N1 influenza strain A/WSN/1933 was generated by an overlapping PCR with a gene encoding the wild type of HA of WSN strain as a template (supplied by Dr. Tetseuya Toyoda of Institut Pasteur of Shanghai) and inserted in a TA vector and sequenced. The insert containing correct HA sequence was recloned into an expression vector CMV/R.

To facilitate NA detection, codon optimized NA of a H5N1 human isolate A/Thailand/1 (KAN-1)/04, in which a flag epitope (N′-DYKDDDDK-C′) (SEQ ID NO: 26) was inserted into the stalk region (between amino acid residues 50 and 51) as described by Luo et al. (68), was generated by a recursive PCR and cloned into a TA vector and sequenced. The insert containing the correct flag epitope-tagged N1NA sequence was recloned into a mammalian expression vector CMV/R. In the initial studies the inventors found similar transduction efficiency between H5HA and N1NA and H5HA and flag-tagged N1NA pseudotypes (data not shown). Thus, the inventors used the .flag-tagged N1NA in all subsequent experiments (for the sake of simplicity, in all remaining text the inventors use NA to describe flag-tagged N1NA).

Production of pseudotypes. To generate pseudotypes of HIV-1 vector, 4.5×10⁶ 293T packaging cells were co-transfected with 14 μg pHR′CMV-Luc, 14 μg pCMVΔR8.2, and 2 μg DNA plasmid encoding codon-optimized H5HA (see above) with or without various indicated amounts of DNA plasmids encoding codon-optimized NA and M2 using a calcium phosphate precipitation method. As a control 293T cells were also co-transfected with HIV-1-luciferase transfer vector and DNA plasmid encoding VSV-G. After overnight incubation, cells were washed once with HBSS and cultured in 10 ml of complete DMEM supplemented with 100 μM sodium butyrate. 8 hrs later, cells were washed once with HBSS and cultured in 10 ml of complete DMEM. The pseudotype-containing supernatants were harvested in 16 to 20 hrs and the amount HIV-1 gag p24 in the supernatants and/or in the cell lysates of 293FT packaging cells was measured by ELISA as described before (39).

The optimal ratio of the amount of CMVR-HA and CMVR-NA plasmids used for co-transfection was from 8 to 1 to 4 to 1 (FIG. 18). Therefore, in the subsequent HA and NA pseudotype production, 4.5×10⁶ 293T packaging cells were co-transfected with 14 μg pHR′CMV-Luc and 14 μg pCMVΔR8.2, 2 μg CMV/R-HA and 0.5 μg CMV/R-NA using a calcium phosphate precipitation method. As a control 293T cells were also co-transfected with 14 μg pHR′CMV-Luc, 14 μg pCMVΔR8.2, and 5 μg DNA plasmid encoding VSV-G or CCR5-tropism HIV-1 envelope Ad8. After overnight incubation, cells were washed once with PBS and cultured in 10 ml of complete DMEM supplemented with 100 μM sodium butyrate (Sigma, St. Louis, Mo.) for 8 hrs. Cells were then cultured in 10 ml of complete DMEM. The pseudotype-containing supernatants were harvested in 16 to 20 hrs and stored at a −80° C. freezer in aliquots until used in transduction or in neutralization assay (see below).

To test the effect of exogenous NA treatment on H5HA pseudotype release, 293T cells were co-transfected with pHR′CMV-Luc, pCMVΔR8.2 and CMV/R-HA (HA alone) as described above. After overnight incubation, cells were cultured in 10 ml of complete DMEM supplemented with 100 μM sodium butyrate for 8 hrs. Cells were then cultured in 10 ml of complete DMEM in the presence of 0.025 U/ml of Vibrio Cholerae NA (Sigma) as described by Dong et al. (69). The pseudotype-containing supernatants were then harvested in 16 to 20 hrs and cellular debris was pelleted by centrifugation at 2,000×g for 10 minutes.

To characterize pseudotypes, the above collected supernatants were loaded onto 20% sucrose cushion and ultra-centrifuged at 20,000 rpm for 2.5 hours at 4° C. in a Beckman SW41 or SW28 swing rotor (Beckman Coulter, Fullerton, Calif.) dependent on the volumes of supernatants collected. The pellets were dissolved in PBS and further fractionated through a 25-65% sucrose density gradient at 25,000 rpm for 16 hours at 4° C. in a Beckman SW41 swing rotor. Twelve fractions (0.9 ml each) were collected from the top to the bottom of the gradient, TCA precipitated and separated by SDS-PAGE followed by western blot analysis (see below).

Transduction of pseudotypes. In a single-cycle assay to measure the transduction efficiency of pseudotypes, 1×10⁵ MDCK (ATCC CCL-34), CEMss (a human CD4 T cell line provided by Dr. Jon Allan from the Southwest Foundation for Biomedical Research, san Antonio, Tex.), CHO (ATCC CRL-11398), 293T (ATCC CRL-1573), HeLa (ATCC CCL-2), Vero (ATCC CCL-81), HT-29 (ATcc HTB-38) and Caco2 cells (ATCC HTB-37) were transduced with various amounts of pseudotype-containing supernatants in the presence of 1 μg/ml polybrene overnight. Cells were then washed twice with HBSS and cultured in complete DMEM medium for 2 days. Cells were then harvested and washed once with HBSS (without phenol red) and resuspended in 200 μl of HBSS (without phenol red). Luciferase activity in 50 μl of cell suspensions was measured by a BrightGlo Luciferase assay according to the manufacturer's instruction (Promega).

Alternatively, MDCK or Maji-CCR5 cells were transduced with various amounts of pseudotype-containing supernatants in the presence of 1 μg/ml polybrene overnight. Cells were then washed once with PBS and cultured in complete DMEM medium for 2 days. Cells were then washed once with PBS (without phenol red) and suspended in 100 μl of lysis buffer. After single round freeze-thaw, luciferase activity in 30 μl of cell lysates was measured by a BrightGlo Luciferase assay according to the manufacturer's instruction (Promega, Madison, Wis.).

Immunization of mice with plasmid DNA encoding H5HA. Female BALB/c mice at age of 6 week (5 mice per group) were injected (i.m.) with 100 ug of CMV/R plasmid DNA expression vector, CMV/R-HA, CMV/R-HA-mutant-1, or CMV/R-HA-mutant-2, respectively, for three times, at 3-week intervals. Pre-immune and post-immune sera were taken at 7 days before the first immunization and 2 weeks after the third immunization, respectively. Anti-H5HA antibody responses were determined by ELISA and neutralizing assay (see below).

FACS analysis. To study cell surface expression of H5HA, 1×10⁶ mock, H5HA, H5HA/NA, H5/M2, and H5HA/NA/M2-transduced 293 T cells were incubated with the serum from H5HA plasmid DNA immunized mouse (see above) for 40 min on ice. Cells then were washed twice with FACS buffer (PBS containing 1% BSA and 0.02% NaN₃) and further incubated with FITC-conjugated goat anti-mouse IgG Ab for 40 min on ice. Cells then were washed twice with FACS buffer and fixed with 1% formaldehyde in 0.5 ml of FACS buffer. FACS analysis was performed on a FACScan (Becton Dickinson, Mountain View, Calif.).

Neutralizing assay. To determine whether the transduction of H5HA/NA/M2 pseudotypes could be neutralized by sera derived from H5HA-immunized mice. 100 μl of the above produced H5HA/NA/M2 and VSV-G pseudotypes were incubated with or without serial 5 fold dilutions of heat-inactivated pre- and post-immune serum samples for 1 hour at 37° C. The mixtures were then added onto MDCK cells in 24 well plates. After overnight incubation, virus containing supernatants were removed and replaced with fresh complete medium. Transduction efficiency was determined at 48 hours post-transduction by measuring the amount of luciferase activity in transduced cells as described above. Neutralizing activity is displayed as the percentage inhibition of transduction (luciferase activity) at each post-immune serum sample dilution compared with pre-immune serum sample: % inhibition={1−[luciferase in post-immune serum sample/luciferase in pre-immune serum sample]}×100. Titers were calculated as the reciprocal of the serum dilution conferring 50 or 90% inhibition (IC50 or IC90).

Pharmacological inhibition of H5HA/NA/M2 pseudotype entry. To determine whether H5HA/NA/M2 pseudotypes enter cells through the receptor-mediated endocytosis, lysosomotropic agent ammonium chloride (NH₄Cl) and vacuolar H⁺-ATPase inhibitor bafilomycin A1 (BafA1) (Sigma, St. Louis, Mo.) were used to treat cell targets before and during transduction of H5HA/NA/M2 pseudotypes. Working solutions of BafA1 was prepared in dimethyl sulfoxide (DMSO) and stored at −20° C. Stocking solutions of NH₄Cl was prepared in distilled water and sterilized through 0.22 um filter.

To assess the effect of BafA1 and NH₄Cl on H5HA/NA/M2 pseudotype entry, 2×10⁴ MDCK cells per well were seeded onto 24-well plates and pretreated with or without various indicated amounts of BafA1 and NH₄Cl for 1 hour before the transduction. During the transduction, 100 ul H5HA/NA/M2 pseudotype-containing supernatants were added to each well and incubated at 37° C. overnight in the presence of the same drug. The supernatants were then removed and replaced with fresh complete medium. 48 hours after the transduction, cells were harvested and the luciferase activity in transduced cells was measured as described above.

To test the effect of a NA inhibitor on pseudotype release, packaging cells were co-transfected with pHR′CMV-Luc, pCMVΔR8.2, CMV/R-HA and CMV/R-NA or with pHR′CMV-Luc, pCMVΔR8.2 and CCR5-tropism HIV-1 envelope Ad8 as described above. After overnight incubation, cells were cultured in 10 ml of complete DMEM supplemented with 100 μM sodium butyrate for 8 hrs. Cells were then cultured overnight in 10 ml of complete DMEM in the presence of various indicated doses of a NA inhibitor oseltamivir phosphate (Roche Diagnostics). Culture supernatants were collected and used to transduce target cells Maji-CCR5 overnight as described above. The supernatants were then removed and replaced with fresh complete medium. 48 hours after the transduction, cells were harvested and the luciferase activity in transduced cells was measured as described above.

Construction of chimeric H151 HA and H515 HA. Chimeric H151 HA and H515 HA were made by domain swapping between two HA proteins and in particular between two conserved cysteine residues at positions 72 and 294 of H1HA (WSN) SEQ ID NO: 3. To make the chimeric molecule H151 HA open reading frame, in a first and second PCR reaction the first and third domains were isolated from a plasmid containing the H1HA (WSN) open reading frame, SEQ ID NO: 16. In a third reaction the second domain was isolated by PCR from a plasmid containing the H5 HA 2004 Thailand open reading frame, SEQ ID NO: 17. In a final reaction containing all the pooled products of the above PCR reactions and using primers which recognize and anneal to the outmost 5′ portion of the first domain and the outmost 3′ portion of the third domain a final reaction was performed and full length PCR products were obtained by gel purification and DNA sequenced to ensure they were the correct product and had the correct DNA sequence. The resulting nucleic acid coding sequences for H151 HA was SEQ ID NO: 14, and was subcloned into a lentiviral vector as described above for further use and study. PCR cycling conditions were standard for the enzyme and template used and primers were designed using standard techniques.

To make the chimeric molecule H515 HA open reading frame, in a first and second PCR reaction the first and third domains were isolated from a plasmid containing the H5HA Thailand open reading frame, SEQ ID NO: 17. In a third reaction the second domain was isolated by PCR from a plasmid containing the H1HA (WSN) open reading frame, SEQ ID NO: 16. In a final reaction containing all the pooled products of the above PCR reactions and using primers which recognize and anneal to the outmost 5′ portion of the first domain and the outmost 3′ portion of the third domain a final reaction was performed and full length PCR products were obtained by gel purification and DNA sequenced to ensure they were the correct product. The resulting nucleic acid coding sequences for H515 HA was SEQ ID NO:15 and was subcloned into a lentiviral vector as described above for further use and study.

Western blot analysis. To characterize HA and NA pseudotypes, HA and NA pseudotypes-containing supernatants were harvested, concentrated and fractionated in sucrose density gradient as described above. Proteins in concentrated supernatants and fractionated samples were resolved on 12% SDS-PAGE and transferred onto PDVF membranes. Blots were blocked in a solution of Tris-buffered saline containing 5% nonfat dry milk and 0.1% Tween 20 and subsequently probed with a monoclonal antibody (clone 183-H12) specific for HIV-1 gag p24, mouse anti-flag tag monoclonal antibody (Sigma) and mouse immune sera specific for H5HA (see below). Antigens were visualized with an AP-conjugated anti-mouse IgG antibody (Promega) according to manufacturer's instruction.

Electron microscopy. To characterize HA and NA pseudotypes by electron microscopy, 4.5×106 293T packaging cells were co-transfected with pHR′CMV-Luc, pCMVΔR8.2, CMV/R-HA and CMV/R-NA as described above. After the transfection, cells were washed three times with PBS, fixed with 2.5% glutaraldehyde in PBS for 30 minutes at room temperature (RT) and postfixed with 1% osmium tetroxide. The fixed cells were dehydrated with increasing concentrations of ethanol from 50 to 100% and embedded in an epoxy resin mixture. Polymerization was done at 60° C. for 72 hours. The ultrathin sections were stained with uranyl acetate. The sections were then viewed and digitally acquired by a transmission electron microscope (model JEM 1230, JEOL Ltd, Japan)

Production and characterization of lentivirus like particle (LVLP). To generate LVLP, 4×106 293T packaging cells were co-transfected with 14 μg pCMVΔR8.2, 2 μg CMV/R-HA and 0.5 μg CMV/R-NA using a calcium phosphate precipitation method. Two LVLP expressing different H5HA and identical flag-tagged N1NA (A/Thailand/1(KAN-1)/04) on their surface were generated. One LVLP expresses clade 1 HA (A/Thailand/1 (KAN-1)/04) and the other LVLP expresses subclade 2.3 HA (A/Shenzhen/406H/06). After overnight incubation, cells were washed once with PBS and cultured in 10 ml of complete DMEM supplemented with 100 μM sodium butyrate for 8 hrs. Cells were then cultured in 10 ml of complete DMEM. The LVLP-containing supernatants were harvested in 16 to 20 hrs, loaded onto 20% sucrose cushion and ultra-centrifuged at 20,000 rpm for 2.5 hours at 4° C. in a Beckman SW28 rotor (Beckman Coulter, Fullerton, Calif.). The pellets were resuspended in PBS and stored at a −80° C. freezer in aliquots until being used as immunogens (see below).

Immune sera from mice. Female BALB/c mice (Mus musculus) at the age of 6 to 8 weeks were purchased from the Shanghai Institutes of Biological Sciences Animal Center, Shanghai, China, housed in microisolator units and allowed free access to food and water. For immunization, mice were randomly divided into four groups (5 mice per group). The first group was primed and boosted i. m. with DNA plasmid expressing clade 1H5HA (A/Thailand/1(KAN-1)/04). The second group was primed and boosted i. m. with DNA plasmid expressing clade 2.3H5HA (A/Shenzhen/406H/06). The third group was primed i. m. with DNA plasmid expressing clade 1H5HA (A/Thailand/1(KAN-1)/04) and boosted i. m. with LVLP expressing clade 1H5HA (A/Thailand/1(KAN-1)/04) and N1NA (A/Thailand/1(KAN-1)/04). And the last group was primed i. m. with DNA plasmid expressing clade 2.3H5HA (A/Shenzhen/406H/06) and boosted i. m. with LVLP expressing clade 2.3H5HA (A/Shenzhen/406H/06) and the same N1NA. The priming and boosting were performed at a 3 week's interval. Seven days before the immunization and 7 days post immunization, blood was collected via the retro-orbital sinus. After clotting at room temperature for 6 hours and then at 4° C. overnight, tubes were centrifuged and sera were removed, heat-inactivated at 56° C. for 30 minutes and aliquots of pooled immune sera were frozen at −80° C. freezer until used in neutralization assay (see below). All procedures were in accordance with the Chinese Department of Agriculture guidelines for the Care and Use of Laboratory Animals, the Animal Welfare Act and Chinese Department of Agriculture Biosafety guidelines in Microbiological and Biomedical Laboratory.

Immunization and H5N1 challenge in ferrets. Female ferrets (Marshell Farms, North Rose, N.Y.) at the age of 8 to 10 months and serologically negative by HI assay for currently circulating human influenza A or B viruses were used. For immunization, ferrets were randomly divided into 4 groups plus controls. The first group was immunized i. m. with monovalent split-virion inactivated influenza A/Vietnam/1194/2004/NIBRG-14 equivalent to 3.75 μg of HA. The second group was immunized i. m. with the same immunogen equivalent to 30 μg of HA. The third group was immunized i. m. with the same immunogen equivalent to 3.75 μg of HA plus oil-in-water emulsion adjuvant AF03. The fourth group was immunized i. m. with the same immunogen equivalent to 30 μg of HA plus adjuvant alum. As controls, ferrets were injected with saline with or without oil-in-water emulsion adjuvant AF03 or Alum. The immunization was repeated once four weeks later. Two months after the second immunization, the ferrets were intranasally challenged with 20 LD50 of A/Vietnam/1203/04 wild type H5N1 strain. Seven days before the first immunization, 28 days after the second immunization and 21 days post challenge, blood was collected via venipuncture of anterior vena cava. After clotting at room temperature for 6 hours and then at 4° C. overnight, tubes were centrifuged and sera were removed, heat-inactivated at 56° C. for 30 minutes and frozen at −80° C. freezer until used in neutralization assay (see below).

HA and NA pseudotype-based assay. MDCK cells (2×104 cells per well) were seeded onto 24 well culture plate in complete DMEM overnight. To test the neutralization activity of immunized mouse and ferret sera, the serum samples were serially diluted and incubated with indicated amounts of pseudotypes at the final volume of 50 μl at 37° C. for 1 hour. The mixture was added to cultures of MDCK cells. After the overnight incubation, cells were then washed with phosphate buffered saline (PBS) and cultured in complete DMEM medium for 48 hours. Cells were then detached by trypsin-EDTA treatment and luciferase activity (RLA) was measured as described above. The % inhibition was calculated by (RLA in pseudotypes and medium control—RLA in pseudotypes and immune serum in a given dilution)/RLA in pseudotypes and medium control. The 50% Inhibitory concentration (IC50) and IC95 were determined as the dilutions of a given immune serum that result in 50 and 95% reduction of luciferase activity, respectively.

HI assay. Viruses A/Shenzhen/406H/06 and A/Vietnam/1203/04 were diluted to 8 HA units and incubated with an equal volume of serially diluted immune sera and sera from infected ferrets for 30 minutes at room temperature. An equal volume of 0.5% chicken or horse red blood cells was added to the wells and incubation continued on a gently rocking plate for 30 minutes at room temperature. Button formation was scored as evidence of hemagglutination inhibition (HI).

Micro-neutralization assay. MDCK cells (5×103 cells per well) were seeded onto a 96 well culture plate in complete DMEM overnight. To test neutralization activity of immune sera, serially 2-fold diluted sera (starting at 1:10 dilution) were incubated with 100TCID50 wild type viruses A/Shenzhen/406H/06 and A/Vietnam/1203/04 at the final volume of 50 μl at 37° C. for 1 hour. After the incubation, the mixture was added onto MDCK cells. The CPE (Cytopathic effect) was scored at 4 days after infection. CPE was compared to the positive control (virus-inoculated cells) and negative control (mock-inoculated cells). The assay was performed in triplicate.

### EXAMPLE 2

### Co-Transfection of NA, but not M2 Alone, Enhances Cell Surface Expression of H5HA in Packaging Cells

To determine the effect of co-transfection of DNA plasmid encoding NA or M2 on cell surface expression of H5HA, 293 T packaging cells were transfected with a lentiviral transfer vector pHR′CMV-Luc and a packaging vector pCMVΔR8.2 and DNA plasmid encoding H5HA with or without various amounts of DNA plasmids encoding NA or M2 (FIG. 1A). At 48 hours post transduction, 293 T packaging cells were stained with anti-H5HA-specific immune serum.

In contrast to the previous report on FPV H7HA (11), without co-transfection of NA and/or M2, H5HA alone does express on the cell surface of packaging cells (FIG. 1A) and co-transfection of H5HA and M2 does not enhance the cell surface expression of H5HA (FIG. 1B).

Co-transfection of H5HA and NA or co-transfection of H5HA, NA and M2 enhances the cell surface expression of H5HA. However, the level of H5HA expression in cells co-transfected with H5HA and NA and in cells co-transfected with H5HA, NA and M2 is very similar, indicating that it is NA, but not M2, that enhances cell surface expression of H5HA in packaging cells (FIGS. 1C and 1D).

In addition, it was found that supernatants harvested from cells co-transfected with H5HA and NA and from cells co-transfected with H5HA, NA and M2 contain a similar amount of p24, which is about 2-fold higher than supernatants harvested from cells transfected with H5HA alone.

### EXAMPLE 3

### Co-Transfection of NA, but not M2 Alone, Significantly Enhances Transduction Efficiency of H5HA-Pseudotyped Lentiviral Vectors

To determine the effect of co-transfection of DNA plasmid encoding NA or M2 on transduction efficiency of lentiviral vector, 293FT packaging cells were transfected with HIV-1-luciferase transfer vector and DNA plasmid encoding H5HA with or without various amounts of DNA plasmids encoding NA or M2. Supernatants containing recombinant pseudotypes were harvested and used to transduce MDCK cells with an equal amount of HIV-1 gag p24. Transduction efficiency was measured by relative luciferase activity at 48 hrs post-transduction.

As shown in FIG. 2A without co-transfection of plasmid DNA encoding NA or M2, very low, but measurable, relative luciferase activity (RLA over 1,000) was detected in H5HA alone pseudotyped lentiviral vector. However, co-transfection of plasmid DNA encoding NA with H5HA significantly increased transduction efficiency (RLA ranging from 500,000 to 7,000,000) dependent upon the amounts of plasmid DNA of NA co-transfected.

The best transduction efficiency was obtained when the ratio of HA and NA constructs was at 4:1 or 8:1. In contrast, co-transfection of plasmid DNA encoding various amount of M2 alone with H5HA resulted in no transduction efficiency at all (RLA less than 100) (FIG. 2B). The experiments were repeated five more times with similar results. Thus, these results clearly demonstrated that co-transfection of NA, but not M2, with H5HA significantly (close to 4 log) enhance transduction efficiency of H5HA-pseudotyped lentiviral vector.

### EXAMPLE 4

### Effect of M2 on Transduction Efficiency of H5HA/NA-Pseudotyped Lentiviral Vector

The inventors next investigated the effect of M2 on transduction efficiency of lentiviral vector co-transfected with both H5HA and NA. To accomplish this, 293 T packaging cells were co-transfected with H5HA and NA at the optimal ratio (4:1) with or without various amount of M2. Supernatants containing recombinant pseudotypes were harvested and used to transduce MDCK cells. Transduction efficiency was measured by relative luciferase activity at 48 hrs post-transduction.

FIG. 3 shows the RLAs of MDCK cells transduced with H5HA/NA pseudotypes with or without co-transfecting various amounts of M2. Compared to the cells transduced with lentiviral vector pseudotyped with co-transfection of H5HA and NA (RLA 7,000,000), co-transfection of M2 results in moderate (about 2-3 folds) increase in transduction efficiency. In addition, the inventors also found that at the suboptimal H5HA and NA ratios (1:1.25 or 16:1), co-transfection of M2 results in minimum increase or even decrease of transduction efficiency (data not shown). Thus, it appears that the moderate (2-to-3-fold) increase by M2 is not only M2 dose dependent, but also depends on the proper ratio of H5HA and NA. Furthermore, this increase is much lower than the increase seen in the previously reported lentiviral vector pseudotyped with FPV H7HA, NA, and M2 (11).

### EXAMPLE 5

### Host Range of H5HA/NA/M2-Pseudotyped Lentiviral Vectors

The inventors also compared the transduction efficiency of H5HA/NA/M2- and VSV-G-pseudotyped lentiviral vectors in eight different cell lines CHO, MDCK, 293 T, HeLa, Vero, Caco2, HT29 and CEMss. To accomplish this, cells were transduced with supernatants containing either H5HA/NA/M2- or VSV-G-pseudotyped lentiviral vectors (equivalent to 10 ng of HIV-1 gag p24) in the presence of polybrene. At 48 hours post transduction, luciferase activity in transduced cells was measured as described above.

FIG. 4 shows the relative luciferase activity detected in each of these 8 cell lines. Except for Vero cells, the transduction efficiency in all other cell lines is comparable or higher when transduced by H5HA/NA/M2 pseudotypes than by VSV-G pseudotypes.

### EXAMPLE 6

### Cell Entry of H5HA/NA/M2-Pseudotyped Lentiviral Vector is Through Receptor-Mediated Endocytosis

Next, it was determined whether H5HA/NA/M2-pseudotypes enter cells through receptor mediated endocytosis. To accomplish this, MDCK target cells were pretreated with various doses of bafilomycin A1 or NH₄Cl. Since bafilomycin A1 was dissolved in DMSO, MDCK target cells were also pretreated with equal amount of DMSO as controls. After the pretreatment, cells were transduced with supernatants containing H5HA/NA/M2 pseudotypes. Luciferase activity was measured at 48 hours post transduction.

FIG. 5 shows the relative luciferase activity detected in MDCK cells with or without the pretreatment of bafilomycin A1 or NH₄Cl. In the both pretreatments, RLA was reduced in a dose-dependent manner. Pretreatment of cells with 10 nM bafilomycin A1 resulted in 1 log reduction of transduction efficiency. Pretreatment of cells with 50 and 100 nM bafilomycin A1 resulted in 2 log or more reduction of transduction efficiency (FIG. 5A) Pretreatment of cells with 1 MM NH₄Cl resulted in 50% reduction of transduction efficiency. Pretreatment of cells with 10 Mm NH₄Cl resulted in 1 log reduction of transduction efficiency (FIG. 5B). Thus, the inventors determined that 5HA/NA/M2-pseudotypes enter cells through receptor-mediated endocytosis.

### EXAMPLE 7

### H5HA Specific Immune Sera, but not Pre-Immune Sera, Neutralize Cell Entry of H5HA/NA/M2-Pseudotypes

The inventors determined whether H5HA/NA/M2-pseudotypes can be neutralized by H5HA-specific antibodies. To test this, 100 μl of supernatants containing either H5HA/NA/M or VSV-G-pseudotypes were incubated with various dilutions of pre-immune or post-immune sera specific for H5HA (see the Materials and Methods for the detail) at 37° C. for 1 hour. After the incubation, pseudotypes and sera mixture was added onto MDCK target cells for the transduction. Luciferase activity was measured at 48 hours post transduction. Because VSV-G envelope interacts with lipid moiety in the lipid bilayer of the cytoplasmic membrane, VSV-G pseudotypes bypass the requirement of the interaction between HA envelope and its sialic acid-containing receptors to enter cells. Therefore, they were used as a negative control. Neutralizing activity of post-immune serum samples is displayed as the percentage inhibition of transduction (luciferase activity) at each dilution of post-immune samples compared with pre-immune serum samples: % inhibition={1−[luciferase in post-immune serum sample/luciferase in pre-immune serum sample]}×100. Titers were calculated as the reciprocal of the serum dilution conferring 50 or 90% inhibition (IC50 or IC90).

FIG. 6 shows the percentage of inhibition of pooled post-immune serum samples from mice immunized with plasmid DNA containing a H5HA mutant (see the Materials and the Methods). At the 1 to 250 dilution, the inhibition is almost 100%. At the 1 to 500 dilution, the inhibition is 90%. At the 1 to 1000 dilution, the inhibition is almost 62%. At the 1 to 2000 dilution, the inhibition is 50%. But at the same dilutions no inhibition against VSV-G pseudotypes was detected (FIG. 6).

### EXAMPLE 8

### Further Investigation into the Mechanism of NA Enhancement of H5HA

To determine the mechanism NA enhancement, the inventors compared the amount of HA and HIV-1 gag protein in transfected cells, concentrated supernatants, and pseudoparticles among cells transfected with H5HA alone with or without exogenous NA treatment or co-transfected with H5HA/NA.

It was found that although H5HA possesses a multibasic cleavage site, only 50% HA₀ was cleaved into HA₁ and HA₂ and the amount of HA₀, cleaved HA and HIV-1 gag was similar regardless of cells transfected with H5HA alone or co-transfected with H5HA/NA, see FIG. 8 cell lysis panel.

However, much more cleaved HA and HIV-1 gag and fewer HA₀ was detected in concentrated supernatants of cells co-transfected with H5HA/NA than transfected with H5HA alone, see FIG. 8, supernatant panel.

Moreover, it was found that much more H5HA was detected in pseudoparticles produced by cells co-transfected with H5HA/NA than by cells transfected with H5HA alone plus exogenous NA treatment, see FIG. 9.

Thus, in H5HA/NA pseudotyped lentiviral vector, co-transfected NA uses two previously unknown mechanisms to enhance transduction efficiency of pseudotypes, i.e. preferential release of pseudotypes with cleaved H5HA and increased incorporation of the amount of H5HA into pseudoparticles.

### EXAMPLE 9

### Novel Anti-HA Antibody Neutralizing Activity Assay

Currently the standard assays to measure neutralizing activity of anti-HA antibodies, sera from immune individuals and also sera from infected individuals, are 1) hemagglutinin inhibition (HI) assay and 2) micro-neutralization assay.

The HI assay is a surrogate assay, which may not reflect real neutralizing activity of antibodies. This test makes use of the principle that hemagglutinin agglutinates erythrocytes (red blood cells, RBCs) to identify the virus. When antibodies raised against the hemagglutinin antigen are added, they will bind to the antigenic sites in the HA molecule and inhibits the binding between the HA and the receptors on the RBCs. Thus, when there is presence of HA, antibodies will bind to the HA and prevent hemagglutination of the RBCs.

The microneutralizing assay uses wild type virus and a cell-based assay that more accurately measures the neutralizing activity of anti-HA antibodies.

However, the read-out of this assay is a cytopathic effect (CPE) which is observed under the microscope. Therefore, it is a subjective measurement and so hard to quantify and develop into a reproducible system between samples and users. In addition, because the assay uses wild type virus for infection, it can only be performed safely in a biocontainment level 3 or higher laboratory if a high pathogenic influenza virus, such as H5N1, is used.

Therefore, the inventors have based upon the work described in this patent application set out to develop a new H5HA/NA pseudotyped lentiviral vector-based neutralizing assay that, it is believed, will eventually replace the current assays used to measure neutralizing activity of anti-HA antibodies, sera from immune individuals and also infected sera; because this H5HA/NA pseudotyped lentiviral vector-based neutralizing assay provides objective and quantified data. It requires only biocontainment level 2 because of use of pseudotypes, instead of wild type viruses.

To accomplish this, the inventors have generated a panel of H5HA/NA pseudotypes that covers major subclades of H5HA, see FIG. 10. Using mouse immune sera specific against an H5HA (subclade 1.1) and convalescent human sera for H5N1 (subclade 2.3) infected human individual, the inventors carried out parallel neutralization studies.

The inventors compared neutralization titers of HI assay see FIG. 11A, microneutralization assay see FIG. 11B and the new H5HA/NA pseudotype-based neutralization assay, see FIG. 12.

The results of this comparison demonstrated that not only does the pseudotype-based neutralization assay give parallel results to the microneutralization assay, it is also far more sensitive (at least two log more) and quantifiable.

More importantly, the inventors found that although there is certain degree of cross-reactivity between subclades 1.1 and 2.3, neutralization titers against homologous subclades are much higher than heterologous subclades, emphasizing the importance of using a panel of pseudotypes covering all major subclades of H5HA for any serological survey of neutralization activity of vaccinated or infected individuals (see FIGS. 10 to 12).

Thus, the inventors have clearly demonstrated that this new H5HA/NA pseudotyped lentiviral vector-based neutralizing assay is much more sensitive and quantifiable than the microneutralizing assay. In addition due to the materials used in this new assay, it can be safely performed in a larger range of labs by less experienced individuals.

### EXAMPLE 10

### Use of H5HA/NA Pseudotyped Lentivirus in a New Screening Method for Anti-Viral Compounds

The inventors have also used the H5HA/NA pseudotyped lentiviral vector, to also develop a cell-based assay to screen anti-virals.

Using this assay to screen small compound library isolated from some traditional Chinese medicine by HPLC, two compounds (1 and 2) with EC50 at about 5 μM (FIGS. 13A and 13C) and SI>25 and 9.12, respectively were identified (see FIG. 13). CC50 is the compound dose which results in 50% cytotoxicity; EC50 is the compound dose which results in 50% inhibition of viral transduction or infection. SI, or safety index was calculated as follows: SI=CC50/EC50. Usually, a compound with a high SI is safer (less cytotoxicity). Equally importantly, during the assay development the inventors also successfully streamlined steps of the assay in a 96 well format by combining transduction, cell culture and measurement of luciferase activity in a single well.

### EXAMPLE 11

### Additional Viral Pseudotypes

The inventors have continued to expand the panel of HA/NA pseudotypes, besides major H5HA sub-clades mentioned above, and have also generated HA/NA pseudotypes expressing H1HA, H2HA, and H7HA. In addition, they have also made chimeric HA between H1HA and H5HA (Table I) and proven they have biologic activity and highly immunogenic.

These new pseudotypes expressing chimeric HA will be very useful tools to dissect antigenicity and immunogenicity of HA molecules, which, in turn, will lead to develop new and better vaccine candidates.

TABLE I

Control

| | 63 | 71 |
|---|---|---|
| H1/N1 | 1064645 | 1237576 |
| H5/N1 (Th) | 2911097 | 3045658 |
| H151/N1 (Th) | 1577439 | 1765842 |
| H515/N1 (Th) | 1653841 | 1762159 |

In Table I, Th relates to proteins derived from A/Thailand/(KAN-1)/2004H5N1 avian flu strain.

Table I shows relative luciferase activity of MDCK cells transduced with pseudotypes expressing H1HA/N1NA, H5HA/N1NA, chimeric H151HA/N1NA or chimeric H515HA/N1NA. Chimeric peptide H151HA (SEQ ID NO: 12) and chimeric peptide H515HA (SEQ ID NO: 13) were constructed by domain swapping between H1HA (SEQ ID NO: 3) and H5HA (SEQ ID NO: 4) between the conserved cysteine residues located at positions 72 and 294 in SEQ ID NO: 3. Hence H151HA (SEQ ID NO: 12) comprises residues 1-72 from H1HA (SEQ ID NO: 3), residues 72-293 from H5HA (SEQ ID NO: 4) and residues 295-569 of H1HA (SEQ ID NO: 3). Likewise H515HA (SEQ ID NO: 13) comprises residues 1-71 from H5HA (SEQ ID NO: 4), residues 73-294 from H1HA (SEQ ID NO: 3) and residues 294-568 of H5HA (SEQ ID NO: 4). In the same way other combinations of portions of two or more HA protein domains could be created by swapping domains between conserved residues. With reference to the alignments of the HA and NA proteins enclosed herein as FIGS. 14 and 15, several conserved residues ranged throughout these two proteins are shown. This together with the complete peptide sequences of these various HA and NA proteins provides the basis for the creation of various combinations of domains from these proteins into new chimeric forms.

### EXAMPLE 12

### Use of Pseudotyped Lentivirus Particles in Pseudotyping Assay for Neutralizing Serological Antibodies

Production and Characterization of H5HA and N1NA Pseudotypes

Production of H5HA and N1NA expressing pseudotypes derived from H5N1 strains was conducted using the 293T cell line. These cells were cotransfected with the transfer vector pHR′CMV-Luc, the packaging vectors pCMVRΔ8.2, CMVR-H5HA and CMVR-N1NA. After transfection, culture supernatants containing H5HA and N1NA pseudotypes were harvested, concentrated and then fractionated through a sucrose gradient.

The H5HA, N1NA and HIV-1 gag proteins in each fraction were detected with specific antibodies (see the Materials and Methods above for details). FIG. 16a shows that the H5HA and N1NA proteins co-migrated with HIV-1 gag protein in the sucrose gradient indicating that both H5HA and N1NA were incorporated into the pseudotype particles. FIG. 16b is an electronmicrograph showing H5HA and N1NA pseudotypes being formed and released from the surface of the 293 T packaging cells.

FIG. 16c shows the transduction efficiency measured by relative luciferase activity (RLA) in transduced MDCK target cells. As expected, the RLA in cells transduced with pseudotype particles contained in supernatants from cells transfected with pHR′CMV-Luc and pCMVRΔ8.2 alone was similar to mock transduction. In contrast, low but measurable RLA was detected in cells transduced with pseudotype-containing supernatants from cells transfected with pHR′CMV-Luc and pCMVRΔ8.2 plus CMVR-H5HA alone. In contrast, in cells transduced with supernatants from cells transfected with pHR′CMV-Luc, pCMVRΔ8.2 and CMVR-H5HA with the addition of exogenous NA, over 1,000-fold higher RLA was detected. Surprisingly, in cells transduced with supernatants from cells co-transfected with pHR′CMV-Luc and pCMVRΔ8.2 plus CMVR-H5HA and CMVR-N1NA five logs higher RLA was seen than with H5HA expression alone and two logs higher than H5HA expression combined with exogenous NA treatment. This experiment was repeated three times with similar results.

Cellular Entry and Release of H5HA and N1NA Pseudotypes

To determine if H5HA and N1NA pseudotypes enter cells through receptor mediated endocytosis, Maji-CCR5 target cells were pretreated with bafilomycin A1 or NH₄Cl. Both agents inhibit acidification of the endosomes and block endocytosed virus from entering the cytosol. After the bafilomycin A1 or NH₄Cl pretreatment, cells were transduced with supernatants containing H5HA and N1NA pseudotypes or pseudotypes expressing CCR5-tropic HIV-1 envelope Ad8. The latter is known to enter the cell directly by passing through plasma membrane (58). FIGS. 16d and 16e show RLA in cells with or without the pretreatment of NH₄Cl and bafilomycin A1, respectively.

Either pretreatment resulted in significant reduction in transduction efficiency of pseudotype particles expressing H5HA and N1NA but did not affect the transduction efficiency of pseudotypes expressing HIV-1 envelope. Significant reduction of transduction efficiency of H5HA and N1NA pseudotypes was also observed in MDCK target cells using the same pretreatment (data not shown). The results of these studies suggest that pseudotypes expressing H5HA and N1NA enter cells through receptor-mediated endocytosis.

To determine if sialidase (also called Neuraminidase) activity of NA is required for H5HA and N1NA pseudotype release and entry, 293T cells were transfected with pHR′CMV-Luc and pCMVRΔ8.2 plus CMVR-H5HA and CMVR-N1NA, with pHR′CMV-Luc and pCMVRΔ8.2 plus VSV-G or with pHR′CMV-Luc and pCMVRΔ8.2 plus HIV-1 envelope Ad8. After the transfection, the cells were treated with or without various doses (from 4 to 500 μM) of the neuraminidase inhibitor oseltamivir phosphate. Culture supernatants were collected and used to transduce the Maji-CCR5 target cells.

FIG. 16f shows that treating 293 T cells with oseltamivir phosphate induced a marked dose-dependent decrease in the transduction efficiency of pseudotypes expressing H5HA and N1NA, in comparison to pseudotypes expressing HIV-1 envelope or VSV-G. In contrast, treatment of Maji-CCR5 target cells with the same amount of oseltamivir phosphate did not reduce transduction efficiency of either the H5HA and N1NA pseudotypes or the HIV-1 envelope pseudotypes or VSV-G pseudotypes (FIG. 16g). Taken together the results of the pseudotype characterization studies suggest that the mechanisms of cellular entry and release of pseudotypes expressing H5HA and N1NA are similar to those of wild type influenza A virus.

Generation of a HA and NA Pseudotype Panel

The inventors next generated a pseudotype panel containing pseudotype particles that expressed eight different H5 HAs with the same N1NA (A/Thailand/1(KAN-1)/04). Pseudotypes co-expressing a H1HA cleavage mutant of WSN with A/Thailand/1(KAN-1)/04 N1NA and pseudotypes expressing VSV-G were also generated for use as controls. All eight H5HAs were derived from H5N1 virus strains isolated from human infections. Table II shows the HA/NA pseudotype panel generated for use in this study.

From the prototype HA and NA pseudotype, the inventors have developed a pseudotype panel including all clades and subclades of H5HA as well as H1HA, H2HA H7HA and H9HA. In H1HA, the inventors made pseudotypes expressing the new swine H1HA

After the HA/NA pseudotype panel was generated, the inventors measured the titers of the pseudotypes and evaluated the correlation between the doses of pseudotype particles and RLA. The titers of the two HA/NA pseudotypes were 5.01×10⁷ and 5.76×10⁶ TU (transducing units)/ml, respectively. For both pseudotypes tested, the inventors observed near perfect correlation between the TU ranging from 1×10² to 5×10⁵ and the RLA ranging from 1,000 and 5,000,000 (FIGS. 18b and 18d). In view of these results, the inventors used pseudotype doses corresponding to 40,000, 200,000 and 1,000,000 RLA units in subsequent PN assay development.

Development of the HA and NA Pseudotype-Based Neutralization (PN) Assay.

To develop the PN assay, the inventors used pooled sera from mice immunized with one of two immunization protocols: 1) pDNA/pDNA prime-boost, and 2) DNA prime and VLP boost (see Materials and Methods for details). The specificity and the cross-reactivity of the immune mouse sera were tested against three different HA and NA pseudotypes and control pseudotypes that expressed VSV-G. The three HA and NA pseudotypes contained a common N1NA (A/Thailand/1(KAN-1)/04) and one of two H5 hemagglutinins, (A/Thailand/1(KAN-1)/04; clade 1 or A/Shenzhen/406H/06, subclade 2.3).

H1HA pseudotypes were produced using a H1HA cleavage mutant from the H1N1 strain WSN. As shown in FIG. 17, immune sera elicited by priming and boosting with plasmid DNA expressing H5HA from H5N1 subclade 2.3 effectively neutralized pseudotypes expressing subclade 2.3H5HA. The same sera weakly neutralized clade 1H5HA and did not neutralize H1HA/N1NA or VSV-G pseudotypes (FIG. 17a). Sera from mice immunized with pDNA encoding H5HA from subclade 2.3 followed by boosting with lentivirus-like particles (LVLP expressing both H5HA from subclade 2.3 and N1NA showed similar results. (FIG. 17c).

Likewise, sera from mice primed and boosted with pDNA encoding H5HA from clade 1 most effectively neutralized pseudotypes expressing clade 1H5HA. The same sera weakly neutralized subclade 2.3H5HA and did not neutralize pseudotypes expressing H1HA/N1NA or VSV-G (FIG. 17b). Prime-boost studies using H5N1 clade 1 pDNA followed by H5N1 clade 1 LVLPs showed similar results. (FIG. 17d). Thus, the results from these studies demonstrated that the PN assay exhibits good specificity and reveals quantitative differences in neutralization activity of immune sera elicited with H5N1 clades and subclades.

Comparison of the PN Assay with the MN Assay

The inventors next used sera of vaccinated mice to compare the sensitivity of their PN assay with a MN assay. In these studies, mice were immunized with pDNA or pDNA and LVLPs expressing H5HA (A/Shenzhen/406H/06) and N1NA (A/Thailand/1(KAN-1)/04).

In the PN assay the immune sera were titrated against pseudotypes expressing H5HA (A/Shenzhen/406H/06, subclade 2.3) while in the MN assay the same immune sera were titrated against wild type H5N1 virus (A/Shenzhen/406H/06). In the MN assay wild type virus was used at 100 TCID₅₀ as described by Rowe et al. (43) and the neutralization titer of serial dilutions of immune sera was obtained by assessing CPE in MDCK cells infected with H5N1 virus (A/Shenzhen/406H/06). The CPE scores were based on the morphology of MDCK cell monolayer observed microscopically (FIG. 19). In the PN assay, the neutralizing titer of serial dilutions of immune sera was obtained by determining percent inhibition of transduction efficiency in MDCK cells transduced with H5HA/N1NA pseudotypes (A/Shenzhen/406H/06). To determine the effect of input doses of pseudotype on the measurement of neutralization titers by the PN assay the inventors titrated immune sera against three doses of HA and NA pseudotypes corresponding to RLA values of 1,000,000, 200,000 and 40,000 (FIG. 20).

As shown in Table III, there is an excellent correlation between the neutralization titers measured by the MN assay and PN assay when 100 TCID₅₀ was used for the MN assay and RLA 1,000,000 for the PN assay.

For example, immune sera elicited by a pDNA/pDNA prime/boost protocol, complete inhibition of CPE was found at dilutions of 1/10 and 1/20; while CPEs ranging from weakest (+) to strongest (++++) were scored at dilutions from 1/40 to 1/640. Similarly in the PN assay, at the input dose of RLA 1,000,000 the same sera showed 99.9% inhibition at dilutions of 1/10 and 1/20 and 94.6 to 21.7% inhibition at dilutions from 1/40 to 1/640. Moreover, the PN assay is somewhat more sensitive than MN assay. For example at the 1:640 dilution, immune sera elicited with the pDNA/pDNA prime/boost protocol exhibited no inhibition measured by the MN assay while measured by the PN assay the same sera at dilutions of 1:640 and 1:1,280 dilutions still showed 21.7 and 11.3% inhibition, respectively.

Finally, lowering the input dose of HA/NA pseudotypes significantly increased the neutralization activity measured by the PN assay. For example, at RLA 1,000,000 immune sera elicited with pDNA/pDNA prime/boost protocol showed 11.3% inhibition at 1:280 dilution, whereas at RLA 40,000 the same sera at the same dilution exhibits 51.2% inhibition.

Evaluation of the PN Assay in an Influenza Vaccine Model

To evaluate the usefulness of the PN assay, the inventors used it to measure neutralizing antibody responses after vaccination with human candidate H5N1 vaccines in the ferret, an established clinically relevant model for human influenza. In these studies the inventors compared neutralization titers of sera from ferrets immunized with a monovalent split-virion inactivated H5N1 (A/Vietnam/1194/2004/NIBRG-14) vaccine at a dose of 3.75 μg and 30 μg HA with or without the adjuvant. Adjuvants used were an oil-in-water emulsion-based adjuvant (AF03; sanofi pasteur) or aluminum hydroxide adjuvant (A100H). After immunization the ferrets were challenged with wild type H5N1 (A/Vietnam/1203/04) virus measured by PNA, MNA and HIA. All ferrets in the experiment survived after the challenge except for PBS control (#6360), alum control (#6369) and two adjuvant AF03 controls (#6286 and #6303) (data not shown).

Table IV shows that neutralization titers measured by these three assays correlate very well. Moreover, the PN assay also appears more sensitive than the MN and HIA assays for detecting neutralizing antibody responses to influenza viruses. For example, two of immune sera (#6283 and #6320) elicited with split-virion equivalent to 3.75 μg HA alone and one of immune sera (#6278) elicited with split-virion equivalent to 30 μg HA alone exhibited undetected neutralization titers when measured by both MNA and HIA. In contrast, when measured by PNA low, but measurable, neutralization titers were detected. Thus, the inventors conclude that overall there is a good correlation among neutralization titers measured by HI, MN and PN assays. However, in immune sera with very low neutralization activity the PN assay exhibits better sensitivity than HI and MN assays

To evaluate the sensitivity and antigen specificity of the PN assay the inventors next measured neutralization titers of immune and challenged ferret sera against pseudotypes expressing seven different H5 HAs and one H1 HA. All H5 HA pseudotypes also expressed a common N1 NA from the A/Thailand/1(KAN-1)/04H5N1 strain. Table V shows that sera from H5N1 vaccinated ferrets cross neutralized at least one of the heterologous H5HA pseudotypes. In contrast, sera from H5N1 immunized ferrets showed no neutralization activity against pseudotypes expressing H1HA and the A/Thailand/1(KAN-1)/04 NA.

Serum samples from ferrets immunized with the low dose vaccine (3.75 μg HA) (#6283, #6320 and #6398) showed low to undetectable neutralization titers against homologous H5 HA/NA pseudotypes (Table IV). Sera from these animals also had low or undetectable neutralization titers against heterologous pseudotypes expressing H5HA from H5N1 clades 0, 1 or 2.3. Higher neutralization titers were seen against clades 0 and 1 than against subclade 2.3. In contrast, immune sera (#6282, #6297 and #6304) elicited with split-virion vaccine at a dose of 3.75 μg HA with AF03 adjuvant exhibited much higher neutralization titers against homologous H5HA and N1NA pseudotypes (see Table IV). These sera also showed higher neutralization titers against heterologous pseudotypes expressing H5HA from H5N1 clades 0, 1, 2.1 and 2.3. The highest neutralization titers were against pseudotypes expressing H5N1 clade 1 HA with lower titers observed against pseudotypes expressing HA from H5N1 clades 2.1 and 2.3 with the lowest neutralizing activity seen against pseudotypes expressing H5 HA from clade 1. Similarly, Table IV shows that sera from ferrets immunized with A100H-adjuvanted split-virion vaccine at a dose of 30 μg HA exhibited the highest neutralization titers against pseudotypes expressing H5HA and N1NA. These sera also exhibited the highest neutralization titers against heterologous pseudotypes expressing different clade and subclades of H5HA. Neutralization titers were higher against clade 1 than against clades 0, 2.1 and 2.3. After the challenge, not only did all sera from vaccinated ferrets exhibit much higher neutralization titers against all H5HA and N1NA pseudotypes, but also showed detectable neutralization activity against pseudotypes expressing H1HA and N1NA. From the results of these studies, the inventors conclude that ferrets immunized with split-virion vaccines prepared from H5N1 clade 1H5HA developed neutralizing antibody responses against H5HA from different sublineages of clade 1. Sera from immunized ferrets also showed moderate cross-neutralization against H5HA from clades 0, 2.1 and 2.3 but these sera did not cross-neutralize pseudotypes expressing H1HA and N1NA.

Intriguingly, immunized ferrets challenged with wild type H5N1 virus rapidly produced high levels of cross-neutralizing antibody against H5N1 clades 0, 1, 2.1 and 2.3. Post challenge boosting was seen in all immunized ferrets, regardless of the dose of HA or the adjuvant used. However, higher cross-neutralization titers were seen against pseudotypes expressing HA from within the various strains of H5N1 clade 1 viruses than clades 0, 2.1 and 2.3. Also after wild-type H5N1 challenge, sera from H5N1 immunized ferret showed neutralizing antibody responses cross-reactive against pseudotypes expressing H1HA were also detected.

The development of effective immunogens that elicit neutralizing antibody responses against genetically diverse strains of HPAI H5N1 viruses requires both the identification of appropriate HA and NA antigenic structures (39) and the identification of epitopes that induce protective antibodies (40), Moreover, the development of candidate H5N1 pandemic vaccines influenza vaccines requires for standardized in vitro assays that will allow for a meaningful comparison of the potency and the breadth of neutralizing antibody responses in sera or other body fluids from HPAI H5N1 vaccinated subjects.

In this study, the inventors described a method to produce working quantities of H5N1 HA and NA expressing pseudotypes. The inventors prepared a panel of these HA and NA pseudotypes that included the major H5N1 clades and subclades that have been isolated from human infections. We also demonstrated that HA and NA pseudotypes mimic wild type of influenza virus in their mechanisms of cellular entry and release (see FIG. 16). Using immune mouse and ferret sera the inventors developed a pseudotype-based neutralization (PN) assay and showed that this assay exhibited good specificity and can be used to measure quantitative differences in neutralization activity against different clades and subclades of H1N1 HPAI influenza viruses (FIG. 17 and Table V). The inventors also demonstrated excellent correlation between neutralization titers measured by the MN and PN assays. Moreover, the PN assay was found to be somewhat more sensitive assay the MN or HI assay, since it was able to detect low level specific antibody responses that the other two assays could not (Tables III and IV). Finally, the inventors used sera from ferrets immunized with split virion H5N1 vaccines to show the PN assay to be a sensitive and quantifiable assay for measuring neutralizing antibody responses against diverse H5N1 clades and subclades. In these experiments, the inventors compared antibody responses measured using the PN assay to those measured by the MN and HI assays (Table IV and V).

Additionally, these results illustrate the importance of choosing appropriate the pseudotype doses for measuring neutralizing antibody responses against HPAI H5N1 viruses by PN assay (Table III). For use in evaluation of neutralization antibody titers elicited with vaccine candidates higher input doses of HA and NA pseudotypes that can make a meaningful comparison with 100 TCID₅₀ used in standard MNA should be used in PN assay, so that neutralization titers of immune sera or other body fluids measured by PN assay will not be overestimated. In contrast, for use in serodiagnosis, lower input doses of HA and NA pseudotypes may be used, which serves to increase the sensitivity of the assay without sacrificing its specificity.

While these results showed the same requirement of low pH for the entry of HA and NA pseudotypes as pseudotypes expressing H5HA alone described by Nefkens et al. (14), our results also point out major limitations using a H5HA pseudotype expressing a single clade of H5HA in serodiagnosis (14). In this study, the inventors show that pseudotypes co-expressing HA and NA have much higher transduction efficiency than pseudotypes expressing H5HA alone plus exogenous NA, suggesting that co-transfection of lentiviral transfer vector with H5HA alone plus exogenous NA treatment during pseudotype release results in less pseudotype production compared to pseudotypes co-expressing HA and NA (see FIG. 16c). Moreover, the inventors show that there is significant quantitative difference in antigenicity and immunogenicity among different of hemagglutinins from various H5N1 clades and subclades (FIG. 17 and Table V). Thus, pseudotypes expressing H5HA from a single clade may fail to detect neutralization activity in sera from humans and animals infected with H5N1 viruses from other clades or subclades.

TABLE II
The list of HA and NA pseudotypes generated and used in this study

| | Clade | |
|---|---|---|
| | or subclades | Accession |
| Pseudotyped lentiviral vector | of H5HA | Number |
| H1HA A/WSN/33/N1NA* | | J02176 |
| H5HA A/Cambodia/P0322095/05/N1NA | 1 | P0322095** |
| H5HA A/Cambodia/Q0321176/06/N1NA | 1 | Q0321176** |
| H5HA A/Thailand/1 (KAN-1)/04/N1NA | 1 | EF107522 |
| H5HA A/Vietnam/1203/04/N1NA | 1 | EF541403 |
| H5HA A/Indonesia/5/05/N1NA | 2.1 | EF541394 |
| H5HA A/Anhui/1/05/N1NA | 2.3 | DQ0371928 |
| H5HA A/Shenzhen/406H/06/N1NA | 2.3 | EF137706 |
| H5HA A/Hongkong/156/97/N1NA | 0 | AF036356 |

*All pseudotypes use the same N1NA from N/Thailand/1(KAN-1)/04 virus
**The sequence is available from the database of the Los Alancos National Library (http://www.flu.lan/gov/SDN185503 or/SNA 185451)

TABLE III
Quantitative comparison of neutralization titers of two immune sera measured
by MNA and PNA
MNA (CPE: mock infection −; virus ++++)

| | | serum dilution | | | | | | |
|---|---|---|---|---|---|---|---|---|
| Dose of virus | Immunization | 1:10 | 1:20 | 1:40 | 1:80 | 1:160 | 1:320 | 1:640 |
| 100 TCID₅₀ | DNA/LVLP* | −** | − | − | − | ± | + | ++ or +++ |
| | DNA/DNA* | − | − | ± | + | ++ | +++ | ++++ |

PNA

| Dose of | | | | | | | | | | |
|---|---|---|---|---|---|---|---|---|---|---|
| pseudotypes | | serum dillution | | | | | | | | |
| (RLA) | Immunication | 1:10 | 1:20 | 1:40 | 1:80 | 1:160 | 1:320 | 1:640 | 1:1280 | 1:2560 |
| 1 × 10⁶ | DNA/LVLP | 99.9*** | 99.9 | 99.8 | 98.2 | 91.5 | 79.7 | 63.9 | 46.2 | 23.4 |
| | DNA/DNA | 99.9 | 99.9 | 94.6 | 86.9 | 61.5 | 36.0 | 21.7 | 11.3 | 3.4 |
| 2 × 10⁵ | DNA/LVLP | 99.9 | 99.9 | 99.9 | 99.9 | 98.1 | 91.4 | 78.0 | 65.1 | 41.2 |
| | DNA/DNA | 99.9 | 99.9 | 99.6 | 91.9 | 72.0 | 48.0 | 25.3 | 14.4 | 0.1 |
| 4 × 10⁴ | DNA/LVLP | 99.8 | 99.9 | 99.9 | 99.8 | 98.5 | 89.1 | 82.1 | 69.9 | 57.2 |
| | DNA/DNA | 99.9 | 99.0 | 98.8 | 95.7 | 71.5 | 66.3 | 56.9 | 51.2 | 46.1 |

*DNA/LVLP: DNA priming and LVLP boosting: DNA/DNA: DNA priming and DNA boosting
**See FIG. S2 for detailed information of scoring CPE
***% inhibition

TABLE IV
Quantitative comparison of neutralization titers of
immune and challenged ferret sera measured by PNA, MNA and HIA

| | | | PNA | | MNA | HIA |
|---|---|---|---|---|---|---|
| | Immunization | Ferret # | IC50 | IC 95 | titer | titer |
| Immunized | 3.75 μg HA | #6283 | 1:80 | ND* | 1:5 | 1:5 |
| animals | | #6320 | 1:20 | ND | 1:5 | 1:5 |
| | | #6398 | 1:160 | ND | 1:10 | 1:5 |
| | 30 μgHA | #6243 | 1:320 | 1:40-1:80 | 1:40 | 1:40 |
| | | #6278 | 1:160 | ND | 1:5 | 1:5 |
| | | #6371 | 1:640-1:1280 | 1:80-1:180 | 1:10 | 1:20 |
| | 3.75 μg HA + | #6282 | 1:640-1:1280 | 1:180 | 1:80 | 1:40 |
| | AF 03 | #6297 | 1:2560-1:5120 | 1:320 | 1:80 | 1:80 |
| | | #6304 | 1:640-1:1280 | 1:160 | 1:40 | 1:40 |
| | 30 μg | #6319 | 1:2560-1:5120 | 1:320-1:640 | 1:640 | 1:80 |
| | HA + AlOOH | #6397 | 1:2560-1:5120 | 1:640-1:1280 | 1:320 | 1:80 |
| | | #6392 | 1:1280-1:2580 | 1:640-1:1280 | 1:320 | 1:80 |
| | Control AF03 | #6329 | ND | ND | 1:5 | 1:5 |
| | | #6286 | ND | ND | 1:5 | 1:5 |
| | | #6303 | 1:40 | ND | 1:5 | 1:5 |
| | Control AlOOH | #6369 | ND | ND | 1:5 | 1:5 |
| | Control PBS | #6360 | ND | ND | 1:5 | 1:5 |
| Challenged | 3.75 μg HA | #6283 | 1:2560-1:5120 | 1:2560 | 1:1280 | 1:2560 |
| animals | | #6320 | 1:2560-1:5120 | 1:640-1:1280 | 1:320 | 1:1280 |
| | | #6398 | 1:1280-1:2560 | 1:640-1:1280 | 1:640 | 1:640 |
| | 30 μgHA | #6243 | 1:1280-1:2560 | 1:320-1:640 | 1:160 | 1:640 |
| | | #6278 | 1:2560 | 1:640-1:1280 | 1:160 | 1:640 |
| | | #6371 | 1:2560-1:5120 | 1:640 | 1:160 | 1:1280 |
| | 3.75 μg HA + | #6282 | 1:2560-1:5120 | 1:640-1:1280 | 1:1280 | 1:1280 |
| | AF 03 | #6297 | 1:2560 | 1:640-1:1280 | 1:320 | 1:640 |
| | | #6304 | 1:5120-1:10240 | 1:2560-1:5120 | 1:1280 | 1:2560 |
| | 30 μg | #6319 | 1:2560 | 1:320-1:640 | 1:320 | 1:640 |
| | HA + AlOOH | #6397 | <1:10240 | 1:5120-1:10240 | 1:2560 | 1:5120 |
| | | #6392 | 1:5120-1:10240 | 1:1280-1:2560 | 1:1280 | 1:2560 |
| | Control AF03 | #6329 | 1:1280 | 1:40-1:80 | 1:1280 | 1:2560 |
| | | #6286 | ND | ND | 1:5 | 1:5 |
| | | #6303 | 1:80 | ND | 1:5 | 1:5 |
| | Control AlOOH | #6369 | 1:40 | ND | 1:5 | 1:20 |
| | Control PBS | #6360 | 1:20 | ND | 1:5 | 1:20 |

ND: Not detected

TABLE V
Neutralization titers of immune and challenged ferret sera against
pseudotypes expressing seven other H5H4 and one H1HA measured by PNA

| | | | HA/NA pseudotypes | | | |
|---|---|---|---|---|---|---|
| | | | A/Thailand/1 (KAN 1) | | | |
| | | | (04/N1NA | | A/CambodiaP0322085/05/N1NA | |
| | | | Clones of subclones | | | |
| | | | 1 | | 1 | |
| | | Ferret # | IC50 | IC95 | IC50 | IC95 |
| Immunized | 3.75 μg HA | #6263 | 1:20-1:60 | ND* | 1:60-1:160 | ND |
| animals | | #6320 | ND | ND | ND | ND |
| | | #6328 | 1:80 | 1:20 | 1:180 | 1:20-1:60 |
| | 30 μg HA | #6243 | 1:540 | 1:20-1:60 | 1:540-1:1620 | 1:60-1:180 |
| | | #6278 | 1:180-1:540 | ND | 1:180-1:540 | 1:20-1:60 |
| | | #6371 | 1:540-1:1620 | 1:60 | 1:1620-1:4860 | 1:60-1:160 |
| | 3.75 μg | #6282 | 1:540-1:1620 | 1:180 | 1:540-1:1620 | 1:180 |
| | HA-AF03 | #6297 | 1:1620-1:4860 | 1:180-1:540 | 1:1820 | 1:180-1:540 |
| | | #6304 | 1:540-1:1620 | 1:180 | 1:540-1:1620 | 1:180 |
| | 30 μg | #6319 | 1:1620-1:4580 | 1:540-1:1620 | 1:4860 | 1:640 |
| | HA + AlOOH | #6397 | 1:4809 | 1:540-1:1620 | <1:4860 | 1:540-1:1620 |
| | | #6392 | 1:1620-1:4860 | 1:540-1:1620 | 1:1620-1:4860 | 1:540 |
| | Control AF03 | #6329 | ND | ND | ND | ND |
| | | #6288 | ND | ND | ND | ND |
| | | #6303 | ND | ND | 1:20 | ND |
| | Control PBS | #6360 | ND | ND | ND | ND |
| | Control | #6389 | ND | ND | ND | ND |
| | AlOOH | | | | | |
| Challanged | 3.75 μg HA | #6283 | 1:4860 | 1:540-1:1620 | <1:4680 | 1:1620 |
| animals | | #6320 | 1:1620-1:4860 | 1:180-1:540 | 1:4680 | 1:540-1:1620 |
| | | #6398 | <1:4860 | 1:540-1:1620 | 1:1620 | 1:180-1:540 |
| | 30 μg HA | #6243 | 1:1620-1:4860 | 1:180-1:540 | 1:1620-1:4860 | 1:180-1:540 |
| | | #6278 | 1:1620-1:4860 | 1:540-1:1620 | 1:1620-1:4860 | 1:540-1:1620 |
| | | #6371 | 1:1620-1:4860 | 1:540-1:1620 | 1:4860 | 1:540-1:1620 |
| | 3.75 μg | #6282 | 1:1620-1:4860 | 1:540 | <1:4860 | 1:1620 |
| | HA + AF03 | #6297 | 1:1620-1:4860 | 1:540-1:1620 | 1:1620-1:4880 | 1:180-1:540 |
| | | #6304 | <1:4860 | 1:1620 | <1:4060 | 1:1620-1:4860 |
| | 30 μg | #6319 | 1:540-1:1620 | 1:60-1:180 | 1:1620 | 1:180-1:540 |
| | HA + AlOOH | #6397 | <1:4860 | 1:4800 | <1:4880 | <1:4860 |
| | | #6392 | <1:4860 | 1:1620-1:4800 | 1:1620-1:4860 | 1:540-1:1620 |
| | Control AF03 | #6329 | 1:180-1:540 | ND | 1:180-1:640 | 1:20-1:60 |
| | | #6286 | ND | ND | ND | ND |
| | | #6303 | 1:20-1:00 | ND | 1:20-1:00 | ND |
| | Control FBS | #6360 | ND | ND | ND | ND |
| | Control | #6369 | ND | ND | 1:20 | ND |
| | AlOOH | | | | | |
| | | | HA/NA pseudotypes | | | |
| | | | A/CambodiaO0321176/ | | | |
| | | | 06/N1NA | | | |
| | | | Clones of subclones | | | |
| | | | 1 | | H1HAA/WSN/33/N1NA | |
| | | Ferret # | IC50 | IC95 | IC50 | IC95 |
| Immunized | 3.75 μg HA | #6263 | 1:60-1:780 | ND | ND | ND |
| animals | | #6320 | ND | ND | ND | ND |
| | | #6328 | 1:180 | 1:20-1:80 | ND | ND |
| | 30 μg HA | #6243 | 1:1620-1:4380 | 1:180 | ND | ND |
| | | #6278 | 1:180-1:540 | 1:20-1:80 | ND | ND |
| | | #6371 | 1:4660 | 1:100-1:540 | ND | ND |
| | 3.75 μg | #6282 | 1:1820-1:4860 | 1:540 | ND | ND |
| | HA-AF03 | #6297 | 1:4860 | 1:540-1:1620 | ND | ND |
| | | #6304 | 1:1620-1:4860 | 1:540 | ND | ND |
| | 30 μg | #6319 | <1:4860 | 1:1620-1:4860 | ND | ND |
| | HA + AlOOH | #6397 | <1:4860 | 1:1620-1:4860 | ND | ND |
| | | #6392 | <1:4860 | 1:1620 | ND | ND |
| | Control AF03 | #6329 | ND | ND | ND | ND |
| | | #6288 | ND | ND | ND | ND |
| | | #6303 | 1:20 | ND | ND | ND |
| | Control PBS | #6360 | ND | ND | ND | ND |
| | Control | #6389 | ND | ND | ND | ND |
| | AlOOH | | | | | |
| Challanged | 3.75 μg HA | #6283 | <1-4860 | 1:540-1:1620 | 1:540-1:1620 | 1:160-1:540 |
| animals | | #6320 | 1:1820-1:4860 | 1:540-1:1620 | 1:180-1:540 | 1:60-1:160 |
| | | #6398 | 1:1620-1:4860 | 1:540-1:1620 | 1:100 | 1:20-1:60 |
| | 30 μg HA | #6243 | <1:4860 | 1:1620-1:4860 | 1:00 | ND |
| | | #6278 | <1:4860 | 1:1620-1:4860 | 1:540-1:1620 | 1:60 |
| | | #6371 | <1:4880 | 1:1620-1:4860 | 1:180-1:540 | ND |
| | 3.75 μg | #6282 | 1:1620-1:4860 | 1:540-1:1620 | 1:60-1:180 | ND |
| | HA + AF03 | #6297 | 1:1620-1:4860 | 1:540 | 1:180-1:540 | ND |
| | | #6304 | <1:4860 | 1:1620-1:4860 | 1:540 | 1:60-1:180 |
| | 30 μg | #6319 | 1:1820 | 1:180-1:540 | 1:20-1:80 | ND |
| | HA + AlOOH | #6397 | <1:4880 | 1:4860 | 1:540-1:1620 | 1:60-1:180 |
| | | #6392 | 1:1620-1:4860 | ND | 1:180-1:540 | 1:60-1:180 |
| | Control AF03 | #6329 | 1:4860 | 1:60-1:180 | 1:1620 | 1:80-1:180 |
| | | #6286 | ND | ND | 1:180-1:540 | 1:20-1:60 |
| | | #6303 | 1:60 | ND | 1:60 | ND |
| | Control FBS | #6360 | ND | ND | ND | ND |
| | Control | #6369 | 1:20-1:60 | ND | 1:20-1:60 | ND |
| | AlOOH | | | | | |
| | | | HANA pseudotypes | | | |
| | | | A/Indonesia/505/N1NA | | A/Shenzh [symbol] (6/N1NA | |
| | | | clones or subclones | | | |
| | | | 21 | | 23 | |
| | | Ferret # | IC50 | IC95 | IC50 | IC95 |
| Immunized | 37.5 μg HA | #6283 | ND | ND | 1:20 | ND |
| animals | | #3320 | ND | ND | ND | ND |
| | | #8398 | ND | ND | 1:20-1:50 | ND |
| | 30 μg HA | #8243 | 1:60 | ND | 1:60-1:180 | ND |
| | | #6278 | 1:60-1:180 | ND | 1:20-1:60 | ND |
| | | #8371 | 1:540 | ND | 1:540 | ND |
| | 3.75 μg | #6282 | 1:60-1:180 | 1:20 | 1:180-1:540 | 1:20-1:60 |
| | HA + AFCG | #3297 | 1:540 | 1:60 | 1:1620 | 1:80-1:180 |
| | | #6304 | 1:60-1:160 | 1:20 | 1:180-1:540 | 1:20-1:60 |
| | 30 μg | #6319 | 1:540-1:1620 | 1:68-1:180 | 1:1620-1:4860 | 1:180-1:540 |
| | HA − AlOOH | #6397 | 1:620-1:4860 | 1:180-1:540 | 1:1620-1:4860 | 1:180-1:540 |
| | | #6392 | 1:180-1:540 | 1:60-1:180 | 1:1620 | 1:60-1:540 |
| | Control AF03 | #6329 | ND | ND | ND | ND |
| | | #6385 | ND | ND | ND | ND |
| | | #6303 | ND | ND | ND | ND |
| | Control FBS | #6360 | ND | ND | ND | ND |
| | Control | #6369 | ND | ND | ND | ND |
| | AlOOH | | | | | |
| Changed | 3.75 μHA | #6283 | <1:4860 | 1:540-1:1620 | 1:1620-1:4860 | 1:180-1:540 |
| animals | | #6320 | 1:540-1:1620 | 1:60-1:180 | 1:540:1:1620 | 1:180-1:540 |
| | | #6398 | 1:180-1:540 | ND | 1:540 | 1:60 |
| | 30 μg HA | #6243 | 1:620-1:4860 | 1:60-1:180 | 1:540 | 1:60-1:180 |
| | | #6278 | 1:540-1:1620 | 1:180-1:540 | 1:1620 | 1:180 |
| | | #6371 | 1:540-1:1620 | 1:180-1:540 | 1:1620-1:4860 | 1:180-1:540 |
| | 3.75 μg | #6282 | <1:4860 | 1:540-1:160 | 1:4860 | 1:540-1:1620 |
| | HA + AF03 | #6297 | 1:4860 | 1:180-1:540 | 1:540-1:1620 | 1:60-1:540 |
| | | #6304 | <1:4880 | 1:1620-1:4860 | <1:4860 | 1:540-1:1620 |
| | 30 μg | #6319 | 1:540-1:1620 | 1:160-1:540 | 1:540-1:1620 | 1:60-1:180 |
| | HA + AlOOH | #6397 | <1:4860 | 1:1620 | <1:4860 | 1:1620-1:14860 |
| | | #6392 | 1:1620-1:4860 | 1:180-1:540 | 1:1620-1:4860 | 1:540-1:1620 |
| | Control AF03 | #6329 | 1:540 | ND | 1:60-1:180 | ND |
| | | #6386 | ND | ND | ND | ND |
| | | #6303 | ND | ND | ND | ND |
| | Control PBS | #6360 | ND | ND | ND | ND |
| | Control | #6389 | ND | ND | ND | ND |
| | AlOOH | | | | | |
| | | | HANA pseudotypes | | | |
| | | | Ar [symbol] /1/0/N1NA | | A/Honcogkong/150/97/N1NA | |
| | | | clones or subclones | | | |
| | | | 23 | | 0 | |
| | | Ferret # | IC50 | IC95 | IC50 | IC25 |
| Immunized | 37.5 μg HA | #6283 | ND | ND | 1:180 | ND |
| animals | | #3320 | ND | ND | 1:20-1:60 | ND |
| | | #8398 | 1:20 | ND | 1:180 | ND |
| | 30 μg HA | #8243 | 1:20-1:60 | ND | 1:60-1:180 | ND |
| | | #6278 | 1:20-1:60 | ND | 1:60-1:180 | ND |
| | | #8371 | 1:60-1:180 | ND | 1:180 | 1:20-1:60 |
| | 3.75 μg | #6282 | 1:510 | 1:120-1:160 | 1:180-1:540 | ND |
| | HA + AFCG | #3297 | 1:540-1:1620 | 1:20-1:60 | 1:180-1:540 | 1:20-1:60 |
| | | #6304 | 1:180 | 1:20 | 1:180 | ND |
| | 30 μg | #6319 | 1:1620 | 1:160 | 1:180-1:540 | 1:60-1:80 |
| | HA − AlOOH | #6397 | 1:1620 | 1:180-1:540 | 1:180-1:540 | 1:180 |
| | | #6392 | 1:540-1:1620 | 1:60-1:180 | 1:180-1:540 | 1:60-1:180 |
| | Control AF03 | #6329 | ND | ND | ND | ND |
| | | #6385 | ND | ND | ND | ND |
| | | #6303 | ND | ND | ND | ND |
| | Control FBS | #6360 | ND | ND | ND | ND |
| | Control | #6369 | ND | ND | ND | ND |
| | AlOOH | | | | | |
| Changed | 3.75 μHA | #6283 | 1:540-1:1620 | 1:180-1:540 | 1:4960 | 1:180-1:540 |
| animals | | #6320 | 1:540-1:1620 | 1:180 | 1:540-1:1620 | 1:180-1:540 |
| | | #6398 | 1:540-1:1620 | 1:180 | 1:540-1:1620 | 1:180-1:540 |
| | 30 μg HA | #6243 | 1:180-1:540 | 1:60-1:180 | 1:540-1:1620 | 1:180-1:540 |
| | | #6278 | 1:180-1:540 | 1:60-1:180 | 1:540-1:1620 | 1:180-1:540 |
| | | #6371 | 1:1620 | 1:180-1:540 | 1:540-1:1620 | 1:180-1:540 |
| | 3.75 μg | #6282 | <1:4860 | 1:540-1:1620 | 1:1620 | 1:180-1:540 |
| | HA + AF03 | #6297 | 1:180-1:540 | 1:180 | 1:540 | 1:180-1:540 |
| | | #6304 | <1:4860 | 1:540-1:1620 | <1:4860 | 1:540-1:1620 |
| | 30 μg | #6319 | 1:540 | 1:60-1:180 | 1:540 | 1:60-1:180 |
| | HA + AlOOH | #6397 | <1:4860 | 1:1620-1:4860 | 1:4860 | 1:540-1:1620 |
| | | #6392 | 1:1620-1:4860 | 1:540 | 1:1620 | 1:180-1:540 |
| | Control AF03 | #6329 | 1:60 | ND | 1:540 | ND |
| | | #6386 | ND | ND | ND | ND |
| | | #6303 | ND | ND | ND | ND |
| | Control PBS | #6360 | ND | ND | ND | ND |
| | Control | #6389 | ND | ND | 1:20 | ND |
| | AlOOH | | | | | |

ND: Not detected [symbol]
indicates data missing or illegible when filed

TABLE VI
comparison of HIA, MNA and PNA for measuring neutralizing
antibodies against HPAI H5N1 viruses

| | HIA | MNA | PNA |
|---|---|---|---|
| Epitope | binding to RBC | virus entry | virus entry |
| involves | | | |
| Virus | wild type | wild type | pseudotypes |
| Virus particles | infectious and | infectious | infectious |
| measured | non-infectious | | |
| Target cells | RBC | MDCK, others | MDCK, others |
| Biocontain | BL-3 | BL-3 | BL-2 |
| Readout | HI | CPE | luciferase activity |
| | semi-quantifiable | semi-quantifiable | quantifiable |
| | subjective | subjective | objective |
| Time required | within one day | 5-6 days | 2-3 days |
| for assay | | | |

### REFERENCES

1. Kobinger G P, Weiner D J, Yu Q C, Wilson J M. Filovirus-pseudotyped lentiviral vector can efficiently and stably transduce airway epithelia in vivo. Nat Biotechnol. 2001; 19:225-230.
2. Medina M F, Kobinger G P, Rux J, Gasmi M, Looney D J, Bates P, Wilson J M. Lentiviral vectors pseudotyped with minimal filovirus envelopes increased gene transfer in murine lung. Mol Ther. 2003; 8:777-789.
3. Chandran K, Sullivan N J, Felbor U, Whelan S P, Cunningham J M. Endosomal proteolysis of the Ebola virus glycoprotein is necessary for infection. Science 2005; 308:1643-1645.
4. Szecsi J, Boson B, Johnsson P, Dupeyrot-Lacas P, Matrosovich M, Klenk H D, Klatzmann D, Volchkov V, Cosset F L. Induction of neutralizing antibodies by virus-like particles harbouring surface proteins from highly pathogenic H5N1 and H7N1 influenza viruses. J. Virol. 2006 (advanced online publication)
5. Liu Y, Deisseroth A. Tumor vascular targeting therapy with viral vectors. Blood 2006; 107:3027-3033.
6. Richman D D, Wrin T, Little S J, Petropoulos C J. Rapid evolution of the neutralizing antibody response to HIV type 1 infection. Proc Natl Acad Sci USA. 2003; 100(7):4144-4149.
7. Frost S D, Wrin T, Smith D M, Kosakovsky Pond S L, Liu Y, Paxinos E, Chappey C, Galovich J, Beauchaine J, Petropoulos C J, Little S J, Richman D D. Neutralizing antibody responses drive the evolution of human immunodeficiency virus type 1 envelope during recent HIV infection. Proc Natl Acad Sci USA. 2005; 102(51):18514-18519.
8. Naldini L, Blomer U, Gallay P, Ory D, Mulligan R, Gage F H, Verma I M, Trono D. In vivo gene delivery and stable transduction of nondividing cells by a lentiviral vector. Science 1996; 272:263-267.
9. Zufferey R, Nagy D, Mandel R J, Naldini L, Trono D. Multiply attenuated lentiviral vector achieves efficient gene delivery in vivo. Nat Biotechnol. 1997; 15:871-875.
10. Biffi A, Naldini L. Gene therapy of storage disorders by retroviral and lentiviral vectors. Hum Gene Ther. 2005; 16:1133-1142.
11. McKay T, Patel M, Pickles R J, Johnson L G, Olsen J C. Influenza M2 envelope protein augments avian influenza hemagglutinin pseudotyping of lentiviral vectors. Gene Ther. 2006; 13:715-724.
12. Duisit G, Conrath H, Saleun S, Folliot S, Provost N, Cosset F L, Sandrin V, Moullier P, Rolling F. Five recombinant simian immunodeficiency virus pseudotypes lead to exclusive transduction of retinal pigmented epithelium in rat. Mol Ther. 2002; 6:446-454.
13. Kobayashi M, Iida A, Ueda Y, Hasegawa M. Pseudotyped lentivirus vectors derived from simian immunodeficiency virus SIVagm with envelope glycoproteins from paramyxovirus. J Virol. 2003; 77:2607-2614.
14. Bartosch B, Dubuisson J, Cosset F L. Infectious hepatitis C virus pseudo-particles containing functional E1-E2 envelope protein complexes. J Exp Med. 2003; 197:633-642.
15. Kolokoltsov A A, Weaver S C, Davey R A. Efficient functional pseudotyping of oncoretroviral and lentiviral vectors by Venezuelan equine encephalitis virus envelope proteins. J Virol. 2005; 79:756-763.
16. Wiley D C, Skehel J J. Crystallization and x-ray diffraction studies on the haemagglutinin glycoprotein from the membrane of influenza virus. J Mol Biol 1977; 112:343-347.
17. Wilson I A, Skehel J J, Wiley D C. Structure of the haemagglutinin membrane glycoprotein of influenza virus at 3 resolution. Nature 1981; 289:366-373.
18. Rogers G N, Paulson J C, Daniels R S, et al. Single amino acid substitutions in influenza haemagglutinin change receptor binding specificity. Nature 1983; 304:76-78.
19. Weis W, Brown J H, Cusack S, et al. Structure of the influenza virus haemagglutinin complexed with its receptor, sialic acid. Nature 1988; 333:426-431.
20. Dopheide T A, Ward C W. The carboxyl-terminal sequence of the heavy chain of a Hong Kong influenza hemagglutinin. Eur J Bioche. 1978; 85:393-398.
21. Garten W, Klenk H-D. Characterization of the carboxypeptidase involved in the proteolytic cleavage of the influenza haemagglutinin. Intervirology 1983; 20:181-189.
22. Klenk H-D, Rott R, Orlich M, Blodorn J. Activation of influenza A viruses by trypsin treatment. Virology 1975; 68:426-439.
23. Lazarowitz S G, Choppin P W. Enhancement of the infectivity of influenza A and B viruses by proteolytic cleavage of the hemagglutinin polypeptide. Virology 1975; 68:440-454.
24. Gotoh B, Ogasawara T, Toyoda T, et al. An endoprotease homologous to the blood clotting factor X as a determinant of viral tropism in chick embryo. EMBO J 1990; 9: 4189-4195.
25. Gottschalk A. The specific enzyme of influenza virus and Vibrio cholerae. Biochim Biophys Acta 1957; 23: 645-646.
26. Palese P, Tobita K, Ueda M, Compans R W. Characterization of temperature sensitive influenza virus mutants defective in neuraminidase. Virology 1974; 61:397-410.
27. Kobasa D, Rodgers M E, Wells K, Kawaoka Y. Neuraminidase hemadsorption activity, conserved in avian influenza A viruses, does not influence viral replication in ducks. J Virol 1997; 71: 6706-6713.
28. Laver W G, Colman P M, Webster R G, et al. Influenza virus neuraminidase with hemagglutinin activity. Virology 1984; 137:314-323.
29. Matrosovich M N, Matrosovich T Y, Gray T, Roberts N A, Klenk H D. Neuraminidase is important for the initiation of influenza virus infection in human airway epithelium. J Virol. 2004; 78:12665-12667.
30. Lamb R A, Zebedee S L, Richardson C D. Influenza virus M2 protein is an integral membrane protein expressed on the infected-cell surface. Cell 1985; 40: 627-633.
31. Lamb R A, Holsinger L J, Pinto L H. The influenza A virus M₂ ion channel protein and its role in the influenza virus life cycle, In: Wimmer E, ed. Receptor-mediated virus entry into cells. Cold Spring Harbor, N.Y.: Cold Spring Harbor Press, 1994:303-321.
32. Ohuchi M, Cramer A, Vey M, et al. Rescue of vector-expressed fowl plague virus hemagglutinin in biologically active form by acidotropic agents and coexpressed M2 protein. J Virol. 1994; 68:920-926.
33. Hatziioannou T, Valsesia-Wittmann S, Russell S J, Cosset F L. Incorporation of fowl plague virus hemagglutinin into murine leukemia virus particles and analysis of the infectivity of the pseudotyped retroviruses. J Virol. 1998; 72:5313-5317.
34. Horimoto T, Kawaoka Y. Influenza: lessons from past pandemics, warnings from current incidents. Nat Rev Microbiol. 2005; 3:591-600.
35. Shinya K, Ebina M, Yamada S, Ono M, Kasai N, Kawaoka Y Avian flu: influenza virus receptors in the human airway. Nature 2006; 440:435-436.
36. Naldini L, Blomer U, Gage F H, Trono D, Verma I M. Efficient transfer, integration, and sustained long-term expression of the transgene in adult rat brains injected with a lentiviral vector. Proc Natl Acad Sci U S A. 1996; 93:11382-11388.
37. Prodromou C, Pearl L H. Recursive PCR: a novel technique for total gene synthesis. Protein Eng. 1992; 5(8):827-9.
38. Manthorpe M, Cornefert-Jensen F, Hartikka J, Felgner J, Rundell A, Margalith M, Dwarki V. Gene therapy by intramuscular injection of plasmid DNA: studies on firefly luciferase gene expression in mice. Hum Gene Ther. 1993; 4:419-31.
40. Zhou P, Goldstein S, Devadas K, Tewari D, Notkins A L. Human CD4+ cells transfected with IL-16 cDNA are resistant to HIV-1 infection:inhibition of mRNA expression. Nat Med. 1997; 3:659-664.
41. Peiris, J. S. M., de Jong, M. D., Guan, Y. Avian Influenza Virus (H5N1): a Threat to Human Health. Clin. Microbiol. Rev. 20:243-267 (2007).
42. WHO Cumulative number of Confirmed Human Cases of Avian Influenza A (H5N1) Reported to WHO. http://www.who.int/cst/disease/avian_influenza/country/cases_table_{—}2008_{—}06_{—}19/en/ind ex.html (2008).
43. Rowe, T. et al. Detection of antibody to avian influenza A (H5N1) virus in human serum by using a combination of serologic assays. J. Clin. Microbiol. 37:937-943 (1999).
44. World Health Organization. Collecting, preserving and shipping specimens for the diagnosis of avian influenza A (H5N1) virus infection: guide for field operations. (Accessed Dec. 20, 2007, at http//www.who.int/csr/resources/publications/surveillance/whocdscsredc2004.pdf.) (2007).
45. Stephenson, I., Wood, J. M., Nicholson, K. G. and Zambon, M. C. Sialic acid receptor specificity on erythrocytes affects detection of antibody to avian influenza hemagglutinin. J. Med. Virol. 70:391-398 (2003).
46. Reiser, J. Production and concentration of pseudotyped HIV-1-based gene transfer vectors. Gene Ther. 7:910-913 (2000).
47. Watson, D. J., Kobinger, G. P., Passini, M. A., Wilson, J. M. and Wolfe, J. H. Targeted transduction patterns in the mouse brain by lentivirus vectors pseudotyped with VSV, Ebola, Mokola, LCMV, or MuLV envelope proteins. Mol Ther. 5:528-37 (2002).
48. Kong, W. P. et al. Protective immunity to lethal challenge of the 1918 pandemic influenza virus by vaccination. Proc. Natl. Acad. Sci. USA 103:15987-15991 (2006).
49. Bartosch, B., Dubuisson, J. and Cosset, F. L. Infectious hepatitis C virus pseudoparticles containing functional E1-E2 envelope protein complexes. J. Exp. Med. 197:633-642 (2003).
50. Simmons, G. et al. Characterization of severe acute respiratory syndrome-associated coronavirus (SARS-CoV) spike glycoprotein-mediated viral entry. Proc. Natl. Acad. Sci. USA 101:4240-4245 (2004).
51. Duisit, G. et al. Five recombinant simian immunodeficiency virus pseudotypes lead to exclusive transduction of retinal pigmented epithelium in rat. Mol. Ther. 6:446-454 (2002).
52. McKay, T., Patel, M., Pickles, R. J., Johnson, L. G. and Olsen, J. C. Influenza M2 envelope protein augments avian influenza hemagglutinin pseudotyping of lentiviral vectors. Gene Ther. 13:715-724 (2006).
53. Cosset, F. L. et al. Characterization of Lassa virus cell entry and neutralization with Lassa pseudotypes. J. Virol. Advance online publication (Jan. 19, 2009).
54. Nefkens, I. et al. Hemagglutinin pseudotyped lentiviral particles: characterization of a new method for avian H5N1 influenza sero-diagnosis. J. Clin. Virol. 39:27-33 (2007)
55. Yang, Z. Y. et al. Immunization by avian H5 influenza hemagglutinin mutants with altered receptor binding specificity. Science 317:825-828 (2007).
56. Writing Committee of the Second World Health Organization Consultation on clinical Aspects of Human Infection with Avian Influenza A (H5N1) Virus. Update on Avian Influenza A (H5N1) Virus Infection in Human. N. Engl. J. Med. 358:261-273 (2008).
57. Hoffmann, E., Lipatov, A. S., Webby, R. J., Govorkova, E. A. and Webster, R. G. Role of specific hemagglutinin amino acids in the immunogenicity and protection of H5N1 influenza virus vaccines. Proc. Natl. Acad. Sci. USA 102:12915-12920 (2005).
58. Berger, E. A., Murphy, P. M. and Farber, J. M. Chemokine receptors as HIV-1 coreceptors: roles in viral entry, tropism, and disease. Annu. Rev. Immunol. 17:657-700 (1999).
59. Skehel, J. J. and Wiley, D. C. Receptor binding and membrane fusion in virus entry: the influenza hemagglutinin. Annu. Rev. Biochem. 69:531-569 (2000).
60. Horimoto, T. and Kawaoka, Y. Strategies for developing vaccines against H5N1 influenza A viruses. Trends in Mol. Med. 12:506-514 (2006).
61. Throsby, M. et al. Heterosubtypic neutralizing monoclonal antibodies cross-protective against H5N1 and H1N1 recovered from human IgM⁺ memory B cells. PloS One 3(12):e3942 (2008).
62. Kashyap, A. K. et al. Combinatorial antibody libraries from survivors of the Turkish H5N1 avian influenza outbreak reveal virus neutralization strategies. Proc. Natl. Acad. Sci. USA 105:5986-5991 (2008).
63. Sui, J. et al. Structural and functional bases for broad-spectrum neutralization of avian and human influenza A viruses. Nat. Structural Biol. Advance online publication (Feb. 22, 2009).
64. Deng, H-K. et al. Identification of a major co-receptor for primary isolates of HIV-1. Nature 381:661-666 (1996)
65. Follenzi, A. and Naldini, L. Generation of HIV-1 derived lentiviral vectors. Methods Enzymol. 346:454-465 (2002).
66. Manthorpe, M. et al. Gene therapy by intramuscular injection of plasmid DNA: studies on firefly luciferase gene expression in mice. Hum Gene Ther. 4:419-431 (1993).
67. Hoffmann, E., Stech, J., Guan, Y., Webster, R. G, and Perez, D. R. Universal primer set for the full-length amplification of all influenza A viruses. Arch. Virol. 146:2275-2289 (2001).
68. Luo, G, Chung, J. and Palese, P. Alterations of the stalk of the influenza virus neuraminidase: deletions and insertions. Virus Res. 29:141-153 (1993).
69. Dong, J., Roth, M. G. and Hunter, E. A chimeric avian retrovirus containing the influenza virus hemagglutinin gene has an expanded host range. J. Virol. 66:7374-7382. (1992).
70. U.S. Pat. No. 7,402,436.

1. A lentivirus vector pseudotyped with:
an influenza HA protein or a protein containing an HA protein fragment comprising an HA epitope or an HA cellular attachment ligand,
wherein said HA protein is not fowl plague virus H7HA.

2. The lentivirus vector of claim 1, wherein said HA protein is selected from the group consisting of H1, H2, H5 and H7.

3. The lentivirus vector of claim 1 or 2, wherein said HA protein is selected from the group consisting of: SEQ ID NO: 1, SEQ ID NO: 3, SEQ ID NO: 4, SEQ ID NO: 5, SEQ ID NO: 6, SEQ ID NO: 7, SEQ ID NO: 12, SEQ ID NO: 13.

4. The lentivirus vector of claim 1, 2 or 3, further comprising NA.

5. The lentivirus vector of claim 1, 2, 3 or 4, further comprising NA from an H5N1 avian flu strain.

6. The lentivirus vector of claim 4 or 5, wherein said NA is selected from the group consisting of: SEQ ID NO: 8, SEQ ID NO: 9, SEQ ID NO: 10, SEQ ID NO: 11.

7. The lentivirus vector of any one of claims 1 to 6, further comprising NA and M2.

8. The lentivirus vector of any one of claims 1 to 7, further comprising a transgene.

9. The lentivirus vector of any one of claims 1 to 8, wherein the polynucleotide expressing HA has been codon-optimized for a target or host cell.

10. The lentivirus vector of any one of claims 1 to 9, wherein the polynucleotides expressing HA, and NA and M2 if present, have been codon-optimized for a target or host cell.

11. The lentivirus vector of anyone of claims 1 to 10, wherein said HA protein consists of at least two portions from different HA homologues.

12. The lentivirus vector of anyone of claims 3 to 10, wherein said NA protein consists of at least two portions from different NA homologues.

13. A composition comprising the lentivirus vector of any one of claims 1 to 12 and a pharmaceutically acceptable excipient, carrier and/or immunological adjuvant.

14. A lentivirus vector packaging system comprising:
at least one packaging vector expressing HA, and
a transfer vector construct comprising production and packaging sequences, sequences expressing the Gag and Pol lentivirus proteins, and optionally a transgene,
wherein said HA protein is not H7HA.

15. A lentivirus vector packaging system comprising:
at least one packaging vector expressing HA,
a helper construct expressing the Gag and Pol lentivirus proteins, and
a transfer vector construct comprising production and packaging sequences and optionally a transgene;
wherein said HA protein is not H7HA.

16. A method for inducing an immune response comprising administering the lentivirus vector of any one of claims 1 to 12, to a subject in an amount sufficient to induce an immune response to said vector.

17. A method for identifying a neutralizing antibody comprising:
contacting the lentivirus vector of any one of claims 1 to 12, with an antibody for a time and under conditions suitable for binding of the antibody to the lentivirus vector, and
determining the effects of said contact on the ability of said lentivirus vector to bind to or infect a host cell.

18. A target or host cell transfected with the lentivirus vector of any one of claims 1 to 12.

19. A composition comprising the target or host cell of claim 18 and a pharmaceutical acceptable excipient, carrier and/or immunological adjuvant.

20. A target or host cell transfected with the lentivirus vector of claim 8, wherein said transgene has been incorporated into the chromosomal DNA of said cell.

21. A method for transducing a polynucleotide sequence or a transgene into a cell comprising contacting a cell with the lentivirus vector of claim 8 for a time and under conditions sufficient for transduction.

22. A method for identifying a molecule that modulates virus binding to a cell or which modulates viral infection of a cell comprising:
contacting a cell with a candidate molecule and the pseudotyped lentivirus of any one of claims 1 to 12, and
determining the ability of said candidate molecule to modulate virus binding to said cell or to inhibit viral infection of the cell.

23. The method of claim 22, wherein said molecule is a non-protein drug.

24. The method of claim 22, wherein said molecule is a peptide or polypeptide which is not an antibody.

25. The method of claim 22, wherein said molecule is an antibody.

26. The method of claim 22, wherein said molecule comprises a carbohydrate or lipid.

27. A method of using a lentivirus vector as claimed in claim 8 to transfect at least one target cell, wherein said HA, NA and M2 are used in the ratio 8:2:1.

28. A pseudotyped Lentivirus vector based neutralization assay comprising the steps of:
a) bringing into contact a first population of cells with
at least one Lentivirus vector comprising a marker and pseudotyped with one or more antigens selected from the group: an influenza HA protein or a protein containing an HA protein fragment comprising an HA epitope or an HA cellular attachment ligand and an influenza NA protein, a protein containing an NA protein fragment comprising or an NA epitope and
a sample of sera;
b) incubating said first population of cells with said at least one pseudotyped Lentivirus vector and said sera;
c) determining the presence of said marker in said population of cells.

29. The method of claim 28, wherein said at least one Pseudotyped Lentivirus vector comprises a set of Lentivirus vectors which have been pseudotyped with a panel of different influenza HA and/or influenza NA antigens.

30. The method of claim 28, comprising comparing the detected level of said marker in said first cell population to the level of said marker in a control cell population exposed to one element selected from the group: said at least one Pseudotyped Lentivirus vector and said sera.


SEQUENCE LISTING (45 sequences)

SEQ ID NO: 1 - protein, 566 aa, Influenza virus
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSDYPVAKGSYNNTSGEQMLIIWGVHHPNDETEQRTLYQNVGTYVSVSTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRRKKRGLFGAIAGFIEGGWQGMVDGWYGYHHSNDQGSGYAADKESTQKAFDGITNKVNSVIEKMNTQFEAVGKEFGNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 2 - DNA, 1039 bp, Influenza virus
agcaaaagcaggtagatgttgaaagatgagtcttctaaccgaggtcgaaacgtacgttctctctatcatcccgtcaggccccctcaaagccgagatcgcgcagaaacttgaagatgtctttgcaggaaagaacaccgatctcgaggctctcatggagtggctaaagacaagaccaatcctgtcacctctgactaaagggattttgggatttgtattcacgctcaccgtgcccagtgagcgaggactgcagcgtagacgctttgtccagaatgccctaaatggaaatggagatccaaataatatggatagagcagtcaagctatataagaagctgaaaagagaaataacattccatggggctaaggaggtcgcactcagctactcaaccggtgcacttgccagttgcatgggtctcatatacaacaggatgggaacggtgactacggaagtggcttttggcctagtgtgtgccacttgtgagcagattgcagattcacagcatcggtctcacagacagatggcaactaccaccaacccactaatcagacatgagaacagaatggtgctggccagcactacagctaaggctatggagcagatggcaggatcaagtgagcaggcagcggaagccatggagatcgctaatcaggctaggcagatggtgcaggcaatgaggacaattgggactcatcctaactctagtgctggtctgagagataatcttcttgaaaatttgcaggcctaccagaaacgaatgggagtgcagatgcagcgattcaagtgatcctattgttgttgccgcaaatatcattgggatcttgcacttgatattgtggattcttgatcgtcttttcttcaaatgcatttatcgtcgccttaaatacggtttgaaaagagggcctgctacggcaggggtacctgagtctatgagggaagagtaccggcaggaacagcagagtgctgtggatgttgacgatggtcattttgtcaacatagaattggagtaaaaaactaccttgtttctactaatacggaagac

SEQ ID NO: 3 - protein, 569 aa, Influenza virus
MKAKLLVLLYAFVATDADTICIGYHANNSTDTVDTIFEKNVAVTHSVNLLEDRHNGKLCKLKGKAPLQLGKCSITGWLLGNPECDSLLPARSWSYIVETPNSENGACYPGDFIDYEELREQLSSVSSLERFEIFPKESSWPNHTFNGVTVSCSHRGKSSFYRNLLWLTKEGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRRKKRGLFGAIAGFIEGGWTGMIDGWYGYHHQNEQGSGYAADQKSTQNAINGITNKVNSIIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLKCRICI

SEQ ID NO: 4 - protein, 568 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 5 - protein, 568 aa, Influenza virus
MEKIVLLFAVVSLVKSDQICIGYHANNSTEQVDTIMEKNVTVTHAQDILERTHNGKLCDLDGVKPLILRDCSVAGWLLGNPMCDEFINVPEWSYIVERANPVNGLCYPGDFNDYEELKHLLSRINHFEKIQIIPKSSWSSHEASLGVSSACPYLGKPSFFRNVVWLIKKNSTYPTIKRSYNNTNQEDLLVMWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMGNERTLDFHDSNVKNLYDKVRLQLRDNAKELGNGCFEFYHKCDNECMESVRNGTYDYPQYSEEARLKREEISGVKLESIGIYQILSIYSTVASSLALAIMIAGLSLWMCSNGSLQCRICI

SEQ ID NO: 6 - protein, 567 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQGKSSFFRNVVWLIKKNSTYPTIKRSYNNTNQEDLLVMWGIHHPNDEAEQTKLYQNPITYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 7 - protein, 567 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIPIIPKSSWSDHEASSGVSSACPYQGTPSFFRNVVWLIKKNNTYPTIKRSYNNTNQEDLLILWGIHHSNNAAEQTKLYQNPTTYISVGTSTLNLRLVPKIATRSKVNGQSGRMDFFWTILKPNDAINFESNGNFIAPEYAYKIVKKGDSAIMKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 8 - protein, 449 aa, Influenza virus
MNPNKKIITIGSICMVTGMVSLMLQIGNLISIWVSHSIHTGNQHKAEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 9 - protein, 449 aa, Influenza virus
MNPNQKIITIGSICMITGMVSLMLQVGNMISIWISHSIHTGNQHQAEPISNANFLTKRAVAAVKLAGNSSLCPINGWAVYSKDNSIRIGSKGDVFVIREPFISCSHLECRTFFLTQGALLNDKHSNGTAKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWIELIRGRPKESTIWTSGSSISFCGVNSDTVGWSWPDGAELPFTIDK

SEQ ID NO: 10 - protein, 449 aa, Influenza virus
MNPNQKIITIGSICMVIGIVSLMLQIGNMVSLWVSHSIQTGNQHQVEPISNTNFLTEKAVASVTLAGNSSLCPIRGWAVHSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGMITDTIKSWRNNILRTQESECACVNGSCFTVMTDGPSNGQASYKIFKMEKGKVVKSVELDAPNYHYEECSCYPDAGEITCVCRDNWHGSNRPWVSFNQNLEYQIGYICSGVFGDNPRPNGGTGSCGPVSPNGAYGVKGFSFKYGNGVWIGRTKSPSSRSGFEMIWDPNGWTETDSSFSVKQDIVAITDWSGYSGSFVQHPELTGLDCIRPCFWVELIRGRPKESTIWTSGSSISFCGVNSDTVSWSWPDGAELPFTIDK

SEQ ID NO: 11 - protein, 449 aa, Influenza virus
MNPNQKIITIGSICMVIGIVSLMLQIGNMISIWVSHSIQTGNQHQAEPISNTNFLTENAVASVTLAGNSSLCPIRGWAVHSKDNSIRIGSKGDVFVIREPFISCSHLECRTFFLTQGALLNDKHSNGTVKDRSPHGTLMSCPMGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELNAPNYHYEECSCYPDAGEIICVCRDNWHGSNRPWVSFNQNLEYQIGYICSGVFGDNPRPNDGTGSCGPVSPNGAYGIKGFSFKYGNGVWIGRTKSTNSRSGFEMIWDPNGWTGTDSDFSVKQDIVAITDWSGYSGSFVQHPELTGLDCIRPCFWVELIRGRPKESTIWTSGSSISFCGVNSDTVSWSWPDGAELPFTIDK

SEQ ID NO: 12 - protein, 569 aa, Artificial
  Chimeric HA peptide
MKAKLLVLLYAFVATDADTICIGYHANNSTDTVDTIFEKNVAVTHSVNLLEDRHNGKLCKLKGKAPLQLGKCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPQGSINSNLPFQNIHPVTIGECPKYVRSTKLRMVTGLRNIPSIQYRRKKRGLFGAIAGFIEGGWTGMIDGWYGYHHQNEQGSGYAADQKSTQNAINGITNKVNSIIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLKCRICI

SEQ ID NO: 13 - protein, 568 aa, Artificial
  Chimeric HA peptide
MEKIVLLFAIVSLVKSDQICIGYHANNSTEQVDTIMEKNVTVTHAQDILEKTHNGKLCDLDGVKPLILRDCSITGWLLGNPECDSLLPARSWSYIVETPNSENGACYPGDFIDYEELREQLSSVSSLERFEIFPKESSWPNHTFNGVTVSCSHRGKSSFYRNLLWLTKEGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 14 - DNA, 1707 bp, Artificial
  Coding Seq for H151HA
atgaaggccaagctgctggtgctgctgtacgccttcgtggccaccgacgccgacaccatctgcatcggctaccacgccaacaacagcaccgacaccgtggacaccatcttcgagaagaacgtggccgtgacccacagcgtgaacctgctggaggacagacacaacggcaagctgtgcaagctgaagggcaaggcccccctgcagctgggcaagtgcagcgtggccggctggctgctgggcaaccccatgtgcgacgagttcatcaacgtgcccgagtggagctacatcgtggagaaggccaaccccgtgaacgacctgtgctaccccggcgacttcaacgactacgaggagctgaagcacctgctgagcagaatcaaccacttcgagaagatccagatcatccccaagagcagctggagcagccacgaggccagcctgggcgtgagcagcgcctgcccctaccagagaaagagcagcttcttcagaaacgtggtgtggctgatcaagaagaacagcacctaccccaccatcaagagaagctacaacaacaccaaccaggaggacctgctggtgctgtggggcatccaccaccccaacgacgccgccgagcagaccaagctgtaccagaaccccaccacctacatcagcgtgggcaccagcaccctgaaccagagactggtgcccagaatcgccaccagaagcaaggtgaacggccagagcggcagaatggagttcttctggaccatcctgaagcccaacgacgccatcaacttcgagagcaacggcaacttcatcgcccccgagtacgcctacaagatcgtgaagaagggcgacagcaccatcatgaagagcgagctggagtacggcaactgcaacaccaagtgccagaccccccagggcagcatcaacagcaacctgcccttccagaacatccaccccgtgaccatcggcgagtgccccaagtacgtgagaagcaccaagctgagaatggtgaccggcctgagaaacatccccagcatccagtacagaagaaagaagagaggcctgttcggcgccatcgccggcttcatcgagggcggctggaccggcatgatcgacggctggtacggctaccaccaccagaacgagcagggcagcggctacgccgccgaccagaagagcacccagaacgccatcaacggcatcaccaacaaggtgaacagcatcatcgagaagatgaacacccagttcaccgccgtgggcaaggagttcaacaacctggagaagagaatggagaacctgaacaagaaggtggacgacggcttcctggacatctggacctacaacgccgagctgctggtgctgctggagaacgagagaaccctggacttccacgacctgaacgtgaagaacctgtacgagaaggtgaagagccagctgaagaacaacgccaaggagatcggcaacggctgcttcgagttctaccacaagtgcgacaacgagtgcatggagagcgtgagaaacggcacctacgactaccccaagtacagcgaggagagcaagctgaacagagagaagatcgacggcgtgaagctggagagcatgggcgtgtaccagatcctggccatctacagcaccgtggccagcagcctggtgctgctggtgagcctgggcgccatcagcttctggatgtgcagcaacggcagcctgaagtgcagaatctgcatc

SEQ ID NO: 15 - DNA, 1704 bp, Artificial
  H515HA DNA Coding SEQ
atggagaagatcgtgctgctgttcgccatcgtgagcctggtgaagagcgaccagatctgcatcggctaccacgccaacaacagcaccgagcaggtggacaccatcatggagaagaacgtgaccgtgacccacgcccaggacatcctggagaagacccacaacggcaagctgtgcgacctggacggcgtgaagcccctgatcctgagagactgcagcatcaccggctggctgctgggcaaccccgagtgcgacagcctgctgcccgccagaagctggagctacatcgtggagacccccaacagcgagaacggcgcctgctaccccggcgacttcatcgactacgaggagctgagagagcagctgagcagcgtgagcagcctggagagattcgagatcttccccaaggagagcagctggcccaaccacaccttcaacggcgtgaccgtgagctgcagccacagaggcaagagcagcttctacagaaacctgctgtggctgaccaaggagggcgacagctaccccaagctgaccaacagctacgtgaacaacaagggcaaggaggtgctggtgctgtggggcgtgcaccaccccagcagcagcgacgagcagcagagcctgtacagcaacggcaacgcctacgtgagcgtggccagcagcaactacaacagaagattcacccccgagatcgccgccagacccaaggtgaaggaccagcacggcagaatgaactactactggaccctgctggagcccggcgacaccatcatcttcgaggccaccggcaacctgatcgccccctggtacgccttcgccctgagcagaggcttcgagagcggcatcatcaccagcaacgccagcatgcacgagtgcaacaccaagtgccagacccccatgggcgccatcaacagcagcatgcccttccacaacatccaccccctgaccatcggcgagtgccccaagtacgtgaagagcaacagactggtgctggccaccggcctgagaaacagcccccagagagagagaagaagaaagaagagaggcctgttcggcgccatcgccggcttcatcgagggcggctggcagggcatggtggacggctggtacggctaccaccacagcaacgagcagggcagcggctacgccgccgacaaggagagcacccagaaggccatcgacggcgtgaccaacaaggtgaacagcatcatcgacaagatgaacacccagttcgaggccgtgggcagagagttcaacaacctggagagaagaatcgagaacctgaacaagaagatggaggacggcttcctggacgtgtggacctacaacgccgagctgctggtgctgatggagaacgagagaaccctggacttccacgacagcaacgtgaagaacctgtacgacaaggtgagactgcagctgagagacaacgccaaggagctgggcaacggctgcttcgagttctaccacaagtgcgacaacgagtgcatggagagcgtgagaaacggcacctacgactacccccagtacagcgaggaggccagactgaagagagaggagatcagcggcgtgaagctggagagcatcggcatctaccagatcctgagcatctacagcaccgtggccagcagcctggccctggccatcatggtggccggcctgagcctgtggatgtgcagcaacggcagcctgcagtgcagaatctgcatc

SEQ ID NO: 16 - DNA, 1707 bp, Influenza virus
atgaaggccaagctgctggtgctgctgtacgccttcgtggccaccgacgccgacaccatctgcatcggctaccacgccaacaacagcaccgacaccgtggacaccatcttcgagaagaacgtggccgtgacccacagcgtgaacctgctggaggacagacacaacggcaagctgtgcaagctgaagggcaaggcccccctgcagctgggcaagtgcagcatcaccggctggctgctgggcaaccccgagtgcgacagcctgctgcccgccagaagctggagctacatcgtggagacccccaacagcgagaacggcgcctgctaccccggcgacttcatcgactacgaggagctgagagagcagctgagcagcgtgagcagcctggagagattcgagatcttccccaaggagagcagctggcccaaccacaccttcaacggcgtgaccgtgagctgcagccacagaggcaagagcagcttctacagaaacctgctgtggctgaccaaggagggcgacagctaccccaagctgaccaacagctacgtgaacaacaagggcaaggaggtgctggtgctgtggggcgtgcaccaccccagcagcagcgacgagcagcagagcctgtacagcaacggcaacgcctacgtgagcgtggccagcagcaactacaacagaagattcacccccgagatcgccgccagacccaaggtgaaggaccagcacggcagaatgaactactactggaccctgctggagcccggcgacaccatcatcttcgaggccaccggcaacctgatcgccccctggtacgccttcgccctgagcagaggcttcgagagcggcatcatcaccagcaacgccagcatgcacgagtgcaacaccaagtgccagaccccccagggcagcatcaacagcaacctgcccttccagaacatccaccccgtgaccatcggcgagtgccccaagtacgtgagaagcaccaagctgagaatggtgaccggcctgagaaacatccccagcatccagtacagaagaaagaagagaggcctgttcggcgccatcgccggcttcatcgagggcggctggaccggcatgatcgacggctggtacggctaccaccaccagaacgagcagggcagcggctacgccgccgaccagaagagcacccagaacgccatcaacggcatcaccaacaaggtgaacagcatcatcgagaagatgaacacccagttcaccgccgtgggcaaggagttcaacaacctggagaagagaatggagaacctgaacaagaaggtggacgacggcttcctggacatctggacctacaacgccgagctgctggtgctgctggagaacgagagaaccctggacttccacgacctgaacgtgaagaacctgtacgagaaggtgaagagccagctgaagaacaacgccaaggagatcggcaacggctgcttcgagttctaccacaagtgcgacaacgagtgcatggagagcgtgagaaacggcacctacgactaccccaagtacagcgaggagagcaagctgaacagagagaagatcgacggcgtgaagctggagagcatgggcgtgtaccagatcctggccatctacagcaccgtggccagcagcctggtgctgctggtgagcctgggcgccatcagcttctggatgtgcagcaacggcagcctgaagtgcagaatctgcatc

SEQ ID NO: 17 - DNA, 1704 bp, Influenza virus
atggagaagatcgtgctgctgttcgccatcgtgagcctggtgaagagcgaccagatctgcatcggctaccacgccaacaacagcaccgagcaggtggacaccatcatggagaagaacgtgaccgtgacccacgcccaggacatcctggagaagacccacaacggcaagctgtgcgacctggacggcgtgaagcccctgatcctgagagactgcagcgtggccggctggctgctgggcaaccccatgtgcgacgagttcatcaacgtgcccgagtggagctacatcgtggagaaggccaaccccgtgaacgacctgtgctaccccggcgacttcaacgactacgaggagctgaagcacctgctgagcagaatcaaccacttcgagaagatccagatcatccccaagagcagctggagcagccacgaggccagcctgggcgtgagcagcgcctgcccctaccagagaaagagcagcttcttcagaaacgtggtgtggctgatcaagaagaacagcacctaccccaccatcaagagaagctacaacaacaccaaccaggaggacctgctggtgctgtggggcatccaccaccccaacgacgccgccgagcagaccaagctgtaccagaaccccaccacctacatcagcgtgggcaccagcaccctgaaccagagactggtgcccagaatcgccaccagaagcaaggtgaacggccagagcggcagaatggagttcttctggaccatcctgaagcccaacgacgccatcaacttcgagagcaacggcaacttcatcgcccccgagtacgcctacaagatcgtgaagaagggcgacagcaccatcatgaagagcgagctggagtacggcaactgcaacaccaagtgccagacccccatgggcgccatcaacagcagcatgcccttccacaacatccaccccctgaccatcggcgagtgccccaagtacgtgaagagcaacagactggtgctggccaccggcctgagaaacagcccccagagagagagaagaagaaagaagagaggcctgttcggcgccatcgccggcttcatcgagggcggctggcagggcatggtggacggctggtacggctaccaccacagcaacgagcagggcagcggctacgccgccgacaaggagagcacccagaaggccatcgacggcgtgaccaacaaggtgaacagcatcatcgacaagatgaacacccagttcgaggccgtgggcagagagttcaacaacctggagagaagaatcgagaacctgaacaagaagatggaggacggcttcctggacgtgtggacctacaacgccgagctgctggtgctgatggagaacgagagaaccctggacttccacgacagcaacgtgaagaacctgtacgacaaggtgagactgcagctgagagacaacgccaaggagctgggcaacggctgcttcgagttctaccacaagtgcgacaacgagtgcatggagagcgtgagaaacggcacctacgactacccccagtacagcgaggaggccagactgaagagagaggagatcagcggcgtgaagctggagagcatcggcatctaccagatcctgagcatctacagcaccgtggccagcagcctggccctggccatcatggtggccggcctgagcctgtggatgtgcagcaacggcagcctgcagtgcagaatctgcatc

SEQ ID NO: 18 - DNA, 1698 bp, Influenza virus
atggccatcatctacctgatcctgctgttcaccgccgtgagaggcgaccagatctgcatcggctaccacgccaacaacagcaccgagaaggtggacaccatcctggagagaaacgtgaccgtgacccacgccaaggacatcctggagaagacccacaacggcaagctgtgcaagctgaacggcatcccccccctggagctgggcgactgcagcatcgccggctggctgctgggcaaccccgagtgcgacagactgctgagcgtgcccgagtggagctacatcatggagaaggagaaccccagagacggcctgtgctaccccggcagcttcaacgactacgaggagctgaagcacctgctgagcagcgtgaagcacttcgagaaggtgaagatcctgcccaaggacagatggacccagcacaccaccaccggcggcagcagagcctgcgccgtgagcggcaaccccagcttcttcagaaacatggtgtggctgaccaagaagggcagcgactaccccgtggccaagggcagctacaacaacaccagcggcgagcagatgctgatcatctggggcgtgcaccaccccaacgacgagaccgagcagagaaccctgtaccagaacgtgggcacctacgtgagcgtgagcaccagcaccctgaacaagagaagcacccccgagatcgccaccagacccaaggtgaacggcctgggcagcagaatggagttcagctggaccctgctggacatgtgggacaccatcaacttcgagagcaccggcaacctgatcgcccccgagtacggcttcaagatcagcaagagaggcagcagcggcatcatgaagaccgagggcaccctggagaactgcgagaccaagtgccagacccccctgggcgccatcaacaccaccctgcccttccacaacgtgcaccccctgaccatcggcgagtgccccaagtacgtgaagagcgagaagctggtgctggccaccggcctgagaaacgtgccccagatcgagagcagaagaaagaagagaggcctgttcggcgccatcgccggcttcatcgagggcggctggcagggcatggtggacggctggtacggctaccaccacagcaacgaccagggcagcggctacgccgccgacaaggagagcacccagaaggccttcgacggcatcaccaacaaggtgaacagcgtgatcgagaagatgaacacccagttcgaggccgtgggcaaggagttcggcaacctggagagaagactggagaacctgaacaagaagatggaggacggcttcctggacgtgtggacctacaacgccgagctgctggtgctgatggagaacgagagaaccctggacttccacgacagcaacgtgaagaacctgtacgacaaggtgagaatgcagctgagagacaacgtgaaggagctgggcaacggctgcttcgagttctaccacaagtgcgacgacgagtgcatgaacagcgtgaagaacggcacctacgactaccccaagtacgaggaggagagcaagctgaacagaaacgagatcaagggcgtgaagctgagcagcatgggcgtgtaccagatcctggccatctacgccaccgtggccggcagcctgagcctggccatcatgatggccggcatcagcttctggatgtgcagcaacggcagcctgcagtgcagaatctgcatc

SEQ ID NO: 19 - DNA, 1704 bp, Influenza virus
atggagaagatcgtgctgctgttcgccgtggtgagcctggtgaagagcgaccagatctgcatcggctaccacgccaacaacagcaccgagcaggtggacaccatcatggagaagaacgtgaccgtgacccacgcccaggacatcctggagagaacccacaacggcaagctgtgcgacctggacggcgtgaagcccctgatcctgagagactgcagcgtggccggctggctgctgggcaaccccatgtgcgacgagttcatcaacgtgcccgagtggagctacatcgtggagagagccaaccccgtgaacggcctgtgctaccccggcgacttcaacgactacgaggagctgaagcacctgctgagcagaatcaaccacttcgagaagatccagatcatccccaagagcagctggagcagccacgaggccagcctgggcgtgagcagcgcctgcccctacctgggcaagcccagcttcttcagaaacgtggtgtggctgatcaagaagaacagcacctaccccaccatcaagagaagctacaacaacaccaaccaggaggacctgctggtgatgtggggcatccaccaccccaacgacgccgccgagcagaccaagctgtaccagaaccccaccacctacatcagcgtgggcaccagcaccctgaaccagagactggtgcccagaatcgccaccagaagcaaggtgaacggccagagcggcagaatggagttcttctggaccatcctgaagcccaacgacgccatcaacttcgagagcaacggcaacttcatcgcccccgagtacgcctacaagatcgtgaagaagggcgacagcaccatcatgaagagcgagctggagtacggcaactgcaacaccaagtgccagacccccatgggcgccatcaacagcagcatgcccttccacaacatccaccccctgaccatcggcgagtgccccaagtacgtgaagagcaacagactggtgctggccaccggcctgagaaacagcccccagagagagagaagaagaaagaagagaggcctgttcggcgccatcgccggcttcatcgagggcggctggcagggcatggtggacggctggtacggctaccaccacagcaacgagcagggcagcggctacgccgccgacaaggagagcacccagaaggccatcgacggcgtgaccaacaaggtgaacagcatcatcgacaagatgaacacccagttcgaggccgtgggcagagagttcaacaacctggagagaagaatcgagaacctgaacaagaagatggaggacggcttcctggacgtgtggacctacaacgccgagctgctggtgctgatgggcaacgagagaaccctggacttccacgacagcaacgtgaagaacctgtacgacaaggtgagactgcagctgagagacaacgccaaggagctgggcaacggctgcttcgagttctaccacaagtgcgacaacgagtgcatggagagcgtgagaaacggcacctacgactacccccagtacagcgaggaggccagactgaagagagaggagatcagcggcgtgaagctggagagcatcggcatctaccagatcctgagcatctacagcaccgtggccagcagcctggccctggccatcatgatcgccggcctgagcctgtggatgtgcagcaacggcagcctgcagtgcagaatctgcatc

SEQ ID NO: 20 - DNA, 1701 bp, Influenza virus
atggagaagatcgtgctgctgttcgccatcgtgagcctggtgaagagcgaccagatctgcatcggctaccacgccaacaacagcaccgagcaggtggacaccatcatggagaagaacgtgaccgtgacccacgcccaggacatcctggagaagacccacaacggcaagctgtgcgacctggacggcgtgaagcccctgatcctgagagactgcagcgtggccggctggctgctgggcaaccccatgtgcgacgagttcatcaacgtgcccgagtggagctacatcgtggagaaggccaaccccgtgaacgacctgtgctaccccggcgacttcaacgactacgaggagctgaagcacctgctgagcagaatcaaccacttcgagaagatccagatcatccccaagagcagctggagcagccacgaggccagcctgggcgtgagcgccgcctgcccctaccagggcaagagcagcttcttcagaaacgtggtgtggctgatcaagaagaacagcacctaccccaccatcaagagaagctacaacaacaccaaccaggaggacctgctggtgatgtggggcatccaccaccccaacgacgaggccgagcagaccaagctgtaccagaaccccatcacctacatcagcgtgggcaccagcaccctgaaccagagactggtgcccagaatcgccaccagaagcaaggtgaacggccagagcggcagaatggagttcttctggaccatcctgaagcccaacgacgccatcaacttcgagagcaacggcaacttcatcgcccccgagtacgcctacaagatcgtgaagaagggcgacagcaccatcatgaagagcgagctggagtacggcaactgcaacaccaagtgccagacccccatgggcgccatcaacagcagcatgcccttccacaacatccaccccctgaccatcggcgagtgccccaagtacgtgaagagcaacagactggtgctggccaccggcctgagaaacagcccccagagagagagaagaaagaagagaggcctgttcggcgccatcgccggcttcatcgagggcggctggcagggcatggtggacggctggtacggctaccaccacagcaacgagcagggcagcggctacgccgccgacaaggagagcacccagaaggccatcgacggcgtgaccaacaaggtgaacagcatcatcgacaagatgaacacccagttcgaggccgtgggcagagagttcaacaacctggagagaagaatcgagaacctgaacaagaagatggaggacggcttcctggacgtgtggacctacaacgccgagctgctggtgctgatggagaacgagagaaccctggacttccacgacagcaacgtgaagaacctgtacgacaaggtgagactgcagctgagagacaacgccaaggagctgggcaacggctgcttcgagttctaccacaagtgcgacaacgagtgcatggagagcgtgagaaacggcacctacgactacccccagtacagcgaggaggccagactgaagagagaggagatcagcggcgtgaagctggagagcatcggcatctaccagatcctgagcatctacagcaccgtggccagcagcctggccctggccatcatggtggccggcctgagcctgtggatgtgcagcaacggcagcctgcagtgcagaatctgcatc

SEQ ID NO: 21 - DNA, 1701 bp, Influenza virus
atggagaagatcgtgctgctgctggccatcgtgagcctggtgaagagcgaccagatctgcatcggctaccacgccaacaacagcaccgagcaggtggacaccatcatggagaagaacgtgaccgtgacccacgcccaggacatcctggagaagacccacaacggcaagctgtgcgacctggacggcgtgaagcccctgatcctgagagactgcagcgtggccggctggctgctgggcaaccccatgtgcgacgagttcatcaacgtgcccgagtggagctacatcgtggagaaggccaaccccgccaacgacctgtgctaccccggcaacttcaacgactacgaggagctgaagcacctgctgagcagaatcaaccacttcgagaagatccccatcatccccaagagcagctggagcgaccacgaggccagcagcggcgtgagcagcgcctgcccctaccagggcacccccagcttcttcagaaacgtggtgtggctgatcaagaagaacaacacctaccccaccatcaagagaagctacaacaacaccaaccaggaggacctgctgatcctgtggggcatccaccacagcaacaacgccgccgagcagaccaagctgtaccagaaccccaccacctacatcagcgtgggcaccagcaccctgaacctgagactggtgcccaagatcgccaccagaagcaaggtgaacggccagagcggcagaatggacttcttctggaccatcctgaagcccaacgacgccatcaacttcgagagcaacggcaacttcatcgcccccgagtacgcctacaagatcgtgaagaagggcgacagcgccatcatgaagagcgaggtggagtacggcaactgcaacaccaagtgccagacccccatcggcgccatcaacagcagcatgcccttccacaacatccaccccctgaccatcggcgagtgccccaagtacgtgaagagcaacaagctggtgctggccaccggcctgagaaacagccccctgagagagagaagaagaaagagaggcctgttcggcgccatcgccggcttcatcgagggcggctggcagggcatggtggacggctggtacggctaccaccacagcaacgagcagggcagcggctacgccgccgacaaggagagcacccagaaggccatcgacggcgtgaccaacaaggtgaacagcatcatcgacaagatgaacacccagttcgaggccgtgggcagagagttcaacaacctggagagaagaatcgagaacctgaacaagaagatggaggacggcttcctggacgtgtggacctacaacgccgagctgctggtgctgatggagaacgagagaaccctggacttccacgacagcaacgtgaagaacctgtacgacaaggtgagactgcagctgagagacaacgccaaggagctgggcaacggctgcttcgagttctaccacaagtgcgacaacgagtgcatggagagcgtgagaaacggcacctacgactacccccagtacagcgaggaggccagactgaagagagaggagatcagcggcgtgaagctggagagcatcggcacctaccagatcctgagcatctacagcaccgtggccagcagcctggccctggccatcatggtggccggcctgagcctgtggatgtgcagcaacggcagcctgcagtgcagaatctgcatc

SEQ ID NO: 22 - DNA, 1347 bp, Influenza virus
atgaaccccaacaagaagatcatcaccatcggcagcatctgcatggtgaccggcatggtgagcctgatgctgcagatcggcaacctgatcagcatctgggtgagccacagcatccacaccggcaaccagcacaaggccgagcccatcagcaacaccaacttcctgaccgagaaggccgtggccagcgtgaagctggccggcaacagcagcctgtgccccatcaacggctgggccgtgtacagcaaggacaacagcatcagaatcggcagcaagggcgacgtgttcgtgatcagagagcccttcatcagctgcagccacctggagtgcagaaccttcttcctgacccagggcgccctgctgaacgacaagcacagcaacggcaccgtgaaggacagaagcccccacagaaccctgatgagctgccccgtgggcgaggcccccagcccctacaacagcagattcgagagcgtggcctggagcgccagcgcctgccacgacggcaccagctggctgaccatcggcatcagcggccccgacaacggcgccgtggccgtgctgaagtacaacggcatcatcaccgacaccatcaagagctggagaaacaacatcctgagaacccaggagagcgagtgcgcctgcgtgaacggcagctgcttcaccgtgatgaccgacggccccagcaacggccaggccagccacaagatcttcaagatggagaagggcaaggtggtgaagagcgtggagctggacgcccccaactaccactacgaggagtgcagctgctaccccgacgccggcgagatcacctgcgtgtgcagagacaactggcacggcagcaacagaccctgggtgagcttcaaccagaacctggagtaccagatcggctacatctgcagcggcgtgttcggcgacaaccccagacccaacgacggcaccggcagctgcggccccgtgagcagcaacggcgcctacggcgtgaagggcttcagcttcaagtacggcaacggcgtgtggatcggcagaaccaagagcaccaacagcagaagcggcttcgagatgatctgggaccccaacggctggaccgagaccgacagcagcttcagcgtgaagcaggacatcgtggccatcaccgactggagcggctacagcggcagcttcgtgcagcaccccgagctgaccggcctggactgcatcagaccctgcttctgggtggagctgatcagaggcagacccaaggagagcaccatctggaccagcggcagcagcatcagcttctgcggcgtgaacagcgacaccgtgggctggagctggcccgacggcgccgagctgcccttcaccatcgacaag

SEQ ID NO: 23 - DNA, 1347 bp, Influenza virus
atgaaccccaaccagaagatcatcaccatcggcagcatctgcatgatcaccggcatggtgagcctgatgctgcaggtgggcaacatgatcagcatctggatcagccacagcatccacaccggcaaccagcaccaggccgagcccatcagcaacgccaacttcctgaccaagagagccgtggccgccgtgaagctggccggcaacagcagcctgtgccccatcaacggctgggccgtgtacagcaaggacaacagcatcagaatcggcagcaagggcgacgtgttcgtgatcagagagcccttcatcagctgcagccacctggagtgcagaaccttcttcctgacccagggcgccctgctgaacgacaagcacagcaacggcaccgccaaggacagaagcccccacagaaccctgatgagctgccccgtgggcgaggcccccagcccctacaacagcagattcgagagcgtggcctggagcgccagcgcctgccacgacggcaccagctggctgaccatcggcatcagcggccccgacaacggcgccgtggccgtgctgaagtacaacggcatcatcaccgacaccatcaagagctggagaaacaacatcctgagaacccaggagagcgagtgcgcctgcgtgaacggcagctgcttcaccgtgatgaccgacggccccagcaacggccaggccagccacaagatcttcaagatggagaagggcaaggtggtgaagagcgtggagctggacgcccccaactaccactacgaggagtgcagctgctaccccgacgccggcgagatcacctgcgtgtgcagagacaactggcacggcagcaacagaccctgggtgagcttcaaccagaacctggagtaccagatcggctacatctgcagcggcgtgttcggcgacaaccccagacccaacgacggcaccggcagctgcggccccgtgagcagcaacggcgcctacggcgtgaagggcttcagcttcaagtacggcaacggcgtgtggatcggcagaaccaagagcaccaacagcagaagcggcttcgagatgatctgggaccccaacggctggaccgagaccgacagcagcttcagcgtgaagcaggacatcgtggccatcaccgactggagcggctacagcggcagcttcgtgcagcaccccgagctgaccggcctggactgcatcagaccctgcttctggatcgagctgatcagaggcagacccaaggagagcaccatctggaccagcggcagcagcatcagcttctgcggcgtgaacagcgacaccgtgggctggagctggcccgacggcgccgagctgcccttcaccatcgacaag

SEQ ID NO: 24 - DNA, 1347 bp, Influenza virus
atgaaccccaaccagaagatcatcaccatcggcagcatctgcatggtgatcggcatcgtgagcctgatgctgcagatcggcaacatggtgagcctgtgggtgagccacagcatccagaccggcaaccagcaccaggtggagcccatcagcaacaccaacttcctgaccgagaaggccgtggccagcgtgaccctggccggcaacagcagcctgtgccccatcagaggctgggccgtgcacagcaaggacaacagcatcagaatcggcagcaagggcgacgtgttcgtgatcagagagcccttcatcagctgcagccacctggagtgcagaaccttcttcctgacccagggcgccctgctgaacgacaagcacagcaacggcaccgtgaaggacagaagcccccacagaaccctgatgagctgccccgtgggcgaggcccccagcccctacaacagcagattcgagagcgtggcctggagcgccagcgcctgccacgacggcaccagctggctgaccatcggcatcagcggccccgacaacggcgccgtggccgtgctgaagtacaacggcatgatcaccgacaccatcaagagctggagaaacaacatcctgagaacccaggagagcgagtgcgcctgcgtgaacggcagctgcttcaccgtgatgaccgacggccccagcaacggccaggccagctacaagatcttcaagatggagaagggcaaggtggtgaagagcgtggagctggacgcccccaactaccactacgaggagtgcagctgctaccccgacgccggcgagatcacctgcgtgtgcagagacaactggcacggcagcaacagaccctgggtgagcttcaaccagaacctggagtaccagatcggctacatctgcagcggcgtgttcggcgacaaccccagacccaacggcggcaccggcagctgcggccccgtgagccccaacggcgcctacggcgtgaagggcttcagcttcaagtacggcaacggcgtgtggatcggcagaaccaagagccccagcagcagaagcggcttcgagatgatctgggaccccaacggctggaccgagaccgacagcagcttcagcgtgaagcaggacatcgtggccatcaccgactggagcggctacagcggcagcttcgtgcagcaccccgagctgaccggcctggactgcatcagaccctgcttctgggtggagctgatcagaggcagacccaaggagagcaccatctggaccagcggcagcagcatcagcttctgcggcgtgaacagcgacaccgtgagctggagctggcccgacggcgccgagctgcccttcaccatcgacaag

SEQ ID NO: 25 - DNA, 1347 bp, Influenza virus
atgaaccccaaccagaagatcatcaccatcggcagcatctgcatggtgatcggcatcgtgagcctgatgctgcagatcggcaacatgatcagcatctgggtgagccacagcatccagaccggcaaccagcaccaggccgagcccatcagcaacaccaacttcctgaccgagaacgccgtggccagcgtgaccctggccggcaacagcagcctgtgccccatcagaggctgggccgtgcacagcaaggacaacagcatcagaatcggcagcaagggcgacgtgttcgtgatcagagagcccttcatcagctgcagccacctggagtgcagaaccttcttcctgacccagggcgccctgctgaacgacaagcacagcaacggcaccgtgaaggacagaagcccccacggcaccctgatgagctgccccatgggcgaggcccccagcccctacaacagcagattcgagagcgtggcctggagcgccagcgcctgccacgacggcaccagctggctgaccatcggcatcagcggccccgacaacggcgccgtggccgtgctgaagtacaacggcatcatcaccgacaccatcaagagctggagaaacaacatcctgagaacccaggagagcgagtgcgcctgcgtgaacggcagctgcttcaccgtgatgaccgacggccccagcaacggccaggccagctacaagatcttcaagatggagaagggcaaggtggtgaagagcgtggagctgaacgcccccaactaccactacgaggagtgcagctgctaccccgacgccggcgagatcatctgcgtgtgcagagacaactggcacggcagcaacagaccctgggtgagcttcaaccagaacctggagtaccagatcggctacatctgcagcggcgtgttcggcgacaaccccagacccaacgacggcaccggcagctgcggccccgtgagccccaacggcgcctacggcatcaagggcttcagcttcaagtacggcaacggcgtgtggatcggcagaaccaagagcaccaacagcagaagcggcttcgagatgatctgggaccccaacggctggaccggcaccgacagcgacttcagcgtgaagcaggacatcgtggccatcaccgactggagcggctacagcggcagcttcgtgcagcaccccgagctgaccggcctggactgcatcagaccctgcttctgggtggagctgatcagaggcagacccaaggagagcaccatctggaccagcggcagcagcatcagcttctgcggcgtgaacagcgacaccgtgagctggagctggcccgacggcgccgagctgcccttcaccatcgacaag

SEQ ID NO: 26 - protein, 457 aa, Artificial
  H5N1 NAisolate A/Thailand/1(KAN-1)/04 + FLAG epitope at residue 50
MNPNKKIITIGSICMVTGMVSLMLQIGNLISIWVSHSIHTGNQHKAEPISDYKDDDDKNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPDAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 27 - protein, 567 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIVKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 28 - protein, 568 aa, Influenza virus; identical to SEQ ID NO: 5

SEQ ID NO: 29 - protein, 567 aa, Influenza virus; identical to SEQ ID NO: 6

SEQ ID NO: 30 - protein, 533 aa, Influenza virus
MEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 31 - protein, 568 aa, Influenza virus
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 32 - protein, 567 aa, Influenza virus
MEKIVFLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVIWLIKKNNTYPTIKRSYNNTNQEDLLILWGIHHSNDAAEQTKLYQNPTTYISVGTSTLNQRWVPKIATRSKVNGQSGRMDFFWTILKPNDAINFESNGNFIAPEYAYKIVKKGDSAILKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAIGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 33 - protein, 568 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSEVNGQSGRMEFFWTILKPNDAINFESNGNFVAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAANKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 34 - protein, 568 aa, Influenza virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 35 - protein, 569 aa, Influenza virus
MKAKLLVLLYAFVATDADTICIGYHANNSTDTVDTIFEKNVAVTHSVNLLEDRHNGKLCKLKGIAPLQLGKCNITGWLLGNPECDSLLPARSWSYIVETPNSENGACYPGDFIDYEELREQLSSVSSLERFEIFPKESSWPNHTFNGVTVSCSHRGKSSFYRNLLWLTKKGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRRKKRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 36 - DNA, 1371 bp, Artificial
  H5N1 Thailand 04 + Flag coding sequence
atgaacccgaacaaaaaaattattaccattggcagcatttgcatggtgaccggcatggtgagcctgatgctgcagattggcaacctgattagcatttgggtgagccatagcattcataccggcaaccagcataaagcggaaccgattagcgattataaagatgatgatgataaaaacaccaactttctgaccgaaaaagcggtggcgagcgtgaaactggcgggcaacagcagcctgtgcccgattaacggctgggcggtgtatagcaaagataacagcattcgcattggcagcaaaggcgatgtgtttgtgattcgcgaaccgtttattagctgcagccatctggaatgccgcaccttttttctgacccagggcgcgctgctgaacgataaacatagcaacggcaccgtgaaagatcgcagcccgcatcgcaccctgatgagctgcccggtgggcgaagcgccgagcccgtataacagccgctttgaaagcgtggcgtggagcgcgagcgcgtgccatgatggcaccagctggctgaccattggcattagcggcccggataacggcgcggtggcggtgctgaaatataacggcattattaccgataccattaaaagctggcgcaacaacattctgcgcacccaggaaagcgaatgcgcgtgcgtgaacggcagctgctttaccgtgatgaccgatggcccgagcaacggccaggcgagccataaaatttttaaaatggaaaaaggcaaagtggtgaaaagcgtggaactggatgcgccgaactatcattatgaagaatgcagctgctatccggatgcgggcgaaattacctgcgtgtgccgcgataactggcatggcagcaaccgcccgtgggtgagctttaaccagaacctggaatatcagattggctatatttgcagcggcgtgtttggcgataacccgcgcccgaacgatggcaccggcagctgcggcccggtgagcagcaacggcgcgtatggcgtgaaaggctttagctttaaatatggcaacggcgtgtggattggccgcaccaaaagcaccaacagccgcagcggctttgaaatgatttgggatccgaacggctggaccgaaaccgatagcagctttagcgtgaaacaggatattgtggcgattaccgattggagcggctatagcggcagctttgtgcagcatccggaactgaccggcctggattgcattcgcccgtgcttttgggtggaactgattcgcggccgcccgaaagaaagcaccatttggaccagcggcagcagcattagcttttgcggcgtgaacagcgataccgtgggctggagctggccggatggcgcggaactgccgtttaccattgataaa

SEQ ID NO: 37 - DNA, 1701 bp, Influenza virus
atggaaaaaattgtgctgctgctggcgattgtgagcctggtgaaaagcgatcagatttgcattggctatcatgcgaacaacagcaccgaacaggtggataccattatggaaaaaaacgtgaccgtgacccatgcgcaggatattctggaaaaaacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccggcgaacgatctgtgctatccgggcaactttaacgattatgaagaactgaaacatctgctgagccgcattaaccattttgaaaaaattcagattattccgaaaagcagctggagcgatcatgaagcgagcagcggcgtgagcagcgcgtgcccgtatcagggcaccccgagcttttttcgcaacgtggtgtggctgattaaaaaaaacaacacctatccgaccattaaacgcagctataacaacaccaaccaggaagatctgctgattctgtggggcattcatcatagcaacgatgcggcggaacagaccaaactgtatcagaacccgaccacctatattagcgtgggcaccagcaccctgaaccagcgcctggtgccgaaaattgcgacccgcagcaaagtgaacggccagagcggccgcatggattttttttggaccattctgaaaccgaacgatgcgattaactttgaaagcaacggcaactttattgcgccggaatatgcgtataaaattgtgaaaaaaggcgatagcgcgattgtgaaaagcgaagtggaatatggcaactgcaacaccaaatgccagaccccgattggcgcgattaacagcagcatgccgtttcataacattcatccgctgaccattggcgaatgcccgaaatatgtgaaaagcaacaaactggtgctggcgaccggcctgcgcaacagcccgctgcgcgaacgccgccgcaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcacctatcagattctgagcatttatagcaccgtggcgagcagcctggcgctggcgattatggtggcgggcctgagcctgtggatgtgcagcaacggcagcctgcagtgccgcatttgcatt

SEQ ID NO: 38 - DNA, 1704 bp, Influenza virus
atggaaaaaattgtgctgctgtttgcggtggtgagcctggtgaaaagcgatcagatttgcattggctatcatgcgaacaacagcaccgaacaggtggataccattatggaaaaaaacgtgaccgtgacccatgcgcaggatattctggaacgcacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaacgcgcgaacccggtgaacggcctgtgctatccgggcgattttaacgattatgaagaactgaaacatctgctgagccgcattaaccattttgaaaaaattcagattattccgaaaagcagctggagcagccatgaagcgagcctgggcgtgagcagcgcgtgcccgtatctgggcaaaccgagcttttttcgcaacgtggtgtggctgattaaaaaaaacagcacctatccgaccattaaacgcagctataacaacaccaaccaggaagatctgctggtgatgtggggcattcatcatccgaacgatgcggcggaacagaccaaactgtatcagaacccgaccacctatattagcgtgggcaccagcaccctgaaccagcgcctggtgccgcgcattgcgacccgcagcaaagtgaacggccagagcggccgcatggaatttttttggaccattctgaaaccgaacgatgcgattaactttgaaagcaacggcaactttattgcgccggaatatgcgtataaaattgtgaaaaaaggcgatagcaccattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacagcccgcagcgcgaacgccgccgcaaaaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatgggcaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcatttatcagattctgagcatttatagcaccgtggcgagcagcctggcgctggcgattatgattgcgggcctgagcctgtggatgtgcagcaacggcagcctgcagtgccgcatttgcatt

SEQ ID NO: 39 - DNA, 1701 bp, Influenza virus
atggaaaaaattgtgctgctgtttgcgattgtgagcctggtgaaaagcgatcagatttgcattggctatcatgcgaacaacagcaccgaacaggtggataccattatggaaaaaaacgtgaccgtgacccatgcgcaggatattctggaaaaaacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccggtgaacgatctgtgctatccgggcgattttaacgattatgaagaactgaaacatctgctgagccgcattaaccattttgaaaaaattcagattattccgaaaagcagctggagcagccatgaagcgagcctgggcgtgagcgcggcgtgcccgtatcagggcaaaagcagcttttttcgcaacgtggtgtggctgattaaaaaaaacagcacctatccgaccattaaacgcagctataacaacaccaaccaggaagatctgctggtgatgtggggcattcatcatccgaacgatgaagcggaacagaccaaactgtatcagaacccgattacctatattagcgtgggcaccagcaccctgaaccagcgcctggtgccgcgcattgcgacccgcagcaaagtgaacggccagagcggccgcatggaatttttttggaccattctgaaaccgaacgatgcgattaactttgaaagcaacggcaactttattgcgccggaatatgcgtataaaattgtgaaaaaaggcgatagcaccattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacagcccgcagcgcgaacgccgcaaaaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcatttatcagattctgagcatttatagcaccgtggcgagcagcctggcgctggcgattatggtggcgggcctgagcctgtggatgtgcagcaacggcagcctgcagtgccgcatttgcatt

SEQ ID NO: 40 - DNA, 1599 bp, Influenza virus
atggaaaaaaacgtgaccgtgacccatgcgcaggatattctggaacgcacccataacggcaaactgtgcgatctgaacggcgtgaaaccgctgattctgcgcgattgcagcgtggcgggctggctgctgggcaacccgatgtgcgatgaatttattaacgtgccggaatggagctatattgtggaaaaagcgagcccggcgaacgatctgtgctatccgggcaactttaacgattatgaagaactgaaacatctgctgagccgcattaaccattttgaaaaaattcagattattccgaaaagcagctggagcaaccatgatgcgagcagcggcgtgagcagcgcgtgcccgtatctgggccgcagcagcttttttcgcaacgtggtgtggctgattaaaaaaaacagcgcgtatccgaccattaaacgcagctataacaacaccaaccaggaagatctgctggtgctgtggggcattcatcatccgaacgatgcggcggaacagaccaaactgtatcagaacccgaccacctatattagcgtgggcaccagcaccctgaaccagcgcctggtgccggaaattgcgacccgcccgaaagtgaacggccagagcggccgcatggaatttttttggaccattctgaaaccgaacgatgcgattaactttgaaagcaacggcaactttattgcgccggaatatgcgtataaaattgtgaaaaaaggcgatagcaccattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacaccccgcagcgcgaacgccgccgcaaaaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattaacaaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgaaaaacggcacctatgattatccgcagtatagcgaagaagcgcgcctgaaccgcgaagaaattagcggcgtgaaactggaaagcatgggcacctatcagattctgagcatttatagcaccgtggcgagcagcctggcgctggcgattatggtggcgggcctgagcctgtggatgtgcagcaacggcagcctgcagtgccgcatttgcatt

SEQ ID NO: 41 - DNA, 1704 bp, Influenza virus
atggaaaaaattgtgctgctgctggcgattgtgagcctggtgaaaagcgatcagatttgcattggctatcatgcgaacaacagcaccgaacaggtggataccattatggaaaaaaacgtgaccgtgacccatgcgcaggatattctggaaaaaacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccgaccaacgatctgtgctatccgggcagctttaacgattatgaagaactgaaacatctgctgagccgcattaaccattttgaaaaaattcagattattccgaaaagcagctggagcgatcatgaagcgagcagcggcgtgagcagcgcgtgcccgtatctgggcagcccgagcttttttcgcaacgtggtgtggctgattaaaaaaaacagcacctatccgaccattaaaaaaagctataacaacaccaaccaggaagatctgctggtgctgtggggcattcatcatccgaacgatgcggcggaacagacccgcctgtatcagaacccgaccacctatattagcattggcaccagcaccctgaaccagcgcctggtgccgaaaattgcgacccgcagcaaagtgaacggccagagcggccgcatggaatttttttggaccattctgaaaccgaacgatgcgattaactttgaaagcaacggcaactttattgcgccggaatatgcgtataaaattgtgaaaaaaggcgatagcgcgattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacagcccgcagcgcgaaagccgccgcaaaaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcattcgcaacggcacctataactatccgcagtatagcgaagaagcgcgcctgaaacgcgaagaaattagcggcgtgaaactggaaagcattggcacctatcagattctgagcatttatagcaccgtggcgagcagcctggcgctggcgattatgatggcgggcctgagcctgtggatgtgcagcaacggcagcctgcagtgccgcatttgcatt

SEQ ID NO: 42 - DNA, 1701 bp, Influenza virus
atggaaaaaattgtgtttctgctggcgattgtgagcctggtgaaaagcgatcagatttgcattggctatcatgcgaacaacagcaccgaacaggtggataccattatggaaaaaaacgtgaccgtgacccatgcgcaggatattctggaaaaaacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccggcgaacgatctgtgctatccgggcaactttaacgattatgaagaactgaaacatctgctgagccgcattaaccattttgaaaaaattcagattattccgaaaagcagctggagcgatcatgaagcgagcagcggcgtgagcagcgcgtgcccgtatcagggcaccccgagcttttttcgcaacgtgatttggctgattaaaaaaaacaacacctatccgaccattaaacgcagctataacaacaccaaccaggaagatctgctgattctgtggggcattcatcatagcaacgatgcggcggaacagaccaaactgtatcagaacccgaccacctatattagcgtgggcaccagcaccctgaaccagcgctgggtgccgaaaattgcgacccgcagcaaagtgaacggccagagcggccgcatggattttttttggaccattctgaaaccgaacgatgcgattaactttgaaagcaacggcaactttattgcgccggaatatgcgtataaaattgtgaaaaaaggcgatagcgcgattctgaaaagcgaagtggaatatggcaactgcaacaccaaatgccagaccccgattggcgcgattaacagcagcatgccgtttcataacattcatccgctgaccattggcgaatgcccgaaatatgtgaaaagcaacaaactggtgctggcgaccggcctgcgcaacagcccgctgcgcgaacgccgccgcaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcgattggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcacctatcagattctgagcatttatagcaccgtggcgagcagcctggcgctggcgattatggtggcgggcctgagcctgtggatgtgcagcaacggcagcctgcagtgccgcatttgcatt

SEQ ID NO: 43 - DNA, 1704 bp, Influenza virus
atggaaaaaattgtgctgctgtttgcgattgtgagcctggtgaaaagcgatcagatttgcattggctatcatgcgaacaacagcaccgaacaggtggataccattatggaaaaaaacgtgaccgtgacccatgcgcaggatattctggaaaaaacccataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccggtgaacgatctgtgctatccgggcgattttaacgattatgaagaactgaaacatctgctgagccgcattaaccattttgaaaaaattcagattattccgaaaagcagctggagcagccatgaagcgagcctgggcgtgagcagcgcgtgcccgtatcagggcaaaagcagcttttttcgcaacgtggtgtggctgattaaaaaaaacagcacctatccgaccattaaacgcagctataacaacaccaaccaggaagatctgctggtgctgtggggcattcatcatccgaacgatgcggcggaacagaccaaactgtatcagaacccgaccacctatattagcgtgggcaccagcaccctgaaccagcgcctggtgccgcgcattgcgacccgcagcgaagtgaacggccagagcggccgcatggaatttttttggaccattctgaaaccgaacgatgcgattaactttgaaagcaacggcaactttgtggcgccggaatatgcgtataaaattgtgaaaaaaggcgatagcaccattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacagcccgcagcgcgaacgccgccgcaaaaaacgcggcctgtttggcgcgattgcgggctttattgaaggcggctggcagggcatggtggatggctggtatggctatcatcatagcaacgaacagggcagcggctatgcggcgaacaaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcatttatcagattctgagcatttatagcaccgtggcgagcagcctggcgctggcgattatggtggcgggcctgagcctgtggatgtgcagcaacggcagcctgcagtgccgcatttgcatt

SEQ ID NO: 44 - DNA, 1704 bp, Influenza virus
atggaaaaaattgtgctgctgtttgcgattgtgagcctggtgaaaagcgatcagatttgcattggctatcatgcgaacaacagcaccgaacaggtggataccattatggaaaaaaacgtgaccgtgacccatgcgcaggatattctggaaaaaaaacataacggcaaactgtgcgatctggatggcgtgaaaccgctgattctgcgcgattgcagcgtggcgggctggctgctgggcaacccgatgtgcgatgaatttattaacgtgccggaatggagctatattgtggaaaaagcgaacccggtgaacgatctgtgctatccgggcgattttaacgattatgaagaactgaaacatctgctgagccgcattaaccattttgaaaaaattcagattattccgaaaagcagctggagcagccatgaagcgagcctgggcgtgagcagcgcgtgcccgtatcagggcaaaagcagcttttttcgcaacgtggtgtggctgattaaaaaaaacagcacctatccgaccattaaacgcagctataacaacaccaaccaggaagatctgctggtgctgtggggcattcatcatccgaacgatgcggcggaacagaccaaactgtatcagaacccgaccacctatattagcgtgggcaccagcaccctgaaccagcgcctggtgccgcgcattgcgacccgcagcaaagtgaacggccagagcggccgcatggaatttttttggaccattctgaaaccgaacgatgcgattaactttgaaagcaacggcaactttattgcgccggaatatgcgtataaaattgtgaaaaaaggcgatagcaccattatgaaaagcgaactggaatatggcaactgcaacaccaaatgccagaccccgatgggcgcgattaacagcagcatgccgtttcataacattcatccgctgaccattggcgaatgcccgaaatatgtgaaaagcaaccgcctggtgctggcgaccggcctgcgcaacagcccgcagcgcgaacgccgccgcaaaaaacgcggcctgtttggcgcgattgcgggctttattgaaggcggctggcagggcatggtggatggctggtatggctatcatcatagcaacgaacagggcagcggctatgcggcggataaagaaagcacccagaaagcgattgatggcgtgaccaacaaagtgaacagcattattgataaaatgaacacccagtttgaagcggtgggccgcgaatttaacaacctggaacgccgcattgaaaacctgaacaaaaaaatggaagatggctttctggatgtgtggacctataacgcggaactgctggtgctgatggaaaacgaacgcaccctggattttcatgatagcaacgtgaaaaacctgtatgataaagtgcgcctgcagctgcgcgataacgcgaaagaactgggcaacggctgctttgaattttatcataaatgcgataacgaatgcatggaaagcgtgcgcaacggcacctatgattatccgcagtatagcgaagaagcgcgcctgaaacgcgaagaaattagcggcgtgaaactggaaagcattggcatttatcagattctgagcatttatagcaccgtggcgagcagcctggcgctggcgattatggtggcgggcctgagcctgtggatgtgcagcaacggcagcctgcagtgccgcatttgcatt

SEQ ID NO: 45 - DNA, 1707 bp, Influenza virus
atgaaagcgaaactgctggtgctgctgtatgcgtttgtggcgaccgatgcggataccatttgcattggctatcatgcgaacaacagcaccgataccgtggataccatttttgaaaaaaacgtggcggtgacccatagcgtgaacctgctggaagatcgccataacggcaaactgtgcaaactgaaaggcattgcgccgctgcagctgggcaaatgcaacattaccggctggctgctgggcaacccggaatgcgatagcctgctgccggcgcgcagctggagctatattgtggaaaccccgaacagcgaaaacggcgcgtgctatccgggcgattttattgattatgaagaactgcgcgaacagctgagcagcgtgagcagcctggaacgctttgaaatttttccgaaagaaagcagctggccgaaccatacctttaacggcgtgaccgtgagctgcagccatcgcggcaaaagcagcttttatcgcaacctgctgtggctgaccaaaaaaggcgatagctatccgaaactgaccaacagctatgtgaacaacaaaggcaaagaagtgctggtgctgtggggcgtgcatcatccgagcagcagcgatgaacagcagagcctgtatagcaacggcaacgcgtatgtgagcgtggcgagcagcaactataaccgccgctttaccccggaaattgcggcgcgcccgaaagtgaaagatcagcatggccgcatgaactattattggaccctgctggaaccgggcgataccattatttttgaagcgaccggcaacctgattgcgccgtggtatgcgtttgcgctgagccgcggctttgaaagcggcattattaccagcaacgcgagcatgcatgaatgcaacaccaaatgccagaccccgcagggcagcattaacagcaacctgccgtttcagaacattcatccggtgaccattggcgaatgcccgaaatatgtgcgcagcaccaaactgcgcatggtgaccggcctgcgcaacattccgagcattcagtatcgccgcaaaaaacgcggcctgtttggcgcgattgcgggctttattgaaggcggctggaccggcatgattgatggctggtatggctatcatcatcagaacgaacagggcagcggctatgcggcggatcagaaaagcacccagaacgcgattaacggcattaccaacaaagtgaacagcgtgattgaaaaaatgaacacccagtttaccgcggtgggcaaagaatttaacaacctggaaaaacgcatggaaaacctgaacaaaaaagtggatgatggctttctggatatttggacctataacgcggaactgctggtgctgctggaaaacgaacgcaccctggattttcatgatctgaacgtgaaaaacctgtatgaaaaagtgaaaagccagctgaaaaacaacgcgaaagaaattggcaacggctgctttgaattttatcataaatgcgataacgaatgcatggaaagcgtgcgcaacggcacctatgattatccgaaatatagcgaagaaagcaaactgaaccgcgaaaaaattgatggcgtgaaactggaaagcatgggcgtgtatcagattctggcgatttatagcaccgtggcgagcagcctggtgctgctggtgagcctgggcgcgattagcttttggatgtgcagcaacggcagcctgcagtgccgcatttgcatt